(19) United States
(12) Patent Application Publication
(10) Pub. No.: US 2018/0170983 A1
(43) Pub. Date: Jun. 21, 2018

(54) New Use of Cell-Permeable Peptide Inhibitors of the JNK Signal Transduction Pathway for the Treatment of Mild Cognitive Impairment

(71) Applicant: Xigen Inflammation Ltd., Limassol (CY)

(72) Inventors: Jean-Marc Combette, Saint Cergues (FR); Catherine Deloche, Genf (CH)

(21) Appl. No.: 15/737,480

(22) PCT Filed: Jun. 24, 2016
(86) PCT No.: PCT/EP2016/064765

§ 371 Date: Dec. 18, 2017

(30) Foreign Application Priority Data
Jun. 26, 2015 (EP) PCT/EP2015/001294
Oct. 8, 2015 (EP) PCT/EP2015/001974

(51) Int. Cl. C07K 14/47; A61K 38/17; A61K 45/06; C07K 16/18; A61P 25/28

(52) U.S. Cl. CPC C07K 14/4703; A61K 38/1709; C07K 2319/10; C07K 16/18; A61P 25/28; A61K 45/06

(57) ABSTRACT

The present invention refers to the use of protein kinase inhibitors and more specifically to the use of inhibitors of the protein kinase c-Jun N-terminal kinase, JNK inhibitor sequences, chimeric peptides, or of nucleic acids encoding same as well as pharmaceutical compositions containing same, for the prevention and/or treatment of Mild Cognitive Impairment, in particular of Mild Cognitive Impairment due to Alzheimer's Disease.

The present invention refers to the use of protein kinase inhibitors and more specifically to the use of inhibitors of the protein kinase c-Jun N-terminal kinase (JNK), JNK inhibitor sequences, chimeric peptides, or of nucleic acids encoding same as well as pharmaceutical compositions containing same, for the prevention and/or treatment of Mild Cognitive Impairment.

The c-Jun N-terminal kinase is a member of the stress-activated group of mitogen-activated protein (MAP) kinases. These kinases have been implicated in the control of cell growth and differentiation, and, more generally, in the response of cells to environmental stimuli. The JNK signal transduction pathway is activated in response to environmental stress and by the engagement of several classes of cell surface receptors. These receptors can include cytokine receptors, serpentine receptors and receptor tyrosine kinases. In mammalian cells, JNK has been implicated in biological processes such as oncogenic transformation and mediating adaptive responses to environmental stress. JNK has also been associated with modulating immune responses, including maturation and differentiation of immune cells, as well as effecting programmed cell death in cells identified for destruction by the immune system. This unique property makes JNK signaling a promising target for developing pharmacological intervention. Among several neurological disorders, JNK signaling is particularly implicated in ischemic stroke and Parkinson's disease, but also in other diseases as mentioned further below. It was also shown, that c-Jun N-terminal Kinase (JNK) is involved in neuropathic pain produced by spinal nerve ligation (SNL), wherein SNL induced a slow and persistent activation of JNK, in particular JNK1, wheras p38 mitogen-activated protein kinase activation was found in spinal microglia after SNL, which had fallen to near basal level by 21 days (Zhuang et al., The Journal of Neuroscience, Mar. 29, 2006, 26(13):3551-3560).

Inhibition or interruption of JNK signaling pathway, particularly the provision of inhibitors of the JNK signaling pathway, thus appears to be a promising approach in combating the above mentioned neurological disorders. However, there are only a few inhibitors of the JNK signaling pathway known so far.

Inhibitors of the JNK signaling pathway as already known in the prior art, particularly include e.g. upstream kinase inhibitors (for example, CEP-1347), small chemical inhibitors of JNK (SP600125 and AS601245), which directly affect kinase activity e.g. by competing with the ATP-binding site of the protein kinase, and peptide inhibitors of the interaction between JNK and its substrates (D-JNKI and 1-JIP) (see e.g. Kuan et al., Current Drug Targets—CNS & Neurological Disorders, February 2005, vol. 4, no. 1, pp. 63-67(5)).

The upstream kinase inhibitor CEP-1347 (KT7515) is a semisynthetic inhibitor of the mixed lineage kinase family. CEP-1347 (KT7515) promotes neuronal survival at dosages that inhibit activation of the c-Jun N-terminal kinases (JNKs) in primary embryonic cultures and differentiated PC12 cells after trophic withdrawal and in mice treated with 1-methyl-4-phenyl tetrahydropyridine. Further, CEP-1347 (KT7515) can promote long term-survival of cultured chick embryonic dorsal root ganglion, sympathetic, ciliary and motor neurons (see e.g. Borasio et al., Neuroreport. 9(7): 1435-1439, May 11 1998.).

The small chemical JNK inhibitor SP600125 was found to reduce the levels of c-Jun phosphorylation, to protect dopaminergic neurons from apoptosis, and to partly restore the level of dopamine in MPTP-induced PD in C57BL/6N mice (Wang et al., Neurosci Res. 2004 February; 48(2); 195-202). These results furthermore indicate that JNK pathway is the major mediator of the neurotoxic effects of MPTP in vivo and inhibiting JNK activity may represent a new and effective strategy to treat PD.

A further example of small chemical inhibitors is the aforementioned JNK-Inhibitor AS601245. AS601245 inhibits the JNK signalling pathway and promotes cell survival after cerebral ischemia. In vivo, AS601245 provided significant protection against the delayed loss of hippocampal CA1 neurons in a gerbil model of transient global ischemia. This effect is mediated by JNK inhibition and therefore by c-Jun expression and phosphorylation (see e.g. Carboni et al., J Pharmacol Exp Ther. 2004 July; 310(1):25-32. Epub 2004 February 26^{th}).

A third class of inhibitors of the JNK signaling pathway represent peptide inhibitors of the interaction between JNK and its substrates, as mentioned above. As a starting point for construction of such JNK inhibitor peptides a sequence alignment of naturally occurring JNK proteins may be used. Typically, these proteins comprise JNK binding domains (JBDs) and occur in various insulin binding (IB) proteins, such as IB1 or IB2. The results of such an exemplary sequence alignment is e.g. a sequence alignment between the JNK binding domains of IB1 [SEQ ID NO: 13], IB2 [SEQ ID NO: 14], c-Jun [SEQ ID NO: 15] and ATF2 [SEQ ID NO: 16] (see e.g. FIGS. 1A-1C). Such an alignment reveals a partially conserved 8 amino acid sequence (see e.g. FIG. 1A). A comparison of the JBDs of IB1 and IB2 further reveals two blocks of seven and three amino acids that are highly conserved between the two sequences.

Sequences constructed on basis of such an alignment are e.g. disclosed in WO 01/27268, in WO 2007/031280 or in WO 2009/144037. WO 2007/031280, WO 01/27268 and WO 2009/144037 disclose small cell permeable fusion peptides, comprising a so-called TAT cell permeation sequence derived from the basic trafficking sequence of the HIV-TAT protein and a minimum 20 amino acid inhibitory sequence of IB1. Both components are covalently linked to each other. Exemplary (and at present the only) inhibitors of the MAPK-JNK signaling pathway disclosed in WO 2007/031280, WO 01/27268 and WO 2009/144037, are e.g. L-JNKI1 (JNK-inhibitor peptide composed of L amino acids) or, in particular, the protease resistant D-JNKI1 peptides (JNK-inhibitor peptide composed of non-native D amino acids). These JNK-inhibitor (JNKI) peptides are specific for JNK (JNK1, JNK2 and JNK3). In contrast to those small compound inhibitors as discussed above, the inhibitor sequences in WO 2007/031280, WO 01/27268 and in WO 2009/144037, e.g. JNKI1, rather inhibit the interaction between JNK and its substrate. By its trafficking sequence derived from TAT, the fusion peptide is efficiently transported into cells. Due to the novel properties obtained by the trafficking component the fusion peptides are actively transported into cells, where they remain effective until proteolytic degradation.

WO 2009/144037 discloses in particular the use of such novel JNK inhibitor peptides in the treatment of Alzheimer's Disease. However, in contrast to Alzheimer's Disease (AD), Mild Cognitive Impairment (MCI) is diagnosed in patients without dementia. The absence of dementia is one of several important criteria to distinguish between AD and MCI.

Mild Cognitive Impairment is a syndrome defined as a subjective and objective decline in cognition and function greater than expected for an individual's age and education level that does not meet criteria for a diagnosis of dementia. However, elderly patients diagnosed with MCI constitute a high-risk population for developing dementia, in particular Alzheimer's disease (AD) but also other kinds of dementia, such as vascular dementia, frontotemporal dementia (FTD) and dementia with Lewy bodies (DLB). MCI is often referred to as an objective cognitive complaint for age, in a person with essentially normal functional activities, who does not have dementia. It affects 19% of people aged 65 and over. Around 46% of people with MCI develop dementia within 3 years compared with 3% of the population of the same age.

Despite the high incidence of Mild Cognitive Impairment, in particular among elderly patients, currently no medication is approved for treatment of Mild Cognitive Impairment. Since patients diagnosed with MCI are at a higher risk of developing dementia, such as Alzheimer's Disease and other dementia, many of the drugs that are approved for treating Alzheimer's Disease have been evaluated in several clinical trials as potential therapeutics for MCI. However, the drugs approved to treat Alzheimer's Disease have not shown any lasting benefit in MCI or in delaying or preventing progression of MCI to dementia.

Namely cholinesterase inhibitors, such as donepezil, galantamine, and rivastigmine, are approved for the treatment of Alzheimer's Disease. However, in clinical trials for MCI, none of the cholinesterase inhibitors showed promising results (C. Cooper et al., 2013, Treatment for Mild Cognitive Impairment: systematic review, The British Journal of Psychiatry 203: 255-264). In view thereof, it is recommended that cholinesterase inhibitors should not be prescribed clinically for MCI (National Institute for Health and Care Excellence. Dementia: supporting people with dementia and their careers in health and social care. Clinical Guideline 42. NICE, 2006; C. Cooper et al., 2013, Treatment for Mild Cognitive Impairment: systematic review, The British Journal of Psychiatry 203: 255-264).

In addition to cholinesterase inhibitors also other compounds, such as nicotine, gingko biloba, B vitamins, vitamin E and omega-3 polyunsaturated fatty acids, were evaluated as potential therapeutics for MCI, but the outcomes of those studies were either not promising or controversial.

In view of thereof it is an object of the present invention to identify a compound for preventing and/or treating Mild Cognitive Impairment (MCI), in particular MCI due to Alzheimer's Disease (AD).

This object is solved by the subject-matter of the appended claims, in particular by the use of a JNK inhibitor sequence, preferably as defined herein, typically comprising less than 150 amino acids in length for the preparation of a pharmaceutical composition for treating and/or preventing Mild Cognitive Impairment, in particular Mild Cognitive Impairment due to Alzheimer's Disease. In other words, this object is in particular solved by a JNK inhibitor sequence, preferably as defined herein, typically comprising less than 150 amino acids in length, for use in treating and/or preventing Mild Cognitive Impairment, in particular Mild Cognitive Impairment due to Alzheimer's Disease.

Although such a JNK inhibitor peptide was previously known as potential treatment of Alzheimer's Disease, its application in preventing and/or treating MCI is surprising. Of note, despite the many approved medications for AD, none of them showed promising results in clinical trials evaluating those drugs as therapeutics for MCI. This may be due to differences in the underlying neurobiological processes: certain pathways involved in Alzheimer's Disease may not (yet) be involved in Mild Cognitive Impairment. Drugs targeting such pathways may be well effective in Alzheimer's Disease, but of no use in preventing or treating MCI.

For example, E. J. Mufson et al., 2012, J Neuropathol Exp Neurol 71(11): 1018-1029 studied in subjects with no cognitive impairment (“NCI”), Mild Cognitive Impairment (MCI) and Alzheimer's Disease (AD) the involvement of ProNGF signaling pathways and their downstream protein kinase signaling pathways involved in pro-cell survival and pro-cell death actions, including Erk and protein kinase B/Akt, which activate intracellular events responsible for neuronal survival and neurite differentiation, as well as the c-jun kinase (JNK)-mediated proapoptotic pathway.

C-Jun N-terminal kinases (JNKs) are serine-threonine protein kinases, coded by three genes JNK1, JNK2, and JNK3, expressed as ten different isoforms by mRNA alternative splicing, each isoforms being expressed as a short form (46 kDa) and a long form (54 kDa) (Davis, 2000, Cell 103: 239-52). While JNK1 and JNK2 are ubiquitous, JNK3 is mainly expressed in the brain (Kyriakis and Avruch, 2001, Physiol Rev 81: 807-69). JNKs are activated by phosphorylation (pJNK) through MAPKinase activation by extracellular stimuli, such as ultraviolet stress, cytokines and Aβ peptides and they have multiple functions including gene expression regulation, cell proliferation and apoptosis (Dhanasekaran and Reddy, 2008, Oncogene 27: 6245-51).

Interestingly, E. J. Mufson et al., 2012, J Neuropathol Exp Neurol 71(11): 1018-1029 report no differences in JNK expression in subjects with no cognitive impairment (“NCI”), Mild Cognitive Impairment (MCI) and Alzheimer's Disease (AD). However, phospho-JNK and the phospho-JNK-to-JNK ratio (indicating the level of activated JNK) was significantly increased in the AD group as compared to the NCI and MCI groups. Moreover, the higher phospho-JNK levels in the AD group correlated with lower cognitive test scores, including episodic memory. In view thereof, JNK appears to be an attractive therapeutic target for Alzheimer's Disease. However, in the Mild-Cognitive-Impairment group no differences in JNK, phospho-JNK or in the phospho-JNK-to-JNK ratio were identified in comparison to the “No-cognitive-impairment” group. The more surprising was the finding of the present inventors that a JNK inhibitor is indeed useful as a therapeutic for MCI.

As described above, Mild Cognitive Impairment is typically distinct from Alzheimer's Disease. Accordingly, MCI is a disease on its own classified by ICD-10 in F06.7, whereas Alzheimer's Disease (AD) is classified by ICD-10 in G30. In other words, ICD-10 classifies MCI in an entirely different chapter (Chapter 5—Mental, Behavioral and Neurodevelopmental Disorders) than AD (Chapter 6—Diseases of the Nervous System).

In ICD-10 (F06.7), MCI is described as a disorder characterized by impairment of memory, learning difficulties, and reduced ability to concentrate on a task for more than brief periods. There is often a marked feeling of mental fatigue when mental tasks are attempted, and new learning is found to be subjectively difficult even when objectively successful. None of these symptoms is so severe that a diagnosis of either dementia (F00-F03) or delirium (F05.-) can be made. The disorder may precede, accompany, or follow a wide variety of infections and physical disorders, both cerebral and systemic, but direct evidence of cerebral involvement is not necessarily present. It can be differentiated from postencephalitic syndrome (F07.1) and postconcussional syndrome (F07.2) by its different etiology, more restricted range of generally milder symptoms, and usually shorter duration.

Mild Cognitive Impairment (MCI), in particular MCI due to Alzheimer's Disease, causes a slight but noticeable and measurable decline in cognitive abilities, including memory and thinking skills. MCI involves the onset and evolution of cognitive impairments whatever type beyond those expected based on the age and education of the individual, but which are not significant enough to interfere with their daily activities. The diagnosis of MCI is described for example by Albert M S, DeKosky S T, Dickson D, Dubois B, Feldman H H, Fox N C, Gamst A, Holtzman D M, Jagust W J, Petersen R C, Snyder P J, Carrillo M C, Thies B, Phelps C H (2011) The diagnosis of Mild Cognitive Impairment due to Alzheimer's disease: recommendations from the National Institute on Aging-Alzheimer's Association workgroups on diagnostic guidelines for Alzheimer's disease; Alzheimers Dement.; 7(3):270-9. MCI may be at the onset of whatever type of dementia or represents an ephemeric form of cognitive impairment which may disappear over time without resulting in a clinical manifestation of dementia. A person with MCI is at an increased risk of developing Alzheimer's or another dementia, in particular at an increased risk of developing Alzheimer's Disease, without however necessarily developing dementia, in particular Alzheimer's Disease. No medications are currently approved by the U.S. Food and Drug Administration (FDA) to treat Mild Cognitive Impairment. Drugs approved to treat symptoms of Alzheimer's Disease have not shown any lasting benefit in delaying or preventing progression of MCI to dementia.

Depending on whether single or multiple cognitive domains are affected, and whether there is a predominant memory complaint, different subtypes of MCI can be distinguished, namely
(i) amnestic MCI (a-MCI) if patients exhibit performance deficits on neuropsychological tests of episodic memory, or
(ii) non-amnestic MCI (na-MCI) if patients exhibit performance deficits on neuropsychological tests of non-memory domains of cognition.

Impairment could be limited to one cognitive domain (MCI single domain) or to multiple domains (MCI multiple domains). Therefore, patients could be classified as one of four possible clinical subtypes: 1) a-MCI single domain, 2) a-MCI multiple domain, 3) na-MCI single domain or 4) na-MCI multiple domain. The combination of clinical subtype and the presumed etiology (degenerative, vascular, psychiatric, trauma) could then be used to predict the type of dementia that the patient with MCI would most likely develop (AD, vascular dementia, frontotemporal dementia (FTD), dementia with Lewy bodies (DLB), etc.).

Preferably, the JNK inhibitor sequence as described herein is used for (the preparation of a medicament for) treating and/or preventing amnestic Mild Cognitive Impairment (a-MCI) or non-amnestic Mild Cognitive Impairment (na-MCI), preferably amnestic Mild Cognitive Impairment (a-MCI), more preferably Mild Cognitive Impairment due to Alzheimer's Disease (MCI due to AD).

MCI due to AD is a subtype of amnestic MCI (a-MCI). Biomarkers of AD, such as biomarkers of amyloid beta (Aβ) deposition and biomarkers of neuronal injury are recommended for diagnosis of MCI due to AD. Valid indicators of Aβ deposition include cerebrospinal fluid (CSF) concentrations of Aβ42 (decreased CSF Aβ42 levels) and positron emission tomography (PET) amyloid imaging. Valid indicators of neuronal injury include CSF concentrations of tau/phosphorylated tau (increased CSF tau/ptau levels), hippocampal volume or medial temporal atrophy or rate of brain atrophy on measured using structural MRI, and decreased glucose metabolism in temporoparietal regions on fluorodeoxyglucose (FDG) PET imaging.

Alzheimer's disease (AD) is a devastating neurodegenerative disorder that leads to progressive cognitive decline with memory loss and dementia. Neuropathological lesions are characterized by extracellular deposition of senile plaques, formed by β-amyloid (Aβ) peptide, and intracellular neurofibrillary tangles (NFTs), composed of hyperphosphorylated tau proteins (Duyckaerts et al., 2009, Acta Neuropathol 118: 5-36). According to the amyloid cascade hypothesis, neurodegeneratlon in AD could be linked to an abnormal amyloid precursor protein (APP) processing through the activity of the beta-site APP cleaving enzyme 1 (BACE1) and presenilin 1, leading to the production of toxic Aβ oligomers that accumulate in fibrillar Aβ peptides before forming Aβ plaques. AΔaccumulations can lead to synaptic dysfunction, altered kinase activities resulting in NFTs formation, neuronal loss and dementia (Hardy and Higgins, 1992, Science 256: 184-5). AD pathogenesis is thus believed to be triggered by the accumulation of Aβ, whereby Aβ self-aggregates into oligomers, which can be of various sizes, and forms diffuse and neuritic plaques in the parenchyma and blood vessels. Aβ oligomers and plaques are potent synaptotoxins, block proteasome function, inhibit mitochondrial activity, alter intracellular Ca²⁺ levels and stimulate inflammatory processes. Loss of the normal physiological functions of Aβ is also thought to contribute to neuronal dysfunction. Aβ interacts with the signalling pathways that regulate the phosphorylation of the microtubule-associated protein tau. Hyperphosphorylation of tau disrupts its normal function in regulating axonal transport and leads to the accumulation of neurofibrillary tangles (NFTs) and toxic species of soluble tau. Furthermore, degradation of hyperphosphorylated tau by the proteasome is inhibited by the actions of Aβ.

Typically, a JNK inhibitor sequence as defined herein may be derived from a human or rat IB1 sequence, preferably from an amino acid sequence as defined or encoded by any of sequences according to SEQ ID NO: 102 (depicts the IB1 cDNA sequence from rat and its predicted amino acid sequence), SEQ ID NO: 103 (depicts the IB1 protein sequence from rat encoded by the exon-intron boundary of the rIB1 gene—splice donor), SEQ ID NO: 104 (depicts the IB1 protein sequence from Homo sapiens), or SEQ ID NO: 105 (depicts the IB1 cDNA sequence from Homo sapiens), more preferably from an amino acid sequence as defined or encoded by any of sequences according to SEQ ID NO: 104 (depicts the IB1 protein sequence from Homo sapiens), or SEQ ID NO: 105 (depicts the IB1 cDNA sequence from Homo sapiens), or from any fragments or variants thereof. In other words, the JNK inhibitor sequence comprises a fragment, variant, or variant of such fragment of a human or rat IB1 sequence. Human or rat IB sequences are defined or encoded, respectively, by the sequences according to SEQ ID NO: 102, SEQ ID NO: 103, SEQ ID NO: 104 or SEQ ID NO: 105.

Preferably, such a JNK inhibitor sequence as used herein comprises a total length of less than 150 amino acid residues, preferably a range of 5 to 150 amino acid residues, more preferably 10 to 100 amino acid residues, even more preferably 10 to 75 amino acid residues and most preferably a range of 10 to 50 amino acid residues, e.g. 10 to 30, 10 to 20, or 10 to 15 amino acid residues.

More preferably, such a JNK inhibitor sequence and the above ranges may be selected from any of the above mentioned sequences, even more preferably from an amino acid sequence as defined according to SEQ ID NO: 104 or as encoded by SEQ ID NO: 105, even more preferably in the region between nucleotides 420 and 980 of SEQ ID NO: 105 or amino acids 105 and 291 of SEQ ID NO: 104, and most preferably in the region between nucleotides 561 and 647 of SEQ ID NO: 105 or amino acids 152 and 180 of SEQ ID NO: 104.

According to a particular embodiment, a JNK inhibitor sequence as used herein typically binds JNK and/or inhibits the activation of at least one JNK activated transcription factor, e.g. c-Jun or ATF2 (see e.g. SEQ ID NOs: 15 and 16, respectively) or Elk1.

Likewise, the JNK inhibitor sequence as used herein preferably comprises or consists of at least one amino acid sequence according to any one of SEQ ID NOs: 1 to 4, 13 to 20 and 33 to 100, or a fragment, derivative or variant thereof. More preferably, the JNK inhibitor sequence as used herein may contain 1, 2, 3, 4 or even more copies of an amino acid sequence according to SEQ ID NOs: 1 to 4, 13 to 20 and 33 to 100, or a variant, fragment or derivative thereof. If present in more than one copy, these amino acid sequences according to SEQ ID NOs: 1 to 4, 13 to 20 and 33 to 100, or variants, fragments, or derivatives thereof as used herein may be directly linked with each other without any linker sequence or via a linker sequence comprising 1 to 10, preferably 1 to 5 amino acids. Amino acids forming the linker sequence are preferably selected from glycine or proline as amino acid residues. More preferably, these amino acid sequences according to SEQ ID NOs: 1 to 4, 13 to 20 and 33 to 100, or fragments, variants or derivatives thereof, as used herein, may be separated by each other by a hinge of two, three or more proline residues.

The JNK inhibitor sequences as used herein may be composed of L-amino acids, D-amino acids, or a combination of both. Preferably, the JNK inhibitor sequences as used herein comprise at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the JNK inhibitor sequences as used herein in a blockwise, a non-blockwise or in an alternate manner.

According to one preferred embodiment the JNK inhibitor sequences as used herein may be exclusively composed of L-amino acids. The JNK inhibitor sequences as used herein may then comprise or consist of at least one “native JNK inhibitor sequence” according to SEQ ID NO: 1 or 3. In this context, the term “native” or “native JNK inhibitor sequence(s)” is referred to non-altered JNK inhibitor sequences according to any of SEQ ID NOs: 1 or 3, as used herein, entirely composed of L-amino acids.

Accordingly, the JNK inhibitor sequence as used herein may comprise or consist of at least one (native) amino acid sequence NH₂-Xₙ^{b}-Xₙ^{a}-RPTTLXLXXXXXXXQD-Xₙ^{b}-COOH (L-IB generic (s)) [SEQ ID NO: 3] and/or the JNK binding domain (JBDs) of IB1 XRPTTLXLXXXXXXXQDS/TX (L-IB (generic)) [SEQ ID NO: 19]. In this context, each X typically represents an amino acid residue, preferably selected from any (native) amino acid residue. Xₙ^{a} typically represents one amino acid residue, preferably selected from any amino acid residue except serine or threonine, wherein n (the number of repetitions of X) is 0 or 1. Furthermore, each Xₙ^{b} may be selected from any amino acid residue, wherein n (the number of repetitions of X) is 0-5, 5-10, 10-15, 15-20, 20-30 or more, provided that if n (the number of repetitions of X) is 0 for Xₙ^{a}, Xₙ^{b} does preferably not comprise a serine or threonine at its C-terminus, in order to avoid a serine or threonine at this position. Preferably, Xₙ^{b} represents a contiguous stretch of peptide residues derived from SEQ ID NO: 1 or 3. Xₙ^{a} and Xₙ^{b} may represent either D or L amino acids. Additionally, the JNK inhibitor sequence as used herein may comprise or consist of at least one (native) amino acid sequence selected from the group comprising the JNK binding domain of IB1 DTYRPKRPTTLNLFPQVPRSQDT (L-IB1) [SEQ ID NO: 17]. More preferably, the JNK inhibitor sequence as used herein further may comprise or consist of at least one (native) amino acid sequence NH₂-RPKRPTTLNLFPQVPRSQD-COOH (L-IB1(s)) [SEQ ID NO: 1]. Furthermore, the JNK inhibitor sequence as used herein may comprise or consist of at least one (native) amino acid sequence selected from the group comprising the JNK binding domain of IB1 L-IB1(s1) (NH₂-TLNLFPQVPRSQD-COOH, SEQ ID NO: 33); L-IB1(s2) (NH₂-TTLNLFPQVPRSQ-COOH, SEQ ID NO: 34); L-IB1(s3) (NH₂-PTTLNLFPQVPRS-COOH, SEQ ID NO: 35); L-IB1(s4) (NH₂-RPTTLNLFPQVPR-COOH, SEQ ID NO: 36); L-IB1(s5) (NH₂-KRPTTLNLFPQVP-COOH, SEQ ID NO: 37); L-IB1(s6) (NH₂-PKRPTTLNLFPQV-COOH, SEQ ID NO: 38); L-IB1(s7) (NH₂-RPKRPTTLNLFPQ-COOH, SEQ ID NO: 39); L-IB1(s8) (NH₂-LNLFPQVPRSQD-COOH, SEQ ID NO: 40); L-IB1(s9) (NH₂-TLNLFPQVPRSQ-COOH, SEQ ID NO: 41); L-IB1(s10) (NH₂-TTLNLFPQVPRS-COOH, SEQ ID NO: 42); L-IB1 (s1) (NH₂-PTTLNLFPQVPR-COOH, SEQ ID NO: 43); L-IB1(s12) (NH₂-RPTTLNLFPQVP-COOH, SEQ ID NO: 44); L-IB1(s13) (NH₂-KRPTTLNLFPQV-COOH, SEQ ID NO: 45); L-IB1(s14) (NH₂-PKRPTTLNLFPQ-COOH, SEQ ID NO: 46); L-IB1(s15) (NH₂-RPKRPTTLNLFP-COOH, SEQ ID NO: 47); L-IB1(s16) (NH₂-NLFPQVPRSQD-COOH, SEQ ID NO: 48); L-IB1 (s17) (NH₂-LNLFPQVPRSQ-COOH, SEQ ID NO: 49); L-IB1(s18) (NH₂-TLNLFPQVPRS-COOH, SEQ ID NO: 50); L-IB1(s19) (NH₂-TTLNLFPQVPR-COOH, SEQ ID NO: 51); L-IB1(s20) (NH₂-PTTLNLFPQVP-COOH, SEQ ID NO: 52); L-IB1(s21) (NH₂-RPTTLNLFPQV-COOH, SEQ ID NO: 53); L-IB1(s22) (NH₂-KRPTTLNLFPQ-COOH, SEQ ID NO: 54); L-IB1(s23) (NH₂-PKRPTTLNLFP-COOH, SEQ ID NO: 55); L-IB1(s24) (NH₂-RPKRPTTLNLF-COOH, SEQ ID NO: 56); L-IB1(s25) (NH₂-LFPQVPRSQD-COOH, SEQ ID NO: 57); L-IB1(s26) (NH₂-NLFPQVPRSQ-COOH, SEQ ID NO: 58); L-IB1(s27) (NH₂-LNLFPQVPRS-COOH, SEQ ID NO: 59); L-IB1(s28) (NH₂-TLNLFPQVPR-COOH, SEQ ID NO: 60); L-IB1(s29) (NH₂-TTLNLFPQVP-COOH, SEQ ID NO: 61); L-IB1(s30) (NH₂-PTTLNLFPQV-COOH, SEQ ID NO: 62); L-IB1(s31) (NH₂-RPTTLNLFPQ-COOH, SEQ ID NO: 63); L-IB1(s32) (NH₂-KRPTTLNLFP-COOH, SEQ ID NO: 64); L-IB1(s33) (NH₂-PKRPTTLNLF-COOH, SEQ ID NO: 65); and L-IB1(s34) (NH₂-RPKRPTTLNL-COOH, SEQ ID NO: 66).

Additionally, the JNK inhibitor sequence as used herein may comprise or consist of at least one (native) amino acid sequence selected from the group comprising the (long) JNK binding domain (JBDs) of IB1 PGTGCGDTYRPKRPTTLNLFPQVPRSQDT (IB1-long) [SEQ ID NO: 13], the (long) JNK binding domain of IB2 IPSPSVEEPHKHRPTTLRLTTLGAQDS (IB2-long) [SEQ ID NO: 14], the JNK binding domain of c-Jun GAYGYSNPKILKQSMTLNLADPVGNLKPH (c-Jun) [SEQ ID NO: 15], the JNK binding domain of ATF2 TNEDHLAVHKHIKHEMTLKFGPARNDSVIV (ATF2) [SEQ ID NO: 16] (see e.g. FIG. 1A-1C). In this context, an alignment revealed a partially conserved 8 amino acid sequence (see e.g. FIG. 1A) and a further comparison of the JBDs of IB1 and IB2 revealed two blocks of seven and three amino acids that are highly conserved between the two sequences.

According to another preferred embodiment the JNK inhibitor sequences as used herein may be composed in part or exclusively of D-amino acids as defined above. More preferably, these JNK inhibitor sequences composed of D-amino acids are non-native D retro-inverso sequences of the above (native) JNK inhibitor sequences. The term “retro-inverso sequences” refers to an isomer of a linear peptide sequence in which the direction of the sequence is reversed and the chirality of each amino acid residue is inverted (see e.g. Jameson et al., Nature, 368, 744-746 (1994); Brady et al., Nature, 368, 692-693 (1994)). The advantage of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence or peptide as used according to the present invention may be converted into an D retro-inverso sequence or peptide by synthesizing a reverse of the sequence or peptide for the corresponding native L-amino acid sequence or peptide.

The D retro-inverso sequences as used herein and as defined above have a variety of useful properties. For example, D retro-inverso sequences as used herein enter cells as efficiently as L-amino acid sequences as used herein, whereas the D retro-inverso sequences as used herein are more stable than the corresponding L-amino acid sequences.

Accordingly, the JNK inhibitor sequences as used herein may comprise or consist of at least one D retro-inverso sequence according to the amino acid sequence NH₂-Xₙ^{b}, DQXXXXXXXLXLTTPR-Xₙ^{a}-Xₙ^{b}-COOH (D-IB1 generic (s)) [SEQ ID NO: 4] and/or XS/TDQXXXXXXXLXLTTPRX (D-IB (generic)) [SEQ ID NO: 20]. As used in this context, X, Xₙ^{a} and Xₙ^{b} are as defined above (preferably, representing D amino acids), wherein Xₙ^{b} preferably represents a contiguous stretch of residues derived from SEQ ID NO: 2 or 4. Additionally, the JNK inhibitor sequences as used herein may comprise or consist of at least one D retro-inverso sequence according to the amino acid sequence comprising the JNK binding domain (JBDs) of IB1 TDQSRPVQPFLNLTTPRKPRYTD (D-IB1) [SEQ ID NO: 18]. More preferably, the JNK inhibitor sequences as used herein may comprise or consist of at least one D retro-inverso sequence according to the amino acid sequence NH₂-DQSRPVQPFLNLTTPRKPR-COOH (D-IB1 (s)) [SEQ ID NO: 2]. Furthermore, the JNK inhibitor sequences as used herein may comprise or consist of at least one D retro-inverso sequence according to the amino acid sequence comprising the JNK binding domain (JBDs) of IB1 D-IB1(s1) (NH₂-QPFLNLTTPRKPR-COOH, SEQ ID NO: 67); D-IB1(s2) (NH₂-VQPFLNLTTPRKP-COOH, SEQ ID NO: 68); D-IB1(s3) (NH₂-PVQPFLNLTTPRK-COOH, SEQ ID NO: 69); D-IB1 (s4) (NH₂-RPVQPFLNLTTPR-COOH, SEQ ID NO: 70); D-IB1 (s5) (NH₂-SRPVQPFLNLTTP-COOH, SEQ ID NO: 71); D-IB1(s6) (NH₂-QSRPVQPFLNLTT-COOH, SEQ ID NO: 72); D-IB1 (s7) (NH₂-DQSRPVQPFLNLT-COOH, SEQ ID NO: 73); D-IB1 (s8) (NH₂-PFLNLTTPRKPR-COOH, SEQ ID NO: 74); D-IB1(s9) (NH₂-QPFLNLTTPRKP-COOH, SEQ ID NO: 75); D-IB1(s10) (NH₂-VQPFLNLTTPRK-COOH, SEQ ID NO: 76); D-IB1(s11) (NH₂-PVQPFLNLTTPR-COOH, SEQ ID NO: 77); D-IB1(s12) (NH₂-RPVQPFLNLTTP-COOH, SEQ ID NO: 78); D-IB1 (s13) (NH₂-SRPVQPFLNLTT-COOH, SEQ ID NO: 79); D-IB1 (s14) (NH₂-QSRPVQPFLNLT-COOH, SEQ ID NO: 80); D-IB1(s15) (NH₂-DQSRPVQPFLNL-COOH, SEQ ID NO: 81); D-IB1(s16) (NH₂-FLNLTTPRKPR-COOH, SEQ ID NO: 82); D-IB1(s17) (NH₂-PFLNLTTPRKP-COOH, SEQ ID NO: 83); D-IB1(s18) (NH₂-QPFLNLTTPRK-COOH, SEQ ID NO: 84); D-IB1(s19) (NH₂-VQPFLNLTTPR-COOH, SEQ ID NO: 85); D-IB1(s20) (NH₂-PVQPFLNLTTP-COOH, SEQ ID NO: 86); D-IB1(s21) (NH₂-RPVQPFLNLTT-COOH, SEQ ID NO: 87); D-IB1(s22) (NH₂-SRPVQPFLNLT-COOH, SEQ ID NO: 88); D-IB1(s23) (NH₂-QSRPVQPFLNL-COOH, SEQ ID NO: 89); D-IB1(s24) (NH₂-DQSRPVQPFLN-COOH, SEQ ID NO: 90); D-IB1(s25) (NH₂-DQSRPVQPFL-COOH, SEQ ID NO: 91); D-IB1(s26) (NH₂-QSRPVQPFLN-COOH, SEQ ID NO: 92); D-IB1(s27) (NH₂-SRPVQPFLNL-COOH, SEQ ID NO: 93); D-IB1(s28) (NH₂-RPVQPFLNLT-COOH, SEQ ID NO: 94); D-IB1(s29) (NH₂-PVQPFLNLTT-COOH, SEQ ID NO: 95); D-IB1(s30) (NH₂-VQPFLNLTTP-COOH, SEQ ID NO: 96); D-IB1(s31) (NH₂-QPFLNLTTPR-COOH, SEQ ID NO: 97); D-IB1(s32) (NH₂-PFLNLTTPRK-COOH, SEQ ID NO: 98); D-IB1(s33) (NH₂-FLNLTTPRKP-COOH, SEQ ID NO: 99); and D-IB1(s34) (NH₂-LNLTTPRKPR-COOH, SEQ ID NO: 100).

The JNK inhibitor sequences as used herein and as disclosed above are presented in Table 1 (SEQ ID NO:s1-4, 13-20 and 33-100). The table presents the name of the JNK inhibitor sequences as used herein, as well as their sequence identifier number, their length, and amino acid sequence. Furthermore, Table 1 shows sequences as well as their generic formulas, e.g. for SEQ ID NO's: 1, 2, 5, 6, 9 and 11 and SEQ ID NO's: 3, 4, 7, 8, 10 and 12, respectively. Table 1 furthermore discloses the chimeric sequences SEQ ID NOs: 9-12 and 23-32 (see below), L-IB1 sequences SEQ ID NOs: 33 to 66 and D-IB1 sequences SEQ ID NOs: 67 to 100.

TABLE 1

| | SEQ | | |
|---|---|---|---|
| SEQUENCE/PEPTIDE | ID | | |
| NAME | NO | AA | SEQUENCE |
| L-IB1(s) | 1 | 19 | RPKRPTTLNLFPQVPRSQD |
| | | | (NH₂-RPKRPTTLNLFPQVPRSQD-COOH) |
| D-IB1(s) | 2 | 19 | DQSRPVQPFLNLTTPRKPR |
| | | | (NH₂-DQSRPVQPFLNLTTPRKPR-COOH) |
| L-IB (generic) (s) | 3 | 19 | NH₂-Xₙ^{b}-Xₙ^{a}-RPTTLXLXXXXXXXQD-Xₙ^{b}-COOH |
| D-IB (generic) (s) | 4 | 19 | NH₂-Xₙ^{b}-DQXXXXXXXLXLTTPR-Xₙ^{a}-Xₙ^{b}-COOH |
| L-TAT | 5 | 10 | GRKKRRQRRR |
| | | | (NH₂-GRKKRRQRRR-COOH) |
| D-TAT | 6 | 10 | RRRQRRKKRG |
| | | | (NH₂-RRRQRRKKRG-COOH) |
| L-generic-TAT (s) | 7 | 11 | NH₂-Xₙ^{b}-RKKRRQRRR-Xₙ^{b}-COOH |
| D-generic-TAT (s) | 8 | 11 | NH₂-Xₙ^{b}-RRRQRRKKR-Xₙ^{b}-COOH |
| L-TAT-IB1(s) | 9 | 31 | GRKKRRQRRRPPRPKRPTTLNLFPQVPRSQD |
| | | | (NH₂-GRKKRRQRRRPPRPKRPTTLNLFPQVPRSQD-COOH) |
| L-TAT-IB (generic) (s) | 10 | 29 | NH₂-Xₙ^{b}-RKKRRQRRR-Xₙ^{b}-Xₙ^{a}-RPTTLXLXXXXXXXQD-Xₙ^{b}-COOH |
| D-TAT-IB1(s) | 11 | 31 | DQSRPVQPFLNLTTPRKPRPPRRRQRRKKRG |
| | | | (NH₂-DQSRPVQPFLNLTTPRKPRPPRRRQRRKKRG-COOH) |
| D-TAT-IB (generic) (s) | 12 | 29 | NH₂-Xₙ^{b}-DQXXXXXXXLXLTTPR-Xₙ^{a}-Xₙ^{b}-RRRQRRKKR-Xₙ^{b}-COOH |
| IB1-long | 13 | 29 | PGTGCGDTYRPKRPTTLNLFPQVPRSQDT |
| | | | (NH₂-PGTGCGDTYRPKRPTTLNLFPQVPRSQDT-COOH) |
| IB2-long | 14 | 27 | IPSPSVEEPHKHRPTTLRLTTLGAQDS |
| | | | (NH₂-IPSPSVEEPHKHRPTTLRLTTLGAQDS-COOH) |
| c-Jun | 15 | 29 | GAYGYSNPKILKQSMTLNLADPVGNLKPH |
| | | | (NH₂-GAYGYSNPKILKQSMTLNLADPVGNLKPH-COOH) |
| ATF2 | 16 | 29 | TNEDHLAVHKHKHEMTLKFGPARNDSVIV |
| | | | (NH₂-TNEDHLAVHKHKHEMTLKFGPARNDSVIV-COOH) |
| L-IB1 | 17 | 23 | DTYRPKRPTTLNLFPQVPRSQDT |
| | | | (NH₂-DTYRPKRPTTLNLFPQVPRSQDT-COOH) |
| D-IB1 | 18 | 23 | TDQSRPVQPFLNLTTPRKPRYTD |
| | | | (NH₂-TDQSRPVQPFLNLTTPRKPRYTD-COOH) |
| L-IB (generic) | 19 | 19 | XRPTTLXLXXXXXXXQDS/TX |
| | | | (NH₂-XRPTTLXLXXXXXXXQDS/TX-COOH) |
| D-IB (generic) | 20 | 19 | XS/TDQXXXXXXXLXLTTPRX |
| | | | (NH₂-XS/TDQXXXXXXXLXLTTPRX-COOH) |
| L-generic-TAT | 21 | 17 | XXXXRKKRRQRRRXXXX |
| | | | (NH₂-XXXXRKKRRQRRRXXXX-COOH) |
| D-generic-TAT | 22 | 17 | XXXXRRRQRRKKRXXXX |
| | | | (NH₂-XXXXRRRQRRKKRXXXX-COOH) |
| L-TAT-IB1 | 23 | 35 | GRKKRRQRRRPPDTYRPKRPTTLNLFPQVPRSQDT |
| | | | (NH₂-GRKKRRQRRRPPDTYRPKRPTTLNLFPQVPRSQDT-COOH) |
| L-TAT-IB (generic) | 24 | 42 | XXXXXXXRKKRRQRRRXXXXXXXXRPTTLXLXXXXXXXQDS/TX |
| | | | (NH₂- |
| | | | XXXXXXXRKKRRQRRRXXXXXXXXRPTTLXLXXXXXXXQDS/TX- |
| | | | COOH) |
| D-TAT-IB1 | 25 | 35 | TDQSRPVQPFLNLTTPRKPRYTDPPRRRQRRKKRG |
| | | | (NH₂-TDQSRPVQPFLNLTTPRKPRYTDPPRRRQRRKKRG-COOH) |
| D-TAT-IB (generic) | 26 | 42 | XT/SDQXXXXXXXLXLTTPRXXXXXXXXRRRQRRKKRXXXXXXX |
| | | | (NH₂- |
| | | | XT/SDQXXXXXXXLXLTTPRXXXXXXXXRRRQRRKKRXXXXXXX- |
| | | | COOH) |
| L-TAT-IB1(s1) | 27 | 30 | RKKRRQRRRPPRPKRPTTLNLFPQVPRSQD |
| | | | (NH₂-RKKRRQRRRPPRPKRPTTLNLFPQVPRSQD-COOH) |
| L-TAT-IB1(s2) | 28 | 30 | GRKKRRQRRRXₙ^{c}RPKRPTTLNLFPQVPRSQD |
| | | | (NH₂-GRKKRRQRRRXₙ^{c}RPKRPTTLNLFPQVPRSQD-COOH) |
| L-TAT-IB1(s3) | 29 | 29 | RKKRRQRRRXₙ^{c}RPKRPTTLNLFPQVPRSQD |
| | | | (NH₂-RKKRRQRRRXₙ^{c}RPKRPTTLNLFPQVPRSQD-COOH) |
| D-TAT-IB1(s1) | 30 | 30 | DQSRPVQPFLNLTTPRKPRPPRRRQRRKKR |
| | | | (NH₂-DQSRPVQPFLNLTTPRKPRPPRRRQRRKKR-COOH) |
| D-TAT-IB1(s2) | 31 | 30 | DQSRPVQPFLNLTTPRKPRXₙ^{c}RRRQRRKKRG |
| | | | (NH₂-DQSRPVQPFLNLTTPRKPRVRRRQRRKKRG-COOH) |
| D-TAT-IB1(s3) | 32 | 29 | DQSRPVQPFLNLTTPRKPRXₙ^{c}RRRQRRKKR |
| | | | (NH₂-DQSRPVQPFLNLTTPRKPRXRRRQRRKKR-COOH) |
| L-IB1(s1) | 33 | 13 | TLNLFPQVPRSQD |
| | | | (NH₂-TLNLFPQVPRSQD-COOH) |
| L-IB1(s2) | 34 | 13 | TTLNLFPQVPRSQ |
| | | | (NH₂-TTLNLFPQVPRSQ-COOH) |
| L-IB1(s3) | 35 | 13 | PIILNLFPQVPRS |
| | | | (NH₂-PTTLNLFPQVPRS-COOH) |
| L-IB1(s4) | 36 | 13 | RPTTLNLFPQVPR |
| | | | (NH₂-RPTTLNLFPQVPR-COOH) |
| L-IB1(s5) | 37 | 13 | KRPTTLNLFPQVP |
| | | | (NH₂-KRPTTLNLFPQVP-COOH) |
| L-IB1(s6) | 38 | 13 | PKRPTTLNLFPQV |
| | | | (NH₂-PKRPTTLNLFPQV-COOH) |
| L-IB1(s7) | 39 | 13 | RPKRPTTLNLFPQ |
| | | | (NH₂-RPKRPTTLNLFPQ-COOH) |
| L-IB1(s8) | 40 | 12 | LNLFPQVPRSQD |
| | | | (NH₂-LNLFPQVPRSQD-COOH) |
| L-IB1(s9) | 41 | 12 | TLNLFPQVPRSQ |
| | | | (NH₂-TLNLFPQVPRSQ-COOH) |
| L-IB1(s10) | 42 | 12 | TTLNLFPQVPRS |
| | | | (NH₂-TTLNLFPQVPRS-COOH) |
| L-IB1(s11) | 43 | 12 | PTTLNLFPQVPR |
| | | | (NH₂-PTTLNLFPQVPR-COOH) |
| L-IB1(s12) | 44 | 12 | RPTTLNLFPQVP |
| | | | (NH₂-RPTTLNLFPQVP-COOH) |
| L-IB1(s13) | 45 | 12 | KRPTTLNLFPQV |
| | | | (NH₂-KRPTTLNLFPQV-COOH) |
| L-IB1(s14) | 46 | 12 | PKRPTTLNLFPQ |
| | | | (NH₂-13KRPTTLNLFPQ-COOH) |
| L-IB1(s15) | 47 | 12 | RPKRPTTLNLFP |
| | | | (NH₂-RPKRPTTLNLFP-COOH) |
| L-IB1(s16) | 48 | 11 | NLFPQVPRSQD |
| | | | (NH₂-NLFPQVPRSQD-COOH) |
| L-IB1(s17) | 49 | 11 | LNLFPQVPRSQ |
| | | | (NH₂-LNLFPQVPRSQ-COOH) |
| L-IB1(s18) | 50 | 11 | TLNLFPQVPRS |
| | | | (NH₂-TLNLFPQVPRS-COOH) |
| L-IB1(s19) | 51 | 11 | TTLNLFPQVPR |
| | | | (NH₂-TTLNLFPQVPR-COOH) |
| L-IB1(s20) | 52 | 11 | PTTLNLFPQVP |
| | | | (NH₂-PTTLNLFPQVP-COOH) |
| L-IB1(s21) | 53 | 11 | RPTTLNLFPQV |
| | | | (NH₂-RPTTLNLFPQV-COOH) |
| L-IB1(s22) | 54 | 11 | KRPTTLNLFPQ |
| | | | (NH₂-KRPIILNLFPQ-COOH) |
| L-IB1(s23) | 55 | 11 | PKRPTTLNLFP |
| | | | (NH₂-PKRPTTLNLFP-COOH) |
| L-IB1(s24) | 56 | 11 | RPKRPTTLNLF |
| | | | (NH₂-RPKRPTTLNLF-COOH) |
| L-IB1(s25) | 57 | 10 | LFPQVPRSQD |
| | | | (NH₂-LFPQVPRSQD-COOH) |
| L-IB1(s26) | 58 | 10 | NLFPQVPRSQ |
| | | | (NH₂-NLFPQVPRSQ-COOH) |
| L-IB1(s27) | 59 | 10 | LNLFPQVPRS |
| | | | (NH₂-LNLFPQVPRS-COOH) |
| L-IB1(s28) | 60 | 10 | TLNLFPQVPR |
| | | | (NH₂-TLNLFPQVPR-COOH) |
| L-IB1(s29) | 61 | 10 | TTLNLFPQVP |
| | | | (NH₂-TTLNLFPQVP-COOH) |
| L-IB1(s30) | 62 | 10 | PTTLNLFPQV |
| | | | (NH₂-PTTLNLFPQV-COOH) |
| L-IB1(s31) | 63 | 10 | RPTTLNLFPQ |
| | | | (NH₂-RPTTLNLFPQ-COOH) |
| L-IB1(s32) | 64 | 10 | KRPTTLNLFP |
| | | | (NH₂-KRPTTLNLFP-COOH) |
| L-IB1(s33) | 65 | 10 | PKRPTTLNLF |
| | | | (NH₂-PKRPTTLNLF-COOH) |
| L-IB1(s34) | 66 | 10 | RPKRPTTLNL |
| | | | (NH₂-RPKRPTTLNL-COOH) |
| D-IB1(s1) | 67 | 13 | QPFLNLTTPRKPR |
| | | | (NH₂-QPFLNLTTPRKPR-COOH) |
| D-IB1(s2) | 68 | 13 | VQPFLNLTTPRKP |
| | | | (NH₂-VQPFLNLTTPRKP-COOH) |
| D-IB1(s3) | 69 | 13 | PVQPFLNLTTPRK |
| | | | (NH₂-PVQPFLNLTTPRK-COOH) |
| D-IB1(s4) | 70 | 13 | RPVQPFLNLTTPR |
| | | | (NH₂-RPVQPFLNLTTPR-COOH) |
| D-IB1(s5) | 71 | 13 | SRPVQPFLNLTTP |
| | | | (NH₂-SRPVQPFLNLTTP-COOH) |
| D-IB1(s6) | 72 | 13 | QSRPVQPFLNLTT |
| | | | (NH₂-QSRPVQPFLNLTT-COOH) |
| D-IB1(s7) | 73 | 13 | DQSRPVQPFLNLT |
| | | | (NH₂-DQSRPVQPFLNLT-COOH) |
| D-IB1(s8) | 74 | 12 | PFLNLTTPRKPR |
| | | | (NH₂-PFLNLTTPRKPR-COOH) |
| D-IB1(s9) | 75 | 12 | QPFLNLTTPRKP |
| | | | (NH₂-QPFLNLTTPRKP-COOH) |
| D-IB1(s10) | 76 | 12 | VQPFLNLTTPRK |
| | | | (NH₂-VQPFLNLTTPRK-COOH) |
| D-IB1(s11) | 77 | 12 | PVQPFLNLTTPR |
| | | | (NH₂-PVQPFLNLTTPR-COOH) |
| D-IB1(s12) | 78 | 12 | RPVQPFLNLTTP |
| | | | (NH₂-RPVQPFLNLTTP-COOH) |
| D-IB1(s13) | 79 | 12 | SRPVQPFLNLTT |
| | | | (NH₂-SRPVQPFLNLTT-COOH) |
| D-IB1(s14) | 80 | 12 | QSRPVQPFLNLT |
| | | | (NH₂-QSRPVQPFLNLT-COOH) |
| D-IB1(s15) | 81 | 12 | DQSRPVQPFLNL |
| | | | (NH₂-DQSRPVQPFLNL-COOH) |
| D-IB1(s16) | 82 | 11 | FLNLTTPRKPR |
| | | | (NH₂-FLNLTTPRKPR-COOH) |
| D-IB1(s17) | 83 | 11 | PFLNLTTPRKP |
| | | | (NH₂-PFLNLTTPRKP-COOH) |
| D-IB1(s18) | 84 | 11 | QPFLNLTTPRK |
| | | | (NH₂-QPFLNLTTPRK-COOH) |
| D-IB1(s19) | 85 | 11 | VQPFLNLTTPR |
| | | | (NH₂-VQPFLNLTTPR-COOH) |
| D-IB1(s20) | 86 | 11 | PVQPFLNLTTP |
| | | | (NH₂-PVQPFLNLTTP-COOH) |
| D-IB1(s21) | 87 | 11 | RPVQPFLNLTT |
| | | | (NH₂-RPVQPFLNLTT-COOH) |
| D-IB1(s22) | 88 | 11 | SRPVQPFLNLT |
| | | | (NH₂-SRPVQPFLNLT-COOH) |
| D-IB1(s23) | 89 | 11 | QSRPVQPFLNL |
| | | | (NH₂-QSRPVQPFLNL-COOH) |
| D-IB1(s24) | 90 | 11 | DQSRPVQPFLN |
| | | | (NH₂-DQSRPVQPFLN-COOH) |
| D-IB1(s25) | 91 | 10 | DQSRPVQPFL |
| | | | (NH₂-DQSRPVQPFL-COOH) |
| D-IB1(s26) | 92 | 10 | QSRPVQPFLN |
| | | | (NH₂-QSRPVQPFLN-COOH) |
| D-IB1(s27) | 93 | 10 | SRPVQPFLNL |
| | | | (NH₂-SRPVQPFLNL-COOH) |
| D-IB1(s28) | 94 | 10 | RPVQPFLNLT |
| | | | (NH₂-RPVQPFLNLT-COOH) |
| D-IB1(s29) | 95 | 10 | PVQPFLNLTT |
| | | | (NH₂-PVQPFLNLTT-COOH) |
| D-IB1(s30) | 96 | 10 | VQPFLNLTTP |
| | | | (NH₂-VQPFLNLTTP-COOH) |
| D-IB1(s31) | 97 | 10 | QPFLNLTTPR |
| | | | (NH₂-QPFLNLTTPR-COOH) |
| D-IB1(s32) | 98 | 10 | PFLNLTTPRK |
| | | | (NH₂-PFLNLTTPRK-COOH) |
| D-IB1(s33) | 99 | 10 | FLNLTTPRKP |
| | | | (NH₂-FLNLTTPRKP-COOH) |
| D-IB1(s34) | 100 | 10 | LNLTTPRKPR |
| | | | (NH₂-LNLTTPRKPR-COOH) |

According to another preferred embodiment, the JNK inhibitor sequence as used herein comprises or consists of at least one variant, fragment and/or derivative of the above defined native or non-native amino acid sequences according to SEQ ID NOs: 1-4, 13-20 and 33-100. Preferably, these variants, fragments and/or derivatives retain biological activity of the above disclosed native or non-native JNK inhibitor sequences as used herein, particularly of native or non-native amino acid sequences according to SEQ ID NOs: 1-4, 13-20 and 33-100, i.e. binding JNK and/or inhibiting the activation of at least one JNK activated transcription factor, e.g. c-Jun, ATF2 or Elk1. Functionality may be tested by various tests, e.g. binding tests of the peptide to its target molecule or by biophysical methods, e.g. spectroscopy, computer modeling, structural analysis, etc. Particularly, an JNK inhibitor sequence or variants, fragments and/or derivatives thereof as defined above may be analyzed by hydrophilicity analysis (see e.g. Hopp and Woods, 1981. Proc Natl Acad Sci USA 78: 3824-3828) that can be utilized to identify the hydrophobic and hydrophilic regions of the peptides, thus aiding in the design of substrates for experimental manipulation, such as in binding experiments, or for antibody synthesis. Secondary structural analysis may also be performed to identify regions of an JNK inhibitor sequence or of variants, fragments and/or derivatives thereof as used herein that assume specific structural motifs (see e.g. Chou and Fasman, 1974, Biochem 13: 222-223). Manipulation, translation, secondary structure prediction, hydrophilicity and hydrophobicity profiles, open reading frame prediction and plotting, and determination of sequence homologies can be accomplished using computer software programs available in the art. Other methods of structural analysis include, e.g. X-ray crystallography (see e.g. Engstrom, 1974. Biochem Exp Biol 11: 7-13), mass spectroscopy and gas chromatography (see e.g. METHODS IN PROTEIN SCIENCE, 1997, J. Wiley and Sons, New York, N.Y.) and computer modeling (see e.g. Fletterick and Zoller, eds., 1986. Computer Graphics and Molecular Modeling, In: CURRENT COMMUNICATIONS IN MOLECULAR BIOLOGY, Cold Spring Harbor Laboratory Press, Cold Spring Harbor, N.Y.) may also be employed.

Accordingly, the JNK inhibitor sequence as used herein may comprise or consist of at least one variant of (native or non-native) amino acid sequences according to SEQ ID NOs: 1-4, 13-20 and 33-100. In the context of the present invention, a “variant of a (native or non-native) amino acid sequence according to SEQ ID NOs: 1-4, 13-20 and 33-100” is preferably a sequence derived from any of the sequences according to SEQ ID NOs: 1-4, 13-20 and 33-100, wherein the variant comprises amino acid alterations of the amino acid sequences according to SEQ ID NOs: 1-4, 13-20 and 33-100. Such alterations typically comprise 1 to 20, preferably 1 to 10 and more preferably 1 to 5 substitutions, additions and/or deletions of amino acids according to SEQ ID NOs: 1-4, 13-20 and 33-100, wherein the variant exhibits a sequence identity with any of the sequences according to SEQ ID NOs: 1-4, 13-20 and 33-100 of at least about 30%, 50%, 70%, 80%, 90%, 95%, 98% or even 99%.

If variants of (native or non-native) amino acid sequences according to SEQ ID NOs: 1-4, 13-20 and 33-100 as defined above and used herein are obtained by substitution of specific amino acids, such substitutions preferably comprise conservative amino acid substitutions. Conservative amino acid substitutions may include synonymous amino acid residues within a group which have sufficiently similar physicochemical properties, so that a substitution between members of the group will preserve the biological activity of the molecule (see e.g. Grantham, R. (1974), Science 185, 862-864). It is evident to the skilled person that amino acids may also be inserted and/or deleted in the above-defined sequences without altering their function, particularly if the insertions and/or deletions only involve a few amino acids, e.g. less than twenty, and preferably less than ten, and do not remove or displace amino acids which are critical to functional activity. Moreover, substitutions shall be avoided in variants as used herein, which lead to additional threonines at amino acid positions which are accessible for a phosphorylase, preferably a kinase, in order to avoid inactivation of the JNK-inhibitor sequence as used herein or of the chimeric peptide as used herein in vivo or in vitro.

Preferably, synonymous amino acid residues, which are classified into the same groups and are typically exchangeable by conservative amino acid substitutions, are defined in Table 2.

TABLE 2
Preferred Groups of Synonymous Amino Acid Residues

| Amino Acid | Synonymous Residue |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, (Thr), Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, (Thr), Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, (Thr), Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

A specific form of a variant of SEQ ID NOs: 1-4, 13-20 and 33-100 as used herein is a fragment of the (native or non-native) amino acid sequences according to SEQ ID NOs: 1, 1-4, 13-20 and 33-100″ as used herein, which is typically altered by at least one deletion as compared to SEQ ID NOs 1-4, 13-20 and 33-100. Preferably, a fragment comprises at least 4 contiguous amino acids of any of SEQ ID NOs: 1-4, 13-20 and 33-100, a length typically sufficient to allow for specific recognition of an epitope from any of these sequences. Even more preferably, the fragment comprises 4 to 18, 4 to 15, or most preferably 4 to 10 contiguous amino acids of any of SEQ ID NOs: 1-4, 13-20 and 33-100, wherein the lower limit of the range may be 4, or 5, 6, 7, 8, 9, or 10. Deleted amino acids may occur at any position of SEQ ID NOs: 1-4, 13-20 and 33-100, preferably N- or C-terminally.

Furthermore, a fragment of the (native or non-native) amino acid sequences according to SEQ ID NOs: 1-4, 13-20 and 33-100, as described above, may be defined as a sequence sharing a sequence identity with any of the sequences according to SEQ ID NOs: 1-4, 13-20 and 33-100 as used herein of at least about 30%, 50%, 70%, 80%, 90%, 95%, 98%, or even 99%.

The JNK inhibitor sequences as used herein may further comprise or consist of at least one derivative of (native or non-native) amino acid sequences according to SEQ ID NOs: 1-4, 13-20 and 33-100 as defined above. In this context, a “derivative of an (native or non-native) amino acid sequence according to SEQ ID NOs: 1-4, 13-20 and 33-100” is preferably an amino acid sequence derived from any of the sequences according to SEQ ID NOs: 1-4, 13-20 and 33-100, wherein the derivative comprises at least one modified L- or D-amino acid (forming non-natural amino acid(s)), preferably 1 to 20, more preferably 1 to 10, and even more preferably 1 to 5 modified L- or D-amino acids. Derivatives of variants or fragments also fall under the scope of the present invention.

“A modified amino acid” in this respect may be any amino acid which is altered e.g. by different glycosylation in various organisms, by phosphorylation or by labeling specific amino acids. Such a label is then typically selected from the group of labels comprising:
(i) radioactive labels, i.e. radioactive phosphorylation or a radioactive label with sulphur, hydrogen, carbon, nitrogen, etc.;
(ii) colored dyes (e.g. digoxygenin, etc.);
(iii) fluorescent groups (e.g. fluorescein, etc.);
(iv) chemoluminescent groups;
(v) groups for immobilization on a solid phase (e.g. His-tag, biotin, strep-tag, flag-tag, antibodies, antigen, etc.); and
(vi) a combination of labels of two or more of the labels mentioned under (i) to (v).

In the above context, an amino acid sequence having a sequence “sharing a sequence identity” of at least, for example, 95% to a query amino acid sequence of the present invention, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted.

For sequences without exact correspondence, a “% identity” of a first sequence may be determined with respect to a second sequence. In general, these two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length.

Methods for comparing the identity and homology of two or more sequences, particularly as used herein, are well known in the art. Thus for instance, programs available in the Wisconsin Sequence Analysis Package, version 9.1 (Devereux et al., 1984, Nucleic Acids Res. 12, 387-395.), for example the programs BESTFIT and GAP, may be used to determine the % identity between two polynucleotides and the % identity and the % homology between two polypeptide sequences. BESTFIT uses the “local homology” algorithm of (Smith and Waterman (1981), J. Mol. Biol. 147, 195-197.) and finds the best single region of similarity between two sequences. Other programs for determining identity and/or similarity between sequences are also known in the art, for instance the BLAST family of programs (Altschul et al., 1990, J. Mol. Biol. 215, 403-410), accessible through the home page of the NCBI at world wide web site ncbi.nlm.nih.gov) and FASTA (Pearson (1990), Methods Enzymol. 183, 63-98; Pearson and Lipman (1988), Proc. Natl. Acad. Sci. U. S. A 85, 2444-2448.).

JNK-inhibitor sequences as used according to the present invention and as defined above may be obtained or produced by methods well-known in the art, e.g. by chemical synthesis or by genetic engineering methods as discussed below. For example, a peptide corresponding to a portion of an JNK inhibitor sequence as used herein including a desired region of said JNK inhibitor sequence, or that mediates the desired activity in vitro or in vivo, may be synthesized by use of a peptide synthesizer.

JNK inhibitor sequence as used herein and as defined above, may be furthermore be modified by a trafficking sequence, allowing the JNK inhibitor sequence as used herein and as defined above to be transported effectively into the cells. Such modified JNK inhibitor sequence are preferably provided and used as chimeric sequences.

According to a second aspect the present invention therefore provides the use of a chimeric peptide including at least one first domain and at least one second domain, for the preparation of a pharmaceutical composition for preventing and/or treating Mild Cognitive Impairment, in particular Mild Cognitive Impairment due to Alzheimer's Disease, wherein the first domain of the chimeric peptide comprises a trafficking sequence, while the second domain of the chimeric peptide comprises an JNK inhibitor sequence as defined above, preferably of any of sequences according to SEQ ID NO: 1-4, 13-20 and 33-100 or a derivative or a fragment thereof. In other words, the present invention also provides a chimeric peptide comprising at least one first domain and at least one second domain linked by a covalent bond, the first domain comprising a trafficking sequence, and the second domain comprising a JNK inhibitor sequence as defined in any of claims 1 to 9 for use in preventing and/or treating Mild Cognitive Impairment, in particular Mild Cognitive Impairment due to Alzheimer's Disease, as described herein.

Typically, chimeric peptides as used according to the present invention have a length of at least 25 amino acid residues, e.g. 25 to 250 amino acid residues, more preferably 25 to 200 amino acid residues, even more preferably 25 to 150 amino acid residues, 25 to 100 and most preferably amino acid 25 to 50 amino acid residues.

As a first domain the chimeric peptide as used herein preferably comprises a trafficking sequence, which is typically selected from any sequence of amino acids that directs a peptide (in which it is present) to a desired cellular destination. Thus, the trafficking sequence, as used herein, typically directs the peptide across the plasma membrane, e.g. from outside the cell, through the plasma membrane, and into the cytoplasm. Alternatively, or in addition, the trafficking sequence may direct the peptide to a desired location within the cell, e.g. the nucleus, the ribosome, the endoplasmic reticulum (ER), a lysosome, or peroxisome, by e.g. combining two components (e.g. a component for cell permeability and a component for nuclear location) or by one single component having e.g. properties of cell membrane transport and targeted e.g. intranuclear transport. The trafficking sequence may additionally comprise another component, which is capable of binding a cytoplasmic component or any other component or compartment of the cell (e.g. endoplasmic reticulum, mitochondria, gloom apparatus, lysosomal vesicles). Accordingly, e.g. the trafficking sequence of the first domain and the JNK inhibitor sequence of the second domain may be localized in the cytoplasm or any other compartment of the cell. This allows to determine localization of the chimeric peptide in the cell upon uptake.

Preferably, the trafficking sequence (being included in the first domain of the chimeric peptide as used herein) has a length of 5 to 150 amino acid sequences, more preferably a length of 5 to 100 and most preferably a length of from 5 to 50, 5 to 30 or even 5 to 15 amino acids.

More preferably, the trafficking sequence (contained in the first domain of the chimeric peptide as used herein) may occur as a continuous amino acid sequence stretch in the first domain. Alternatively, the trafficking sequence in the first domain may be splitted into two or more fragments, wherein all of these fragments resemble the entire trafficking sequence and may be separated from each other by 1 to 10, preferably 1 to 5 amino acids, provided that the trafficking sequence as such retains its carrier properties as disclosed above. These amino acids separating the fragments of the trafficking sequence may e.g. be selected from amino acid sequences differing from the trafficking sequence. Alternatively, the first domain may contain a trafficking sequence composed of more than one component, each component with its own function for the transport of the cargo JNK inhibitor sequence of the second domain to e.g. a specific cell compartment.

The trafficking sequence as defined above may be composed of L-amino acids, D-amino acids, or a combination of both. Preferably, the trafficking sequence (being included in the first domain of the chimeric peptide as used herein) may comprise at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the JNK trafficking sequences in a blockwise, a non-blockwise or in an alternate manner.

According to one alternative embodiment, the trafficking sequence of the chimeric peptide as used herein may be exclusively composed of L-amino acids. More preferably, the trafficking sequence of the chimeric peptide as used herein comprises or consists of at least one “native” trafficking sequence as defined above. In this context, the term “native” is referred to non-altered trafficking sequences, entirely composed of L-amino acids.

According to another alternative embodiment the trafficking sequence of the chimeric peptide as used herein may be exclusively composed of D-amino acids. More preferably, the trafficking sequence of the chimeric peptide as used herein may comprise a D retro-inverso peptide of the sequences as presented above.

The trafficking sequence of the first domain of the chimeric peptide as used herein may be obtained from naturally occurring sources or can be produced by using genetic engineering techniques or chemical synthesis (see e.g. Sambrook, J., Fritsch, E. F., Maniatis, T. (1989) Molecular cloning: A laboratory manual. 2nd edition. Cold Spring Harbor Laboratory Press, Cold Spring Harbor, N.Y.).

Sources for the trafficking sequence of the first domain may be employed including, e.g. native proteins such as e.g. the TAT protein (e.g. as described in U.S. Pat. Nos. 5,804,604 and 5,674,980, each of these references being incorporated herein by reference), VP22 (described in e.g. WO 97/05265; Elliott and O'Hare, Cell 88: 223-233 (1997)), non-viral proteins (Jackson et al, Proc. Natl. Acad. Sci. USA 89: 10691-10695 (1992)), trafficking sequences derived from Antennapedia (e.g. the antennapedia carrier sequence) or from basic peptides, e.g. peptides having a length of 5 to 15 amino acids, preferably 10 to 12 amino acids and comprising at least 80%, more preferably 85% or even 90% basic amino acids, such as e.g. arginine, lysine and/or histidine. Furthermore, variants, fragments and derivatives of one of the native proteins used as trafficking sequences are disclosed herewith. With regard to variants, fragments and derivatives it is referred to the definition given above for JNK inhibitor sequences as used herein. Variants, fragments as well as derivatives are correspondingly defined as set forth above for JNK inhibitor sequences as used herein. Particularly, in the context of the trafficking sequence, a variant or fragment or derivative may be defined as a sequence sharing a sequence identity with one of the native proteins used as trafficking sequences as defined above of at least about 30%, 50%, 70%, 80%, 90%, 95%, 98%, or even 99%.

In a preferred embodiment of the chimeric peptide as used herein, the trafficking sequence of the first domain comprises or consists of a sequence derived from the human immunodeficiency virus (HIV)1 TAT protein, particularly some or all of the 86 amino acids that make up the TAT protein.

For a trafficking sequence (being included in the first domain of the chimeric peptide as used herein), partial sequences of the full-length TAT protein may be used forming a functionally effective fragment of a TAT protein, i.e. a TAT peptide that includes the region that mediates entry and uptake into cells. As to whether such a sequence is a functionally effective fragment of the TAT protein can be determined using known techniques (see e.g. Franked et al., Proc. Natl. Acad. Sci, USA 86: 7397-7401 (1989)). Thus, the trafficking sequence in the first domain of the chimeric peptide as used herein may be derived from a functionally effective fragment or portion of a TAT protein sequence that comprises less than 86 amino acids, and which exhibits uptake into cells, and optionally the uptake into the cell nucleus. More preferably, partial sequences (fragments) of TAT to be used as carrier to mediate permeation of the chimeric peptide across the cell membrane, are intended to comprise the basic region (amino acids 48 to 57 or 49 to 57) of full-length TAT.

According to a more preferred embodiment, the trafficking sequence (being included in the first domain of the chimeric peptide as used herein) may comprise or consist of an amino acid sequence containing TAT residues 48-57 or 49 to 57, and most preferably a generic TAT sequence NH₂-Xₙ^{b}-RKKRRQRRR-Xₙ^{b}-COOH (L-generic-TAT (s)) [SEQ ID NO: 7] and/or XXXXRKKRRQ RRRXXXX (L-generic-TAT) [SEQ ID NO: 21], wherein X or Xₙ^{b} is as defined above. Furthermore, the number of “Xₙ^{b}” residues in SEQ ID NOs:8 is not limited to the one depicted, and may vary as described above. Alternatively, the trafficking sequence being included in the first domain of the chimeric peptide as used herein may comprise or consist of a peptide containing e.g. the amino acid sequence NH₂-GRKKRRQRRR-COOH (L-TAT) [SEQ ID NO: 5].

According to another more preferred embodiment the trafficking sequence (being included in the first domain of the chimeric peptide as used herein) may comprise a D retro-inverso peptide of the sequences as presented above, i.e. the D retro-inverso sequence of the generic TAT sequence having the sequence NH₂-Xₙ^{b}-RRRQRRKKR-Xₙ^{b}-COOH (D-generic-TAT (s)) [SEQ ID NO: 8] and/or XXXXRRRQRRKKRXXXX (D-generic-TAT) [SEQ ID NO: 22]. Also here, Xₙ^{b} is as defined above (preferably representing D amino acids). Furthermore, the number of “Xₙ^{b}” residues in SEQ ID NOs:8 is not limited to the one depicted, and may vary as described above. Most preferably, the trafficking sequence as used herein may comprise the D retro-inverso sequence NH₂-RRRQRRKKRG-COOH (D-TAT) [SEQ ID NO: 6].

According to another embodiment the trafficking sequence being included in the first domain of the chimeric peptide as used herein may comprise or consist of variants of the trafficking sequences as defined above. A “variant of a trafficking sequence” is preferably a sequence derived from a trafficking sequence as defined above, wherein the variant comprises a modification, for example, addition, (internal) deletion (leading to fragments) and/or substitution of at least one amino acid present in the trafficking sequence as defined above. Such (a) modification(s) typically comprise(s) 1 to 20, preferably 1 to 10 and more preferably 1 to 5 substitutions, additions and/or deletions of amino acids. Furthermore, the variant preferably exhibits a sequence identity with the trafficking sequence as defined above, more preferably with any of SEQ ID NOs: 5 to 8 or 21-22, of at least about 30%, 50%, 70%, 80%, 90%, 95%, 98% or even 99%.

Preferably, such a modification of the trafficking sequence being included in the first domain of the chimeric peptide as used herein leads to a trafficking sequence with increased or decreased stability. Alternatively, variants of the trafficking sequence can be designed to modulate intracellular localization of the chimeric peptide as used herein. When added exogenously, such variants as defined above are typically designed such that the ability of the trafficking sequence to enter cells is retained (i.e. the uptake of the variant of the trafficking sequence into the cell is substantially similar to that of the native protein used a trafficking sequence). For example, alteration of the basic region thought to be important for nuclear localization (see e.g. Dang and Lee, J. Biol. Chem. 264: 18019-18023 (1989); Hauber et al., J. Virol. 63: 1181-1187 (1989); et al., J. Virol. 63: 1-8 (1989)) can result in a cytoplasmic location or partially cytoplasmic location of the trafficking sequence, and therefore, of the JNK inhibitor sequence as component of the chimeric peptide as used herein. Additional to the above, further modifications may be introduced into the variant, e.g. by linking e.g. cholesterol or other lipid moieties to the trafficking sequence to produce a trafficking sequence having increased membrane solubility. Any of the above disclosed variants of the trafficking sequences being included in the first domain of the chimeric peptide as used herein can be produced using techniques typically known to a skilled person (see e.g. Sambrook, J., Fritsch, E. F., Maniatis, T. (1989) Molecular cloning: A laboratory manual. 2nd edition. Cold Spring Harbor Laboratory, Cold Spring Harbor, N.Y.)

As a second domain the chimeric peptide as used herein typically comprises an JNK inhibitor sequence, selected from any of the JNK inhibitor sequences as defined above, including variants, fragments and/or derivatives of these JNK inhibitor sequences.

Both domains, i.e. the first and the second domain(s), of the chimeric peptide as used herein, may be linked such as to form a functional unit. Any method for linking the first and second domain(s) as generally known in the art may be applied.

According to one embodiment, the first and the second domain(s) of the chimeric peptide as used herein are preferably linked by a covalent bond. A covalent bond, as defined herein, may be e.g. a peptide bond, which may be obtained by expressing the chimeric peptide as defined above as a fusion protein. Fusion proteins, as described herein, can be formed and used in ways analogous to or readily adaptable from standard recombinant DNA techniques, as described below. However, both domains may also be linked via side chains or may be linked by a chemical linker moiety.

The first and/or second domains of the chimeric peptide as used herein may occur in one or more copies in said chimeric peptide. If both domains are present in a single copy, the first domain may be linked either to the N-terminal or the C-terminal end of the second domain.

If present in multiple copies, the first and second domain(s) may be arranged in any possible order. E.g. the first domain can be present in the chimeric peptide as used herein in a multiple copy number, e.g. in two, three or more copies, which are preferably arranged in consecutive order. Then, the second domain may be present in a single copy occurring at the N- or C-terminus of the sequence comprising the first domain. Alternatively, the second domain may be present in a multiple copy number, e.g. in two, three or more copies, and the first domain may be present in a single copy. According to both alternatives, first and second domain(s) can take any place in a consecutive arrangement. Exemplary arrangements are shown in the following: e.g. first domain-first domain-first domain-second domain; first domain-first domain-second domain-first domain; first domain-second domain-first domain-first domain; or e.g. second domain-first domain-first domain-first domain. It is well understood for a skilled person that these examples are for illustration purposes only and shall not limit the scope of the invention thereto. Thus, the number of copies and the arrangement may be varied as defined initially.

Preferably, the first and second domain(s) may be directly linked with each other without any linker. Alternatively, they may be linked with each other via a linker sequence comprising 1 to 10, preferably 1 to 5 amino acids. Amino acids forming the linker sequence are preferably selected from glycine or proline as amino acid residues. More preferably, the first and second domain(s) may be separated by each other by a hinge of two, three or more proline residues between the first and second domain(s).

The chimeric peptide as defined above and as used herein, comprising at least one first and at least one second domain, may be composed of L-amino acids, D-amino acids, or a combination of both. Therein, each domain (as well as the linkers used) may be composed of L-amino acids, D-amino acids, or a combination of both (e.g. D-TAT and L-IB1(s) or L-TAT and D-IB1(s), etc.). Preferably, the chimeric peptide as used herein may comprise at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the chimeric peptide as used herein in a blockwise, a non-blockwise or in an alternate manner.

According to a specific embodiment the chimeric peptide as used herein comprises or consists of the L-amino acid chimeric peptides according to the generic L-TAT-IB peptide NH₂-Xₙ^{b}-RKKRRQRRR-Xₙ^{b}-Xₙ^{a}-RPTTLXLXXXXXXXQD-Xₙ^{b}-COOH (L-TAT-IB (generic) (s)) [SEQ ID NO: 10], wherein X, Xₙ^{a} and Xₙ^{b} are preferably as defined above. More preferably, the chimeric peptide as used herein comprises or consists of the L-amino acid chimeric peptide NH₂-GRKKRRQRRRPPRPKRPTTLNLFPQVPRSQD-COOH (L-TAT-IB1 (s)) [SEQ ID NO: 9]. Alternatively or additionally, the chimeric peptide as used herein comprises or consists of the L-amino acid chimeric peptide sequence GRKKRRQRRR PPDTYRPKRP TTLNLFPQVP RSQDT (L-TAT-IB1) [SEQ ID NO: 23], or XXXXXXXRKK RRQRRRXXXX XXXXRPTTLX LXXXXXXXQD S/TX (L-TAT-IB generic) [SEQ ID NO: 24], wherein X is preferably also as defined above, or the chimeric peptide as used herein comprises or consists of the L-amino acid chimeric peptide sequence RKKRRQRRRPPRPKRPTTLNLFPQVPRSQD (L-TAT-IB1(s1)) [SEQ ID NO: 27], GRKKRRQRRRXₙ^{c}CRPKRPTTLNLFPQVPRSQD (L-TAT-IB1(s2)) [SEQ ID NO: 28], or RKKRRQRRRXₙ^{c}RPKRPTTLNLFPQVPRSQD (L-TAT-IB1(s3)) [SEQ ID NO: 29]. In this context, each X typically represents an amino acid residue as defined above, more preferably Xₙ^{c} represents a contiguous stretch of peptide residues, each X independently selected from each other from glycine or proline, e.g. a monotonic glycine stretch or a monotonic proline stretch, wherein n (the number of repetitions of Xₙ^{c}) is typically 0-5, 5-10, 10-15, 15-20, 20-30 or even more, preferably 0-5 or 5-10. Xₙ^{c} may represent either D or L amino acids.

According to an alternative specific embodiment the chimeric peptide as used herein comprises or consists of D-amino acid chimeric peptides of the above disclosed L-amino acid chimeric peptides. Exemplary D retro-inverso chimeric peptides according to the present invention are e.g. the generic D-TAT-IB peptide NH₂-Xₙ^{b}-DQXXXXXXXLXLTTPR-Xₙ^{a}-Xₙ^{b}-RRRQRRKKR-Xₙ^{b}-COOH (D-TAT-IB (generic) (s)) [SEQ ID NO: 12]. Herein, X, Xₙ^{a} and Xₙ^{b} are preferably as defined above (preferably representing D amino acids). More preferably, the chimeric peptide as used herein comprises or consists of D-amino acid chimeric peptides according to the TAT-IB1 peptide NH₂-DQSRPVQPFLNLTTPRKPRPPRRRQRRKKRG-COOH (D-TAT-IB1(s)) [SEQ ID NO: 11]. Alternatively or additionally, the chimeric peptide as used herein comprises or consists of the D-amino acid chimeric peptide sequence TDQSRPVQPFLNLTTPRKPRYTDPPRRRQRRKKRG (D-TAT-IB1) [SEQ ID NO: 25], or XT/SDQXXXXXXXLXLTTPRXXXXXXXXRRRQRRKKRXXXXXXX (D-TAT-IB generic) [SEQ ID NO: 26], wherein X is preferably also as defined above, or the chimeric peptide as used herein comprises or consists of the D-amino acid chimeric peptide sequence DQSRPVQPFLNLTTPRKPRPPRRRQRRKKR (D-TAT-IB1 (s1)) [SEQ ID NO: 30], DQSRPVQPFLNLTTPRKPRXₙ^{c}RRRQRRKKRG (D-TAT-IB1(s2)) [SEQ ID NO: 31], or DQSRPVQPFLNLTTPRKPRXₙ^{c}RRRQRRKKR (D-TAT-IB1(s3)) [SEQ ID NO: 32]. Xₙ^{c} may be as defined above.

The first and second domain(s) of the chimeric peptide as defined above may be linked to each other by chemical or biochemical coupling carried out in any suitable manner known in the art, e.g. by establishing a peptide bond between the first and the second domain(s) e.g. by expressing the first and second domain(s) as a fusion protein, or e.g. by crosslinking the first and second domain(s) of the chimeric peptide as defined above.

Many known methods suitable for chemical crosslinking of the first and second domain(s) of the chimeric peptide as defined above are non-specific, i.e. they do not direct the point of coupling to any particular site on the transport polypeptide or cargo macromolecule. As a result, use of non-specific crosslinking agents may attack functional sites or sterically block active sites, rendering the conjugated proteins biologically inactive. Thus, preferably such crosslinking methods are used, which allow a more specific coupling of the first and second domain(s).

In this context, one way to increasing coupling specificity is a direct chemical coupling to a functional group present only once or a few times in one or both of the first and second domain(s) to be crosslinked. For example, cysteine, which is the only protein amino acid containing a thiol group, occurs in many proteins only a few times. Also, for example, if a polypeptide contains no lysine residues, a crosslinking reagent specific for primary amines will be selective for the amino terminus of that polypeptide. Successful utilization of this approach to increase coupling specificity requires that the polypeptide have the suitably rare and reactive residues in areas of the molecule that may be altered without loss of the molecule's biological activity. Cysteine residues may be replaced when they occur in parts of a polypeptide sequence where their participation in a crosslinking reaction would otherwise likely interfere with biological activity. When a cysteine residue is replaced, it is typically desirable to minimize resulting changes in polypeptide folding. Changes in polypeptide folding are minimized when the replacement is chemically and sterically similar to cysteine. For these reasons, serine is preferred as a replacement for cysteine. As demonstrated in the examples below, a cysteine residue may be introduced into a polypeptide's amino acid sequence for crosslinking purposes. When a cysteine residue is introduced, introduction at or near the amino or carboxy terminus is preferred. Conventional methods are available for such amino acid sequence modifications, wherein the polypeptide of interest is produced by chemical synthesis or via expression of recombinant DNA.

Coupling of the first and second domain(s) of the chimeric peptide as defined above and used herein can also be accomplished via a coupling or conjugating agent. There are several intermolecular crosslinking reagents which can be utilized (see for example, Means and Feeney, CHEMICAL MODIFICATION OF PROTEINS, Holden-Day, 1974, pp. 39-43). Among these reagents are, for example, N-succinimidyl 3-(2-pyridyldithio) propionate (SPDP) or N,N′-(1,3-phenylene) bismaleimide (both of which are highly specific for sulfhydryl groups and form irreversible linkages); N, N′-ethylene-bis-(iodoacetamide) or other such reagent having 6 to 11 carbon methylene bridges (which are relatively specific for sulfhydryl groups); and 1,5-difluoro-2,4-dinitrobenzene (which forms irreversible linkages with amino and tyrosine groups). Other crosslinking reagents useful for this purpose include: p,p′-difluoro-m, m′-dinitrodiphenylsulfone which forms irreversible crosslinkages with amino and phenolic groups); dimethyl adipimidate (which is specific for amino groups); phenol-1,4 disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Crosslinking reagents used for crosslinking the first and second domain(s) of the chimeric peptide as defined above may be homobifunctional, i.e. having two functional groups that undergo the same reaction. A preferred homobifunctional crosslinking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible crosslinking of polypeptides that contain cysteine residues.

Crosslinking reagents used for crosslinking the first and second domain(s) of the chimeric peptide as defined above may also be heterobifunctional. Heterobifunctional crosslinking agents have two different functional groups, for example an amine-reactive group and a thiol-reactive group, that will crosslink two proteins having free amines and thiols, respectively. Examples of heterobifunctional crosslinking agents are succinimidyl 4-(N-maleimidomethyl)cyclohexane-1-carboxylate (“SMCC”), m-maleimidobenzoyl-N-hydroxysuccinimide ester (“MBS”), and succinimide 4-(p-maleimidophenyl)butyrate (“SMPB”), an extended chain analog of MBS. The succinimidyl group of these crosslinkers reacts with a primary amine, and the thiol-reactive maleimide forms a covalent bond with the thiol of a cysteine residue.

Crosslinking reagents suitable for crosslinking the first and second domain(s) of the chimeric peptide as defined above often have low solubility in water. A hydrophilic moiety, such as a sulfonate group, may thus be added to the crosslinking reagent to improve its water solubility. In this respect, Sulfo-MBS and Sulfo-SMCC are examples of crosslinking reagents modified for water solubility, which may be used according to the present invention.

Likewise, many crosslinking reagents yield a conjugate that is essentially non-cleavable under cellular conditions. However, some crosslinking reagents particularly suitable for crosslinking the first and second domain(s) of the chimeric peptide as defined above contain a covalent bond, such as a disulfide, that is cleavable under cellular conditions. For example, Traut's reagent, dithiobis(succinimidylpropionate) (“DSP”), and N-succinimidyl 3-(2-pyridyldithio)propionate (“SPDP”) are well-known cleavable crosslinkers. The use of a cleavable crosslinking reagent permits the cargo moiety to separate from the transport polypeptide after delivery into the target cell. Direct disulfide linkage may also be useful.

Numerous crosslinking reagents, including the ones discussed above, are commercially available. Detailed instructions for their use are readily available from the commercial suppliers. A general reference on protein crosslinking and conjugate preparation is: Wong, CHEMISTRY OF PROTEIN CONJUGATION AND CROSSLINKING, CRC Press (1991).

Chemical crosslinking of the first and second domain(s) of the chimeric peptide as defined above may include the use of spacer arms. Spacer arms provide intramolecular flexibility or adjust intramolecular distances between conjugated moieties and thereby may help preserve biological activity. A spacer arm may be in the form of a polypeptide moiety that includes spacer amino acids, e.g. proline. Alternatively, a spacer arm may be part of the crosslinking reagent, such as in “long-chain SPDP” (Pierce Chem. Co., Rockford, Ill., cat. No. 21651 H).

Preferably, any of the peptides disclosed herein, in particular the JNK inhibitor, the trafficking sequence and the chimeric peptide as disclosed herein, preferably the JNK inhibitor according to SEQ ID NO: 11, may have a modification at one or both of their termini, i.e. either at the C- or at the N-terminus or at both. The C-Terminus may preferably be modified by an amide modification, whereas the N-terminus may be modified by any suitable NH2-protection group, such as e.g. acylation. More preferably, the JNK inhibitor and the chimeric peptide as disclosed herein, preferably the JNK inhibitor according to SEQ ID NO: 11, is modified by an amide modification at the C-terminus.

Preferably, in the chimeric peptide as described herein, in particular the chimeric peptide comprising (a) an amino acid sequence which is at least 70%, preferably at least 80%, more preferably at least 90%, even more preferably at least 95%, and most preferably at least 98% identical to SEQ ID NO: 11 or (b) an amino acid sequence according to SEQ ID NO: 11,
(i) the C-terminus of the chimeric peptide is modified by an amide modification; and/or
(ii) the N-terminus of the chimeric peptide is modified by an NH₂-protection group, such as acylation.

It is also preferred that any of the peptides disclosed herein, in particular the JNK inhibitor, the trafficking sequence (e.g. of the chimeric peptide) and the chimeric peptide as disclosed herein, preferably the JNK inhibitor according to SEQ ID NO: 11, may be deleted at their N- and/or C-terminus by 1, 2 or 3 amino acids. For example, in a chimeric peptide according to the present invention each domain, i.e. the JNK-inhibitor and the trafficking sequence domain, may be deleted at their N- and/or C-terminus by 1, 2 or 3 amino acids and/or the chimeric peptide according to the present invention may be deleted at its N- and/or C-terminus by 1, 2 or 3 amino acids. More preferably, the inventive chimeric peptide comprises or consists of a D-amino acid chimeric peptide according to the TAT-IB1 peptide [NH₂-DQSRPVQPFLNLTTPRKPRPPRRRQRRKKRG-COOH, SEQ ID NO: 11] and the linking portion of the first and second domain (instead of PP) may be composed of -Xₙ^{a}-Xₙ^{b}-, which are as defined above. In particular, the second domain(s) of SEQ ID NO: 11, eventually with -Xₙ^{a}-Xₙ^{b}- instead of (PP), may be deleted at their N- and/or C-terminus by 1, 2 or 3 amino acids. In another preferred embodiment, the first domain of SEQ ID NO: 11 may be deleted at its N- and or C-terminus by 1, 2 or 3 amino acids. This/these deletion/s may be combined with the deletion/s disclosed for the amino acid residues of the termini of the second domain. Again, the shorter the peptides are, the less their (unspecific) cell toxicity. However, the peptides must retain their biological function, i.e. their cell membrane permeability (first domain) and their JNK inhibitory function (second domain).

Furthermore, variants, fragments or derivatives of one of the above disclosed chimeric peptides may be used herein. With regard to fragments and variants it is generally referred to the definition given above for JNK inhibitor sequences.

Particularly, in the context of the present invention, a “variant of a chimeric peptide” is preferably a sequence derived from any of the sequences according to SEQ ID NOs: 9 to 12 and 23 to 32, wherein the chimeric variant comprises amino acid alterations of the chimeric peptides according to SEQ ID NOs: 9 to 12 and 23 to 32 as used herein. Such alterations typically comprise 1 to 20, preferably 1 to 10 and more preferably 1 to 5 substitutions, additions and/or deletions (leading to fragments) of amino acids according to SEQ ID NOs: 9 to 12 and 23 to 32, wherein the altered chimeric peptide as used herein exhibits a sequence identity with any of the sequences according to SEQ ID NOs: 9-12 and 23 to 32 of at least about 30%, 50%, 70%, 80%, or 95%, 98%, or even 99%.

Preferably, the chimeric peptide consists of or comprises an amino acid sequence having at least 70%, preferably at least 80%, more preferably at least 90%, even more preferably at least 95%, and most preferably at least 98% sequence identity to SEQ ID NO: 9 or 11. More preferably, the chimeric peptide consists of or comprises the amino acid sequence of SEQ ID NO: 9 or 11. It is particularly preferred that the chimeric peptide consists of or comprises
(i) the amino acid sequence of SEQ ID NO: 11; or
(ii) an amino acid sequence having at least 70%, preferably at least 80%, more preferably at least 90%, even more preferably at least 95%, and most preferably at least 98% sequence identity to SEQ ID NO: 11.

Preferably, the above described variants retain the biological activity of the first and the second domain as contained in the chimeric peptide as used herein, i.e. the trafficking activity of the first domain as disclosed above and the activity of the second domain for binding JNK and/or inhibiting the activation of at least one JNK activated transcription factor.

Accordingly, the chimeric peptide as used herein also comprises fragments of the afore disclosed chimeric peptides, particularly of the chimeric peptide sequences according to any of SEQ ID NOs: 9 to 12 and 23 to 32. Thus, in the context of the present invention, a “fragment of the chimeric peptide” is preferably a sequence derived any of the sequences according to SEQ ID NOs: 9 to 12 and 23 to 32, wherein the fragment comprises at least 4 contiguous amino acids of any of SEQ ID NOs: 9 to 12 and 23 to 32. This fragment preferably comprises a length which is sufficient to allow specific recognition of an epitope from any of these sequences and to transport the sequence into the cells, the nucleus or a further preferred location. Even more preferably, the fragment comprises 4 to 18, 4 to 15, or most preferably 4 to 10 contiguous amino acids of any of SEQ ID NOs: 9 to 12 and 23 to 32. Fragments of the chimeric peptide as used herein further may be defined as a sequence sharing a sequence identity with any of the sequences according to any of SEQ ID NOs: 9 to 12 and 23 to 32 of at least about 30%, 50%, 70%, 80%, or 95%, 98%, or even 99%.

Finally, the chimeric peptide as used herein also comprises derivatives of the afore disclosed chimeric peptides, particularly of the chimeric peptide sequences according to any of SEQ ID NOs: 9 to 12 and 23 to 32.

In a further aspect, the present invention provides combination therapies, such as a combination of
(a) the JNK inhibitor sequence as described herein or the chimeric peptide as described herein; and
(b) a PKR inhibitor
   for use in preventing and/or treating Mild Cognitive Impairment, in particular Mild Cognitive Impairment due to Alzheimer's Disease.

A PKR inhibitor is an inhibitor of double-stranded RNA-dependent protein kinase (PKR). Preferred examples of PKR inhibitors include C16 (also referred to as PKRi), 2-arninopurine (2-Aβ), and peptide PKR inhibitors, such as the peptides P1 and P2 described in M. J. Du et al., Selection of peptide inhibitors for double-stranded RNA-dependent protein kinase PKR, Biochemistry (Mosc.) 2013 November; 78(11):1254-62. Du et al., 2013 also provide a method of how to identify PKR peptide inhibitors. Peptide PKR inhibitors are more preferred and a particularly preferred peptide PKR inhibitor is “SC1481” provided by PolyPeptide Group.

Preferably, the above described combination further comprises
(c) an amyloid lowering agent; and/or
(d) a glucocorticoid.

Amyloid lowering agents include p3-secretase (BACE1) inhibitors, γ-secretase inhibitors (GSI) and modulators (GSM). Examples of such amyloid lowering agents, which are currently in clinical trials may be retrieved from Vassar R. (2014) BACE1 inhibitor drugs in clinical trials for Alzheimer's disease. Alzheimers Res Ther.; 6(9):89 or from Jia Q, Deng Y, Qing H (2014) Potential therapeutic strategies for Alzheimer's disease targeting or beyond p3-amyloid: insights from clinical trials. Biomed Res Int. 2014; 2014:837157; for example Pioglitazone, CTS-21166, MK8931, LY2886721, AZD3293, E2609, NIC5-15, Begacestat, CHF 5074, EVP-0962, Atorvastatin, Simvastatin, Etazolate, Epigallocatechin-3-gallate (EGCg), Scyllo-inositol (ELND005/AZD103), Tramiprosate (3 APS), PBT2, Affitope AD02, and Affitope AD03. Further preferred amyloid lowering agents are those described by M. S. Wolfe, Amyloid lowering agents, BMC Neurosci. 2008; 9(Suppl 2): S4.

Preferred examples of glucocorticoids include hydrocortisone, prednisone, prednisolone, methylprednisolone, dexamethasone, betamethasone, triamcinolone, beclomethasone, fludrocortisone, and deoxycorticosetrone. Dexamethasone, hydrocortisone, prednisone, prednisolone, and methylprednisolone are particularly preferred.

The present invention also provides a further combination therapy, namely a combination of
(a) the JNK inhibitor sequence as described herein or the chimeric peptide as described herein; and
(b) an amyloid lowering agent
   for use in preventing and/or treating Mild Cognitive Impairment, in particular Mild Cognitive Impairment due to Alzheimer's Disease.

Preferably, this combination further comprises
(c) a PKR inhibitor; and/or
(d) a glucocorticoid.

Thereby, the amyloid lowering agent, the PKR inhibitor and the glucocorticoid may be selected as described above.

In general in the combination therapies described herein the different components may be administered in separate or in the same pharmaceutical compositions. If the combination therapy comprises more than two components, it is also possible that two (or more) of the components are comprised in the same pharmaceutical composition, whereas at least one further component is administered in a separate pharmaceutical composition. In general, separate pharmaceutical compositions for the active components to be combined are preferred for better individual dosing, however for convenience also a single pharmaceutical composition comprising the active components to be combined is conceivable.

In the case of separate pharmaceutical compositions for the active components to be combined, the JNK inhibitor or the chimeric peptide according to the present invention may be administered before, during (concomitant or overlapping administration) or after administration of the other active component(s), for example the PKR inhibitor, the amyloid lowering agent and/or the glucocorticoid. Preferably, the JNK inhibitor sequence or the chimeric peptide is administered before or after the PKR inhibitor, the amyloid lowering agent and/or the glucocorticoid.

Administration of the JNK inhibitor sequence or the chimeric peptide “before” the administration of the PKR inhibitor, the amyloid lowering agent and/or the glucocorticoid preferably means that the administration of the JNK inhibitor sequence or the chimeric peptide is finished within 24 h, more preferably within 12 h, even more preferably within 3 h, particularly preferably within 1 h and most preferably within 30 min before the administration of the PKR inhibitor, the amyloid lowering agent and/or the glucocorticoid starts. Administration “after” the administration of the PKR inhibitor, the amyloid lowering agent and/or the glucocorticoid preferably means within 24 h, more preferably within 12 h, even more preferably within 3 h, particularly preferably within 1 h and most preferably within 30 min after the administration of the PKR inhibitor, the amyloid lowering agent and/or the glucocorticoid is finished.

Moreover, the JNK inhibitor sequence or the chimeric peptide may be administered via the same route of administration or via a distinct route of administration as the PKR inhibitor, the amyloid lowering agent and/or the glucocorticoid. Preferred routes of administration are described below, in particular in the context of the pharmaceutical compositions. Preferred embodiments described for pharmaceutical compositions apply also in the context of the combination therapy.

The present invention additionally refers to the use of nucleic acid sequences encoding JNK inhibitor sequences as defined above, chimeric peptides or their fragments, variants or derivatives, all as defined above, for the preparation of a pharmaceutical composition for preventing and/or treating Mild Cognitive Impairment, in particular Mild Cognitive Impairment due to Alzheimer's Disease, as defined above in a subject. In other words, the present invention also provides an isolated nucleic acid encoding a JNK inhibitor sequence as described herein or a chimeric peptide as described herein for use in preventing and/or treating Mild Cognitive Impairment, in particular Mild Cognitive Impairment due to Alzheimer's Disease. A preferable suitable nucleic acid encoding a JNK inhibitor sequence as used herein is typically chosen from human IB1 nucleic acid (GenBank Accession No. (AF074091), rat IB1 nucleic acid (GenBank Accession No. AF 108959), or human IB2 (GenBank Accession No AF218778) or from any nucleic acid sequence encoding any of the sequences as defined above, i.e. any sequence according to SEQ ID NO: 1-26.

Nucleic acids encoding the JNK inhibitor sequences as used herein or chimeric peptides as used herein may be obtained by any method known in the art (e.g. by PCR amplification using synthetic primers hybridizable to the 3′- and 5′-termini of the sequence and/or by cloning from a cDNA or genomic library using an oligonucleotide sequence specific for the given gene sequence).

Additionally, nucleic acid sequences are disclosed herein as well, which hybridize under stringent conditions with the appropriate strand coding for a (native) JNK inhibitor sequence or chimeric peptide as defined above. Preferably, such nucleic acid sequences comprise at least 6 (contiguous) nucleic acids, which have a length sufficient to allow for specific hybridization. More preferably, such nucleic acid sequences comprise 6 to 38, even more preferably 6 to 30, and most preferably 6 to 20 or 6 to 10 (contiguous) nucleic acids.

“Stringent conditions” are sequence dependent and will be different under different circumstances. Generally, stringent conditions can be selected to be about 5° C. lower than the thermal melting point (TM) for the specific sequence at a defined ionic strength and pH. The TM is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may affect the stringency of hybridization (including, among others, base composition and size of the complementary strands), the presence of organic solvents and the extent of base mismatching, the combination of parameters is more important than the absolute measure of any one.

“High stringency conditions” may comprise the following, e.g. Step 1: Filters containing DNA are pretreated for 8 hours to overnight at 65° C. in buffer composed of 6*SSC, 50 mM Tris-HCl (pH 7.5), 1 mM EDTA, 0.02% PVP, 0.02% Ficoll, 0.02% BSA, and 500 μg/ml denatured salmon sperm DNA. Step 2: Filters are hybridized for 48 hours at 65° C. in the above prehybridization mixture to which is added 100 mg/ml denatured salmon sperm DNA and 5-20*10⁶ cpm of ³²P-labeled probe. Step 3: Filters are washed for 1 hour at 37° C. in a solution containing 2*SSC, 0.01% PVP, 0.01% Ficoll, and 0.01% BSA. This is followed by a wash in 0.1*SSC at 50° C. for 45 minutes. Step 4: Filters are autoradiographed. Other conditions of high stringency that may be used are well known in the art (see e.g. Ausubel et al., (eds.), 1993, Current Protocols in Molecular Biology, John Wiley and Sons, NY; and Kriegler, 1990, Gene Transfer and Expression, a Laboratory Manual, Stockton Press, N.Y.).

“Moderate stringency conditions” can include the following: Step 1: Filters containing DNA are pretreated for 6 hours at 55° C. in a solution containing 6*SSC, 5*Denhardt's solution, 0.5% SDS and 100 mg/ml denatured salmon sperm DNA. Step 2: Filters are hybridized for 18-20 hours at 55° C. in the same solution with 5-20*10⁶ cpm ³²P-labeled probe added. Step 3: Filters are washed at 37° C. for 1 hour in a solution containing 2*SSC, 0.1% SDS, then washed twice for 30 minutes at 60° C. in a solution containing 1*SSC and 0.1% SDS. Step 4: Filters are blotted dry and exposed for autoradiography. Other conditions of moderate stringency that may be used are well-known in the art (see e.g. Ausubel et al., (eds.), 1993, Current Protocols in Molecular Biology, John Wiley and Sons, NY; and Kriegler, 1990, Gene Transfer and Expression, a Laboratory Manual, Stockton Press, N.Y.).

Finally, “low stringency conditions” can include: Step 1: Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Step 2: Filters are hybridized for 18-20 hours at 40° C. in the same solution with the addition of 0.02% PVP, 0.02% Ficoll, 0.2% BSA, 100 μg/ml salmon sperm DNA, 10% (wt/vol) dextran sulfate, and 5-20×106 cpm ³²P-labeled probe. Step 3: Filters are washed for 1.5 hours at 55 C in a solution containing 2×SSC, 25 mM Tris-HCl (pH 7.4), 5 mM EDTA, and 0.1% SDS. The wash solution is replaced with fresh solution and incubated an additional 1.5 hours at 60° C. Step 4: Filters are blotted dry and exposed for autoradiography. If necessary, filters are washed for a third time at 65-68° C. and reexposed to film. Other conditions of low stringency that may be used are well known in the art (e.g. as employed for cross-species hybridizations). See e.g. Ausubel et al., (eds.), 1993, CURRENT PROTOCOLS IN MOLECULAR BIOLOGY, John Wiley and Sons, NY; and Kriegler, 1990, GENE TRANSFER AND EXPRESSION, A LABORATORY MANUAL, Stockton Press, N.Y.

The nucleic acid sequences as defined above according to the present invention can be used to express peptides, i.e. an JNK inhibitor sequence as used herein or an chimeric peptide as used herein for analysis, characterization or therapeutic use; as markers for tissues in which the corresponding peptides (as used herein) are preferentially expressed (either constitutively or at a particular stage of tissue differentiation or development or in disease states). Other uses for these nucleic acids include, e.g. molecular weight markers in gel electrophoresis-based analysis of nucleic acids.

According to a further embodiment of the present invention, expression vectors may be used for the above purposes for recombinant expression of one or more JNK inhibitor sequences and/or chimeric peptides as defined above. In other words, the present invention also provides vector comprising the nucleic acid as described above for use in (the preparation of a medicament for) preventing and/or treating Mild Cognitive Impairment, in particular Mild Cognitive Impairment due to Alzheimer's Disease. The term “expression vector” is used herein to designate either circular or linear DNA or RNA, which is either double-stranded or single-stranded. It further comprises at least one nucleic acid as defined above to be transferred into a host cell or into a unicellular or multicellular host organism. The expression vector as used herein preferably comprises a nucleic acid as defined above encoding the JNK inhibitor sequence as used herein or a fragment or a variant thereof, or the chimeric peptide as used herein, or a fragment or a variant thereof. Additionally, an expression vector according to the present invention preferably comprises appropriate elements for supporting expression including various regulatory elements, such as enhancers/promoters from viral, bacterial, plant, mammalian, and other eukaryotic sources that drive expression of the inserted polynucleotide in host cells, such as insulators, boundary elements, LCRs (e.g. described by Blackwood and Kadonaga (1998), Science 281, 61-63) or matrix/scaffold attachment regions (e.g. described by Li, Harju and Peterson, (1999), Trends Genet. 15, 403-408). In some embodiments, the regulatory elements are heterologous (i.e. not the native gene promoter). Alternately, the necessary transcriptional and translational signals may also be supplied by the native promoter for the genes and/or their flanking regions.

The term “promoter” as used herein refers to a region of DNA that functions to control the transcription of one or more nucleic acid sequences as defined above, and that is structurally identified by the presence of a binding site for DNA-dependent RNA-polymerase and of other DNA sequences, which interact to regulate promoter function. A functional expression promoting fragment of a promoter is a shortened or truncated promoter sequence retaining the activity as a promoter. Promoter activity may be measured by any assay known in the art (see e.g. Wood, de Wet, Dewji, and DeLuca, (1984), Biochem Biophys. Res. Commun. 124, 592-596; Seliger and McElroy, (1960), Arch. Biochem. Biophys. 88, 136-141) or commercially available from Promega®).

An “enhancer region” to be used in the expression vector as defined herein, typically refers to a region of DNA that functions to increase the transcription of one or more genes. More specifically, the term “enhancer”, as used herein, is a DNA regulatory element that enhances, augments, improves, or ameliorates expression of a gene irrespective of its location and orientation vis-á-vis the gene to be expressed, and may be enhancing, augmenting, improving, or ameliorating expression of more than one promoter.

The promoter/enhancer sequences to be used in the expression vector as defined herein, may utilize plant, animal, insect, or fungus regulatory sequences. For example, promoter/enhancer elements can be used from yeast and other fungi (e.g. the GAL4 promoter, the alcohol dehydrogenase promoter, the phosphoglycerol kinase promoter, the alkaline phosphatase promoter). Alternatively, or in addition, they may include animal transcriptional control regions, e.g. (i) the insulin gene control region active within pancreatic beta-cells (see e.g. Hanahan, et al., 1985. Nature 315: 115-122); (ii) the immunoglobulin gene control region active within lymphoid cells (see e.g. Grosschedl, et al., 1984, Cell 38: 647-658); (iii) the albumin gene control region active within liver (see e.g. Pinckert, et al., 1987. Genes and Dev 1: 268-276; (iv) the myelin basic protein gene control region active within brain oligodendrocyte cells (see e.g. Readhead, et al., 1987, Cell 48: 703-712); and (v) the gonadotropin-releasing hormone gene control region active within the hypothalamus (see e.g. Mason, et al., 1986, Science 234: 1372-1378), and the like.

Additionally, the expression vector as defined herein may comprise an amplification marker. This amplification marker may be selected from the group consisting of, e.g. adenosine deaminase (ADA), dihydrofolate reductase (DHFR), multiple drug resistance gene (MDR), ornithine decarboxylase (ODC) and N-(phosphonacetyl)-L-aspartate resistance (CAD).

Exemplary expression vectors or their derivatives suitable for the present invention particularly include, e.g. human or animal viruses (e.g. vaccinia virus or adenovirus); insect viruses (e.g. baculovirus); yeast vectors; bacteriophage vectors (e.g. lambda phage); plasmid vectors and cosmid vectors.

The present invention additionally may utilize a variety of host-vector systems, which are capable of expressing the peptide coding sequence(s) of nucleic acids as defined above. These include, but are not limited to: (i) mammalian cell systems that are infected with vaccinia virus, adenovirus, and the like; (ii) insect cell systems infected with baculovirus and the like; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

Preferably, a host cell strain, suitable for such a host-vector system, may be selected that modulates the expression of inserted sequences of interest, or modifies or processes expressed peptides encoded by the sequences in the specific manner desired. In addition, expression from certain promoters may be enhanced in the presence of certain inducers in a selected host strain; thus facilitating control of the expression of a genetically-engineered peptide. Moreover, different host cells possess characteristic and specific mechanisms for the translational and post-translational processing and modification (e.g. glycosylation, phosphorylation, and the like) of expressed peptides. Appropriate cell lines or host systems may thus be chosen to ensure the desired modification and processing of the foreign peptide is achieved. For example, peptide expression within a bacterial system can be used to produce an non-glycosylated core peptide; whereas expression within mammalian cells ensures “native” glycosylation of a heterologous peptide.

Accordingly, the present invention also provides a cell comprising the vector as described above for use in (the preparation of a medicament for) preventing and/or treating Mild Cognitive Impairment, in particular Mild Cognitive Impairment due to Alzheimer's Disease.

The present invention further provides the use of antibodies directed against the JNK inhibitor sequences and/or chimeric peptides as described above, for preparing a pharmaceutical composition for the prevention and/or treatment of Mild Cognitive Impairment, in particular Mild Cognitive Impairment due to Alzheimer's Disease, as defined herein. In other words, the present invention also provides an antibody which binds immunospecifically to a JNK inhibitor sequence as defined in any of claims 1 to 9 or to a chimeric peptide as defined in any of claims 10 to 20 for use in preventing and/or treating Mild Cognitive Impairment, in particular Mild Cognitive Impairment due to Alzheimer's Disease. Furthermore, efficient means for production of antibodies specific for JNK inhibitor sequences according to the present invention, or for chimeric peptides containing such an inhibitor sequence, are described and may be utilized for this purpose.

According to the invention, JNK inhibitor sequences and/or chimeric peptides as defined herein, as well as, fragments, variants or derivatives thereof, may be utilized as immunogens to generate antibodies that immunospecifically bind these peptide components. Such antibodies include, e.g. polyclonal, monoclonal, chimeric, single chain, Fab fragments and a Fab expression library. In a specific embodiment the present invention provides antibodies to chimeric peptides or to JNK inhibitor sequences as defined above. Various procedures known within the art may be used for the production of these antibodies.

By way of example, various host animals may be immunized for production of polyclonal antibodies by injection with any chimeric peptide or JNK inhibitor sequence as defined above. Various adjuvants may be used thereby to increase the immunological response which include, but are not limited to, Freund's (complete and incomplete) adjuvant, mineral gels (e.g. aluminum hydroxide), surface active substances (e.g. lysolecithin, pluronic polyols, polyanions, peptides, oil emulsions, dinitrophenol, etc.), CpG, polymers, Pluronics, and human adjuvants such as Bacille Calmette-Guerin and Corynebacterium parvum.

For preparation of monoclonal antibodies directed towards an chimeric peptide or a JNK inhibitor sequence as defined above, any technique may be utilized that provides for the production of antibody molecules by continuous cell line culture. Such techniques include, but are not limited to, the hybridoma technique (see Kohler and Milstein, 1975. Nature 256: 495-497); the trioma technique; the human B-cell hybridoma technique (see Kozbor, et al., 1983, Immunol Today 4: 72) and the EBV hybridoma technique to produce human monoclonal antibodies (see Cole, et al., 1985. In: Monoclonal Antibodies and Cancer Therapy, Alan R. Liss, Inc., pp. 77-96). Human monoclonal antibodies may be utilized in the practice of the present invention and may be produced by the use of human hybridomas (see Cote, et al., 1983. Proc Natl Acad Sci USA 80: 2026-2030) or by transforming human B-cells with Epstein Barr Virus in vitro (see Cole, et al., 1985. In: Monoclonal Antibodies and Cancer Therapy (Alan R. Liss, Inc., pp. 77-96).

According to the invention, techniques can be adapted for the production of single-chain antibodies specific to the JNK inhibitor sequences and/or chimeric peptides (see e.g. U.S. Pat. No. 4,946,778) as defined herein. In addition, methods can be adapted for the construction of Fab expression libraries (see e.g. Huse et al., 1989. Science 246: 1275-1281) to allow rapid and effective identification of monoclonal Fab fragments with the desired specificity for these JNK inhibitor sequences and/or chimeric peptides. Non-human antibodies can be “humanized” by techniques well known in the art (see e.g. U.S. Pat. No. 5,225,539). Antibody fragments that contain the idiotypes to a JNK inhibitor sequences and/or chimeric peptide as defined herein may be produced by techniques known in the art including, e.g. (i) a F(ab′)₂ fragment produced by pepsin digestion of an antibody molecule; (ii) a Fab fragment generated by reducing the disulfide bridges of an F(ab′)₂ fragment; (iii) a Fab fragment generated by the treatment of the antibody molecule with papain and a reducing agent and (iv) Fv fragments.

In one embodiment of this invention, methods, that may be utilized for the screening of antibodies and which possess the desired specificity include, but are not limited to, enzyme-linked immunosorbent assay (ELISA) and other immunologically-mediated techniques known within the art. In a specific embodiment, selection of antibodies that are specific to a particular epitope of an JNK inhibitor sequence and/or an chimeric peptide as defined herein (e.g. a fragment thereof typically comprising a length of from 5 to 20, preferably 8 to 18 and most preferably 8 to 11 amino acids) is facilitated by generation of hybridomas that bind to the fragment of an JNK inhibitor sequence and/or an chimeric peptide, as defined herein, possessing such an epitope. These antibodies that are specific for an epitope as defined above are also provided herein.

The antibodies as defined herein may be used in methods known within the art referring to the localization and/or quantification of an JNK inhibitor sequence (and/or correspondingly to a chimeric peptide as defined above), e.g. for use in measuring levels of the peptide within appropriate physiological samples, for use in diagnostic methods, or for use in imaging the peptide, and the like.

The JNK inhibitor sequences, chimeric peptides, nucleic acids, vectors, host cells and/or antibodies as defined according to the invention can be formulated in a pharmaceutical composition, which may be applied in the prevention and/or treatment of Mild Cognitive Impairment, in particular Mild Cognitive Impairment due to Alzheimer's Disease, as defined herein. Accordingly, the present invention also provides a pharmaceutical composition comprising
(i) the JNK inhibitor sequence as described herein, the chimeric peptide as described herein, the nucleic acid as described herein, the vector as described herein, the (host) cell as described herein and/or the antibody as described herein; and
(ii) a pharmaceutically acceptable carrier
   for use in preventing and/or treating Mild Cognitive Impairment, in particular Mild Cognitive Impairment due to Alzheimer's Disease.

Typically, such a pharmaceutical composition used according to the present invention includes as an active component, e.g.: (i) any one or more of the JNK inhibitor sequences and/or chimeric peptides as defined above, and/or variants, fragments or derivatives thereof, particularly JNK inhibitor sequences according to any of sequences of SEQ ID NOs: 1 to 4 and 13 to 20 and 33-100 and/or chimeric peptides according to any of sequences of SEQ ID NOs: 9 to 12 and 23 to 32, preferably the chimeric peptide according to SEQ ID NO: 11, and/or JNK inhibitor sequences according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions; and/or (ii) nucleic acids encoding an JNK inhibitor sequence and/or an chimeric peptide as defined above and/or variants or fragments thereof, and/or (iii) cells comprising any one or more of the JNK inhibitor sequences and/or chimeric peptides, and/or variants, fragments or derivatives thereof, as defined above and/or (iv) cells transfected with a vector and/or nucleic acids encoding an JNK inhibitor sequence and/or an chimeric peptide as defined above and/or variants or fragments thereof.

According to a preferred embodiment, such a pharmaceutical composition as used according to the present invention typically comprises a safe and effective amount of a component as defined above, preferably of at least one JNK inhibitor sequence according to any of sequences of SEQ ID NOs: 1 to 4 and 13 to 20 and 33-100 and/or at least one chimeric peptide according to any of sequences of SEQ ID NOs: 9 to 12 and 23 to 32, preferably the chimeric peptide according to SEQ ID NO: 11, and/or at least one JNK inhibitor sequence according to any of sequences of SEQ ID NOs: 1 to 4 and 13 to 20 and 33-100 comprising a trafficking sequence according to any of SEQ ID NOs: 5-8 and 21 to 22, or variants or fragments thereof within the above definitions, or at least one nucleic acids encoding same, or at least one vector, host cell or antibody as defined above. It is particularly preferred that a pharmaceutical composition as used according to the present invention comprises as an active component a chimeric peptide comprising or consisting of the sequence according to SEQ ID NO: 11 or a functional sequence variant thereof as defined herein.

In addition, the pharmaceutical composition as used according to the present invention may additionally—i.e. in addition to any one or more of the JNK inhibitor sequences and/or chimeric peptides as defined above, and/or variants, fragments or derivatives thereof—also comprise optionally a further “active component”, which is also useful in Mild Cognitive Impairment, in particular in Mild Cognitive Impairment due to Alzheimer's Disease. In this context, the pharmaceutical composition according to the present invention may also combined in the therapy of Mild Cognitive Impairment, in particular Mild Cognitive Impairment due to Alzheimer's Disease, with a further pharmaceutical composition comprising a further “active component”. For example, a pharmaceutical composition comprising a JNK inhibitor and/or chimeric peptide according to the present invention used in the prevention and/or treatment of Mild Cognitive Impairment, in particular MCI due to Alzheimer's disease, as stand-alone therapy or in combination with PKR inhibitors and, optionally, in addition to the JNK inhibitor according to the present invention and the PKR inhibitor with a amyloid lowering agent.

Accordingly, it is preferred that the pharmaceutical composition further comprises a PKR inhibitor. Moreover, the pharmaceutical composition may further comprise an amyloid lowering agent and/or a glucocorticoid. Preferred PKR inhibitors, amyloid lowering agents and glucocorticoids as described above, in the context of the combination therapy, are also preferred in a pharmaceutical composition as described herein.

In the case of a combination therapy as described above, separate pharmaceutical compositions for the active components to be combined are preferred for better individual dosing, however for convenience also a single pharmaceutical composition comprising the active components to be combined is conceivable.

The inventors of the present invention additionally found, that the JNK-inhibitor sequence and the chimeric peptide, respectively, as defined herein, exhibit a particular well uptake rate into cells involved in Mild Cognitive Impairment, in particular MCI due to Alzheimer's disease. Therefore, the amount of a JNK-inhibitor sequence and chimeric peptide, respectively, in the pharmaceutical composition to be administered to a subject, may —without being limited thereto—have a very low dose. Thus, the dose may be much lower than for peptide drugs known in the art, such as DTS-108 (Florence Meyer-Losic et al., Clin Cancer Res., 2008, 2145-53). This has several positive aspects, for example a reduction of potential side reactions and a reduction in costs.

Preferably, in the JNK inhibitor sequence as described herein, in the chimeric peptide as described herein, in the combination therapy as described herein and in the pharmaceutical composition as described herein, the dose (per kg bodyweight) of the JNK inhibitor sequence as described herein or of the chimeric peptide as described herein is in the range of up to 10 mmol/kg, preferably up to 1 mmol/kg, more preferably up to 100 μmol/kg, even more preferably up to 10 μmol/kg, even more preferably up to 1 μmol/kg, even more preferably up to 100 nmol/kg, most preferably up to 50 nmol/kg.

It is also preferred that in the JNK inhibitor sequence as described herein, in the chimeric peptide as described herein, in the combination therapy as described herein and in the pharmaceutical composition as described herein, the dose (per kg bodyweight) of the JNK inhibitor sequence as described herein or of the chimeric peptide as described herein is in the range of up to 100 mg/kg, preferably up to 50 mg/kg, more preferably up to 10 mg/kg, and most preferably up to 1 mg/kg.

Thus, the dose range of the JNK inhibitor sequence as described herein or of the chimeric peptide as described herein may preferably be from about 0.01 pmol/kg to about 1 mmol/kg, from about 0.1 pmol/kg to about 0.1 mmol/kg, from about 1.0 pmol/kg to about 0.01 mmol/kg, from about 10 pmol/kg to about 1 μmol/kg, from about 50 pmol/kg to about 500 nmol/kg, from about 100 pmol/kg to about 300 nmol/kg, from about 200 pmol/kg to about 100 nmol/kg, from about 300 pmol/kg to about 50 nmol/kg, from about 500 pmol/kg to about 30 nmol/kg, from about 250 pmol/kg to about 5 nmol/kg, from about 750 pmol/kg to about 10 nmol/kg, from about 1 nmol/kg to about 50 nmol/kg, or a combination of any two of said values.

Preferably, in the JNK inhibitor sequence as described herein, in the chimeric peptide as described herein, in the combination therapy as described herein and in the pharmaceutical composition as described herein, the dose (per kg bodyweight) of the JNK inhibitor sequence as described herein or of the chimeric peptide as described herein is in the range of 1 μg/kg to 100 mg/kg, preferably 10 μg/kg to 50 mg/kg, more preferably 100 μg/kg to 10 mg/kg, and most preferably 500 μg/kg to 1 mg/kg.

In this context, prescription of treatment, e.g. decisions on dosage etc. when using the above pharmaceutical composition is typically within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in REMINGTON'S PHARMACEUTICAL SCIENCES, 16th edition, Osol, A. (ed), 1980. Accordingly, a “safe and effective amount” as defined above for components of the pharmaceutical compositions as used according to the present invention means an amount of each or all of these components, that is sufficient to significantly induce a positive modification of Mild Cognitive Impairment, in particular Mild Cognitive Impairment due to Alzheimer's Disease, as defined herein. At the same time, however, a “safe and effective amount” is small enough to avoid serious side-effects, that is to say to permit a sensible relationship between advantage and risk. The determination of these limits typically lies within the scope of sensible medical judgment. A “safe and effective amount” of such a component will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The pharmaceutical compositions according to the invention can be used according to the invention for human and also for veterinary medical purposes.

The pharmaceutical composition as used according to the present invention may furthermore comprise, in addition to one of these substances, a (compatible) pharmaceutically acceptable carrier, excipient, buffer, stabilizer or other materials well known to those skilled in the art.

In this context, the expression “(compatible) pharmaceutically acceptable carrier” preferably includes the liquid or non-liquid basis of the composition. The term “compatible” means that the constituents of the pharmaceutical composition as used herein are capable of being mixed with the pharmaceutically active component as defined above and with one another component in such a manner that no interaction occurs which would substantially reduce the pharmaceutical effectiveness of the composition under usual use conditions. Pharmaceutically acceptable carriers must, of course, have sufficiently high purity and sufficiently low toxicity to make them suitable for administration to a person to be treated.

If the pharmaceutical composition as used herein is provided in liquid form, the pharmaceutically acceptable carrier will typically comprise one or more (compatible) pharmaceutically acceptable liquid carriers. The composition may comprise as (compatible) pharmaceutically acceptable liquid carriers e.g. pyrogen-free water; isotonic saline, i.e. a solution of 0.9% NaCl, or buffered (aqueous) solutions, e.g. phosphate, citrate etc. buffered solutions, vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid, etc. Particularly for injection and/or infusion of the pharmaceutical composition as used herein, a buffer, preferably an aqueous buffer, and/or 0.9% NaCl may be used.

If the pharmaceutical composition as used herein is provided in solid form, the pharmaceutically acceptable carrier will typically comprise one or more (compatible) pharmaceutically acceptable solid carriers. The composition may comprise as (compatible) pharmaceutically acceptable solid carriers e.g. one or more compatible solid or liquid fillers or diluents or encapsulating compounds may be used as well, which are suitable for administration to a person. Some examples of such (compatible) pharmaceutically acceptable solid carriers are e.g. sugars, such as, for example, lactose, glucose and sucrose; starches, such as, for example, corn starch or potato starch; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulphate, etc.

The precise nature of the (compatible) pharmaceutically acceptable carrier or other material may depend on the route of administration. The choice of a (compatible) pharmaceutically acceptable carrier may thus be determined in principle by the manner in which the pharmaceutical composition as used according to the invention is administered. Various possible routes of administration are listed in the list “Route of Administration” of the FDA (cf. FDA: Data Standards Manual—Drug Nomenclature Monographs—Monograph Number: C-DRG-00301; Version Number 004), which is incorporated by reference herein. Further guidance for selecting an appropriate route of administration, in particular for non-human animals, can be found in Turner P V et al. (2011) Journal of the American Association for Laboratory Animal Science, Vol. 50, No 5, p. 600-613, which is also incorporated by reference herein. Preferred examples for routes for administration include parenteral routes (e.g. via injection), such as intravenous, intramuscular, subcutaneous, intradermal, or transdermal routes, etc., enteral routes, such as oral, or rectal routes, etc., topical routes, such as nasal, or intranasal routes, etc., or other routes, such as epidermal routes or patch delivery. More specifically, preferred routes of administration include (i) parenteral routes, including intravenous, intramuscular, subcutaneous, intradermal, transdermal; (ii) enteral routes, including orally, rectally; (iii) topical routes, including nasal, intranasal; (iv) administration routes avoiding the blood brain barrier, including intra-CSF, intrathecal; and (v) other routes, including epidermal or patch delivery.

The pharmaceutical composition as used according to the invention is preferably administered systemically. In general, routes for systemic administration include, for example, parenteral routes (e.g. via injection and/or infusion), such as intravenous, intra-arterial, intraosseous, intramuscular, subcutaneous, intradermal, transdermal, or transmucosal routes, etc., and enteral routes (e.g. as tablets, capsules, suppositories, via feeding tubes, gastrostomy), such as oral, gastrointestinal or rectal routes, etc. By systemic administration a system-wide action can be achieved and systemic administration is often very convenient, however, depending on the circumstances it may also trigger unwanted “side-effects” and/or higher concentrations of the JNK inhibitor according to the invention may be necessary as compared to local administration. Systemic administration is in general applicable for the prevention and/or treatment of Mild Cognitive impairment, in particular Mild Cognitive Impairment due to Alzheimer's Disease, due to its system-wide action. Preferred routes of systemic administration are intravenous, intramuscular, subcutaneous, oral and rectal administration, whereby intravenous and oral administration are particularly preferred.

For example, the oral route is usually the most convenient for a patient and carries the lowest cost. Therefore, oral administration is preferred for convenient systemic administration, if applicable. Pharmaceutical compositions for oral administration may be in tablet, capsule, powder or liquid form. A tablet may include a solid carrier as defined above, such as gelatin, and optionally an adjuvant. Liquid pharmaceutical compositions for oral administration generally may include a liquid carrier as defined above, such as water, petroleum, animal or vegetable oils, mineral oil or synthetic oil. Physiological saline solution, dextrose or other saccharide solution or glycols such as ethylene glycol, propylene glycol or polyethylene glycol may be included.

The pharmaceutical composition as used according to the invention can also be administered, for example, locally, for example intra-CSF, intrathecally. Thus, the JNK inhibitor or the chimeric peptide as described herein may be administered directly to the central nervous system (CNS). Such routes of administration include in particular epidural (peridural), intra-CSF (intra-cerebrospinal fluid), intracerebroventricular (intraventricular), intrathecal and intracerebral administration, for example administration into specific brain regions, whereby problems relating to the crossing of the blood-brain-barrier can be avoided.

Topical administration typically refers to application to body surfaces such as the skin or mucous membranes, whereas the more general term “local administration” additionally comprises application in and/or into specific parts of the body. Routes for local administration also include, for example, inhalational routes, such as nasal, or intranasal routes, administration through the mucous membranes in the body, etc., or other routes, such as epidermal routes, epicutaneous routes (application to the skin) or patch delivery and other local application, e.g. injection and/or infusion, into the organ or tissue to be treated etc. In local administration side effects are typically largely avoided. It is of note, that certain routes of administration may provide both, a local and a systemic effect, for example inhalation.

In general, the method of administration depends on various factors as mentioned above, for example the selected pharmaceutical carrier and the nature of the pharmaceutical preparation (e.g. as a liquid, tablet etc.) as well as the route of administration. For example, the pharmaceutical composition comprising the JNK inhibitor according to the invention may be prepared as a liquid, for example as a solution of the JNK inhibitor or the chimeric peptide according to the invention, preferably of the chimeric peptide according to a sequence of SEQ ID NO. 11, in 0.9% NaCl. A liquid pharmaceutical composition can be administered by various methods, for example as a spray (e.g., for inhalational, intranasal etc. routes), as a fluid for topical application, by injection, including bolus injection, by infusion, for example by using a pump, by instillation, but also p.o., e.g. as drops or drinking solution, in a patch delivery system etc. Accordingly, for the administration different devices may be used, in particular for injection and/or infusion, e.g. a syringe (including a pre-filled syringe); an injection device (e.g. the INJECT-EASET™ and GENJECTT™ device); an infusion pump (such as e.g. Accu-Chek™); an injector pen (such as the GENPENT™); a needleless device (e.g. MEDDECTOR™ and BIOJECTOR™); or an autoinjector.

The suitable amount of the pharmaceutical composition to be used can be determined by routine experiments with animal models. Such models include, without implying any limitation, for example rabbit, sheep, mouse, rat, gerbil, dog, pig and non-human primate models. Preferred unit dose forms for administration, in particular for injection and/or infusion, include sterile solutions of water, physiological saline or mixtures thereof. Usually, the pH of such solutions should be adjusted to about 7.4. Suitable carriers for administration, in particular for injection and/or infusion, include hydrogels, devices for controlled or delayed release, polylactic acid and collagen matrices. Suitable pharmaceutically acceptable carriers for topical application include those, which are suitable for use in lotions, creams, gels and the like. If the compound is to be administered perorally, tablets, capsules and the like are the preferred unit dose form. The pharmaceutically acceptable carriers for the preparation of unit dose forms, which can be used for oral administration are well known in the prior art. The choice thereof will depend on secondary considerations such as taste, costs and storability, which are not critical for the purposes of the present invention, and can be made without difficulty by a person skilled in the art.

For intravenous, intramuscular, intraperitoneal, cutaneous or subcutaneous injection and/or infusion, or injection and/or infusion at the site of affliction, i.e. local injection/infusion, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, in particular 0.9% NaCl, Ringer's Injection, Lactated Ringer's Injection. Preservatives, stabilizers, buffers, antioxidants and/or other additives may be included, as required. Whether it is a polypeptide, peptide, or nucleic acid molecule, other pharmaceutically useful compound according to the present invention that is to be given to an individual, administration is preferably in a “prophylactically effective amount or a “therapeutically effective amount” (as the case may be), this being sufficient to show benefit to the individual. The actual amount administered, and rate and time-course of administration, will depend on the nature and severity of what is being treated. For example, for i.v. administration in humans, single doses of the JNK inhibitor sequence or of the chimeric peptide of up to 10 mg per kg body weight are preferred, more preferably up to 1 mg per kg body weight, even more preferably up to 500 μg per kg body weight, for example in the range of 100 ng to 1 mg per kg body weight, more specifically in the range of 1 μg to 500 μg per kg body weight, even more specifically in the range of 5 μg to 100 μg per kg body weight. Such doses may be administered for example as injection and/or infusion, in particular as infusion, whereby the duration of the infusion varies for example between 1 to 90 min, preferably 10 to 70 min, more preferably 30 to 60 min.

Preferably, in the JNK inhibitor sequence as described herein, the chimeric peptide as described herein (in particular the chimeric peptide comprising SEQ ID NO: 11 as described herein), the combination as described herein, or the pharmaceutical composition as described herein, the JNK inhibitor sequence and/or the chimeric peptide is administered repeatedly, such as at least once per month, at least once per week, or at least once per day. Most preferably, the JNK inhibitor sequence and/or the chimeric peptide (in particular the chimeric peptide comprising SEQ ID NO: 11 as described herein) is administered repeatedly once per month or once every three weeks. In case of repeated administration, the above specified preferred dose ranges refer to single doses, which may be repeatedly administered.

Particularly preferably, the JNK inhibitor sequence or the chimeric peptide according to the present invention, for example the chimeric peptide having a sequence according to SEQ ID NO. 11, in particular in a pharmaceutical composition as defined herein, is applied in doses (per kg body weight) in the range of 1 μg/kg to 100 mg/kg, more preferably 10 μg/kg to 50 mg/kg, even more preferably 100 μg/kg to 10 mg/kg, and particularly preferably 500 μg/kg to 1 mg/kg. Thereby the JNK inhibitor sequence or the chimeric peptide is preferably administered, if applicable, once or repeatedly, such as daily for several, e.g. 2, 3, 4, 5, 6, 7, 8, 9, or 10, or more days, weeks, months or years. Preferably, the JNK inhibitor sequence and/or the chimeric peptide is administered weekly (once per week) for several, e.g. 2, 3, 4, 5, 6, 7, 8, 9, or 10, or more weeks, months or years; every second week (once per two weeks) for several, e.g. 2, 3, 4, 5, 6, 7, 8, 9, or 10, or more weeks, months or years; every third week (once per three weeks) for several, e.g. 2, 3, 4, 5, 6, 7, 8, 9, or 10, or more weeks, months or years; monthly (once per month) for several, e.g. 2, 3, 4, 5, 6, 7, 8, 9, or 10, or more months or years, every sixth week (once per every six weeks) for several, e.g. 2, 3, 4, 5, 6, 7, 8, 9, or 10, or more months or years, every second month (once per two months) for several, e.g. 2, 3, 4, 5, 6, 7, 8, 9, or 10, or more months or years; or every third month (once per three months) for several, e.g. 2, 3, 4, 5, 6, 7, 8, 9, or 10, or more weeks, months or years; more preferably weekly (once per week) for several, e.g. 2, 3, 4, 5, 6, 7, 8, 9, or 10, or more weeks, months or years; every second week (once per two weeks) for several, e.g. 2, 3, 4, 5, 6, 7, 8, 9, or 10, or more weeks, months or years; every third week (once per three weeks) for several, e.g. 2, 3, 4, 5, 6, 7, 8, 9, or 10, or more weeks, months or years; or monthly (once per month) for several, e.g. 2, 3, 4, 5, 6, 7, 8, 9, or 10, or more months or years; even more preferably every third week (once per three weeks) for several, e.g. 2, 3, 4, 5, 6, 7, 8, 9, or 10, or more weeks, months or years; or monthly (once per month) for several, e.g. 2, 3, 4, 5, 6, 7, 8, 9, or 10, or more months or years. Thereby, the JNK inhibitor sequence or the chimeric peptide is preferably applied systemically, e.g. i.v., p.o., i.m., or s.c., or intra-CSF (intra-cerebrospinal fluid). More preferably, the JNK inhibitor sequence or the chimeric peptide is administered i.v. or p.o.

Prevention and/or treatment of Mild Cognitive Impairment, in particular Mild Cognitive Impairment due to Alzheimer's Disease, typically includes administration of a pharmaceutical composition as defined above. The term “modulate” includes the suppression of expression of JNK when it is over-expressed in MCI. It also includes suppression of phosphorylation of c-jun, ATF2 or NFAT4 in MCI, for example, by using at least one JNK inhibitor sequence according to any of sequences of SEQ ID NOs: 1 to 4 and 13 to 20 and 33-100 and/or at least one chimeric peptide according to any of sequences of SEQ ID NOs: 9 to 12 and 23 to 32, whereby SEQ ID NO: 11 is particularly preferred, and/or at least one JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions, as a competitive inhibitor of the natural c-jun, ATF2 and NFAT4 binding site in a cell. The term “modulate” also includes suppression of hetero- and homomeric complexes of transcription factors made up of, without being limited thereto, c-jun, ATF2, or NFAT4 and their related partners, such as for example the Aβ-1 complex that is made up of c-jun, AFT2 and c-fos. When Mild Cognitive Impairment, in particular Mild Cognitive Impairment due to Alzheimer's Disease, as defined above is associated with JNK overexpression, such suppressive JNK inhibitor sequences can be introduced to a cell. In some instances, “modulate” may then include the increase of JNK expression, for example by use of an IB peptide-specific antibody that blocks the binding of an IB-peptide to JNK, thus preventing JNK inhibition by the IB-related peptide.

Prevention and/or treatment of a subject with the JNK inhibitor sequence, the chimeric peptide or the pharmaceutical composition as disclosed herein may be typically accomplished by administering (in vivo) an (“therapeutically effective”) amount of said pharmaceutical composition to a subject. The term “therapeutically effective” means that the active component of the pharmaceutical composition is of sufficient quantity to ameliorate Mild Cognitive Impairment, in particular Mild Cognitive Impairment due to Alzheimer's Disease, as defined above.

The subject to be treated with the JNK inhibitor sequence, the chimeric peptide or the pharmaceutical composition as disclosed herein may be e.g. any mammal, a human, a primate, mouse, rat, dog, cat, cow, horse or pig, whereby a human is particularly preferred.

Accordingly, any peptide as defined above, e.g. at least one JNK inhibitor sequence according to any of sequences of SEQ ID NOs: 1 to 4 and 13 to 20 and 33-100 and/or at least one chimeric peptide according to any of sequences of SEQ ID NOs: 9 to 12 and 23 to 32, preferably SEQ ID NO: 11, and/or at least one JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions, may be utilized in a specific embodiment of the present invention to treat Mild Cognitive Impairment, in particular Mild Cognitive Impairment due to Alzheimer's Disease, as defined above, e.g. by modulating activated JNK signaling pathways.

However, the above defined peptides may be also encoded by nucleic acids, which then may form part of the inventive pharmaceutical compositions, e.g. for use in gene therapy. In this context, gene therapy refers to therapy that is performed by administration of a specific nucleic acid as defined above to a subject, e.g. by way of a pharmaceutical composition as defined above, wherein the nucleic acid(s) exclusively comprise(s) L-amino acids. In this embodiment of the present invention, the nucleic acid produces its encoded peptide(s), which then serve(s) to exert a therapeutic effect by modulating function of MCI. Any of the methods relating to gene therapy available within the art may be used in the practice of the present invention (see e.g. Goldspiel, et al., 1993. Clin Pharm 12: 488-505).

In a preferred embodiment, the nucleic acid as defined above and as used for gene therapy is part of an expression vector encoding and expressing any one or more of the IB-related peptides as defined above within a suitable host, i.e. an JNK inhibitor sequence according to any of sequences of SEQ ID NOs: 1 to 4 and 13 to 20 and 33-100 and/or a chimeric peptide according to any of sequences of SEQ ID NOs: 9 to 12 and 23 to 32, and/or an JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions. In a specific embodiment, such an expression vector possesses a promoter that is operably-linked to coding region(s) of a JNK inhibitor sequence. The promoter may be defined as above, e.g. inducible or constitutive, and, optionally, tissue-specific.

In another specific embodiment, a nucleic acid molecule as defined above is used for gene therapy, in which the coding sequences of the nucleic acid molecule (and any other desired sequences thereof) as defined above are flanked by regions that promote homologous recombination at a desired site within the genome, thus providing for intra-chromosomal expression of these nucleic acids (see e.g. Koller and Smithies, 1989. Proc Natl Acad Sci USA 86: 8932-8935).

Delivery of the nucleic acid as defined above according to the invention into a patient for the purpose of gene therapy, particular in the context of the above mentioned Mild Cognitive Impairment, in particular Mild Cognitive Impairment due to Alzheimer's Disease, as defined above may be either direct (i.e. the patient is directly exposed to the nucleic acid or nucleic acid-containing vector) or indirect (i.e. cells are first transformed with the nucleic acid in vitro, then transplanted into the patient), whereby in general the routes of administration as mentioned above for the pharmaceutical composition apply as well, however, a local administration for example by local injection into the tissue or organ to be treated is preferred. These two approaches are known, respectively, as in vivo or ex vivo gene therapy. In a specific embodiment of the present invention, a nucleic acid is directly administered in vivo, where it is expressed to produce the encoded product. This may be accomplished by any of numerous methods known in the art including, e.g. constructing the nucleic acid as part of an appropriate nucleic acid expression vector and administering the same in a manner such that it becomes intracellular (e.g. by infection using a defective or attenuated retroviral, adeno-associated viral or other viral vector; see U.S. Pat. No. 4,980,286); directly injecting naked DNA; using microparticle bombardment (e.g. a “GeneGun”; Biolistic, DuPont); coating the nucleic acids with lipids; using associated cell-surface receptors/transfecting agents; encapsulating in liposomes, microparticles, or microcapsules; administering it in linkage to a peptide that is known to enter the nucleus; or by administering it in linkage to a ligand predisposed to receptor-mediated endocytosis (see e.g. Wu and Wu, 1987. J Biol Chem 262: 4429-4432), which can be used to “target” cell types that specifically express the receptors of interest, etc.

An additional approach to gene therapy in the practice of the present invention involves transferring a gene (comprising a nucleic acid as defined above) into cells in in vitro tissue culture by such methods as electroporation, lipofection, calcium phosphate-mediated transfection, viral infection, or the like. Generally, the method of transfer includes the concomitant transfer of a selectable marker to the cells. The cells are then placed under selection pressure (e.g. antibiotic resistance) so as to facilitate the isolation of those cells that have taken up, and are expressing, the transferred gene. Those cells are then delivered to a patient. In a specific embodiment, prior to the in vivo administration of the resulting recombinant cell, the nucleic acid is introduced into a cell by any method known within the art including e.g. transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methods that ensure that the necessary developmental and physiological functions of the recipient cells are not disrupted by the transfer. See e.g. Loeffler and Behr, 1993. Meth Enzymol 217: 599-618. The chosen technique should provide for the stable transfer of the nucleic acid to the cell, such that the nucleic acid is expressible by the cell. Preferably, the transferred nucleic acid is heritable and expressible by the cell progeny.

In preferred embodiments of the present invention, the resulting recombinant cells may be delivered to a patient by various methods known within the art including, e.g. injection of epithelial cells (e.g. subcutaneously), application of recombinant skin cells as a skin graft onto the patient, and intravenous injection of recombinant blood cells (e.g. hematopoietic stem or progenitor cells). The total amount of cells that are envisioned for use depend upon the desired effect, patient state, and the like, and may be determined by one skilled within the art. Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, keratinocytes, fibroblasts, muscle cells, hepatocytes and blood cells, or various stem or progenitor cells, in particular embryonic heart muscle cells, liver stem cells (International Patent Publication WO 94/08598), neural stem cells (Stemple and Anderson, 1992, Cell 71: 973-985), hematopoietic stem or progenitor cells, e.g. as obtained from bone marrow, umbilical cord blood, peripheral blood, fetal liver, and the like. In a preferred embodiment, the cells utilized for gene therapy are autologous to the patient.

Alternatively and/or additionally, for treating MCI as mentioned herein targeting therapies may be used to deliver the JNK inhibitor sequences, chimeric peptides, and/or nucleic acids as defined above more specifically to certain types of cell, by the use of targeting systems such as (a targeting) antibody or cell specific ligands. Antibodies used for targeting are typically specific for cell surface proteins of cells associated with MCI. By way of example, these antibodies may be directed to cell surface antibodies such as e.g. B cell-associated surface proteins such as MHC class II DR protein, CD18 (LFA-1 beta chain), CD45RO, CD40 or Bgp95, or cell surface proteins selected from e.g. CD2, CD4, CD5, CD7, CD8, CD9, CD10, CD13, CD16, CD19, CD20, CD21, CD22, CD23, CD24, CD25, CD30, CD33, CD34, CD38, CD39, CD4, CD43, CD45, CD52, CD56, CD68, CD71, CD138, etc. Targeting constructs may be typically prepared by covalently binding the JNK inhibitor sequences, chimeric peptides, and nucleic acids as defined herein according to the invention to an antibody specific for a cell surface protein or by binding to a cell specific ligand. Proteins may e.g. be bound to such an antibody or may be attached thereto by a peptide bond or by chemical coupling, crosslinking, etc. The targeting therapy may then be carried out by administering the targeting construct in a pharmaceutically efficient amount to a patient by any of the administration routes as defined below, e.g. intraperitoneal, nasal, intravenous, oral and patch delivery routes. Preferably, the JNK inhibitor sequences, chimeric peptides, or nucleic acids as defined herein according to the invention, being attached to the targeting antibodies or cell specific ligands as defined above, may be released in vitro or in vivo, e.g. by hydrolysis of the covalent bond, by peptidases or by any other suitable method. Alternatively, if the JNK inhibitor sequences, chimeric peptides, or nucleic acids as defined herein according to the invention are attached to a small cell specific ligand, release of the ligand may not be carried out. If present at the cell surface, the chimeric peptides may enter the cell upon the activity of its trafficking sequence. Targeting may be desirable for a variety of reasons; for example if the JNK inhibitor sequences, chimeric peptides, and nucleic acids as defined herein according to the invention are unacceptably toxic or if it would otherwise require a too high dosage. Instead of administering the JNK inhibitor sequences and/or chimeric peptides as defined herein according to the invention directly, they could be produced in the target cells by expression from an encoding gene introduced into the cells, e.g. from a viral vector to be administered. The viral vector typically encodes the JNK inhibitor sequences and/or chimeric peptides as defined herein according to the invention. The vector could be targeted to the specific cells to be treated. Moreover, the vector could contain regulatory elements, which are switched on more or less selectively by the target cells upon defined regulation. This technique represents a variant of the VDEPT technique (virus-directed enzyme prodrug therapy), which utilizes mature proteins instead of their precursor forms.

Alternatively, the JNK inhibitor sequences and/or chimeric peptides as defined herein could be administered in a precursor form by use of an antibody or a virus. These JNK inhibitor sequences and/or chimeric peptides may then be converted into the active form by an activating agent produced in, or targeted to, the cells to be treated. This type of approach is sometimes known as ADEPT (antibody-directed enzyme prodrug therapy) or VDEPT (virus-directed enzyme prodrug therapy); the former involving targeting the activating agent to the cells by conjugation to a cell-specific antibody, while the latter involves producing the activating agent, e.g. a JNK inhibitor sequence or the chimeric peptide, in a vector by expression from encoding DNA in a viral vector (see for example, EP-A-415731 and WO 90/07936).

According to a further embodiment, the JNK inhibitor sequences, chimeric peptides, nucleic acid sequences or antibodies to JNK inhibitor sequences or to chimeric peptides as defined herein, e.g. an JNK inhibitor sequence according to any of sequences of SEQ ID NOs: 1 to 4 and 13 to 20 and 33-100 and/or a chimeric peptide according to any of sequences of SEQ ID NOs: 9 to 12 and 23 to 32, and/or an JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions, may be utilized in (in vitro) assays (e.g. immunoassays) to detect, prognose, diagnose, or monitor Mild Cognitive Impairment, in particular Mild Cognitive Impairment due to Alzheimer's Disease, as defined above, or monitor the treatment thereof. The immunoassay may be performed by a method comprising contacting a sample derived from a patient with an antibody to an JNK inhibitor sequence, a chimeric peptide, or a nucleic acid sequence, as defined above, under conditions such that immunospecific-binding may occur, and subsequently detecting or measuring the amount of any immunospecific-binding by the antibody. In a specific embodiment, an antibody specific for an JNK inhibitor sequence, a chimeric peptide or a nucleic acid sequence may be used to analyze a tissue or serum sample from a patient for the presence of JNK or a JNK inhibitor sequence; wherein an aberrant level of JNK is indicative of a diseased condition. The immunoassays that may be utilized include, but are not limited to, competitive and non-competitive assay systems using techniques such as Western Blots, radioimmunoassays (RIA), enzyme linked immunosorbent assay (ELISA), “sandwich” immunoassays, immunoprecipitation assays, precipitin reactions, gel diffusion precipitin reactions, immunodiffusion assays, agglutination assays, fluorescent immunoassays, complement-fixation assays, immunoradiometric assays, and protein-A immunoassays, etc. Alternatively, (in vitro) assays may be performed by delivering the JNK inhibitor sequences, chimeric peptides, nucleic acid sequences or antibodies to JNK inhibitor sequences or to chimeric peptides, as defined above, to target cells typically selected from e.g. cultured animal cells, human cells or micro-organisms, and to monitor the cell response by biophysical methods typically known to a skilled person. The target cells typically used therein may be cultured cells (in vitro) or in vivo cells, i.e. cells composing the organs or tissues of living animals or humans, or microorganisms found in living animals or humans.

The present invention additionally provides the use of kits for diagnostic or therapeutic purposes, particular for the treatment, prevention or monitoring of Mild Cognitive Impairment, in particular Mild Cognitive Impairment due to Alzheimer's Disease, as defined above, wherein the kit includes one or more containers containing JNK inhibitor sequences, chimeric peptides, nucleic acid sequences and/or antibodies to these JNK inhibitor sequences or to chimeric peptides as defined above, e.g. an anti-JNK inhibitor sequence antibody to an JNK inhibitor sequence according to any of sequences of SEQ ID NOs: 1 to 4 and 13 to 20 and 33-100, to a chimeric peptide according to any of sequences of SEQ ID NOs: 9 to 12 and 23 to 32, to an JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or to or variants or fragments thereof within the above definitions, or such an anti-JNK inhibitor sequence antibody and, optionally, a labeled binding partner to the antibody. The label incorporated thereby into the antibody may include, but is not limited to, a chemiluminescent, enzymatic, fluorescent, colorimetric or radioactive moiety. In another specific embodiment, kits for diagnostic use in the treatment, prevention or monitoring of Mild Cognitive Impairment, in particular Mild Cognitive Impairment due to Alzheimer's Disease, as defined above are provided which comprise one or more containers containing nucleic acids that encode, or alternatively, that are the complement to, an JNK inhibitor sequence and/or a chimeric peptide as defined above, optionally, a labeled binding partner to these nucleic acids, are also provided. In an alternative specific embodiment, the kit may be used for the above purposes as a kit, comprising one or more containers, a pair of oligonucleotide primers (e.g. each 6-30 nucleotides in length) that are capable of acting as amplification primers for polymerase chain reaction (PCR; see e.g. Innis, et al., 1990. PCR PROTOCOLS, Academic Press, Inc., San Diego, Calif.), ligase chain reaction, cyclic probe reaction, and the like, or other methods known within the art used in context with the nucleic acids as defined above. The kit may, optionally, further comprise a predetermined amount of a purified JNK inhibitor sequence as defined above, a chimeric peptide as defined above, or nucleic acids encoding these, for use as a diagnostic, standard, or control in the assays for the above purposes.

In a further aspect, the present invention also provides a method of preventing and/or treating Mild Cognitive Impairment, in particular Mild Cognitive Impairment due to Alzheimer's Disease, in a subject in need thereof comprising administering to the subject the JNK inhibitor sequence as described herein or of the chimeric peptide as described herein.

In a preferred embodiment of such a method, the JNK inhibitor sequence as described herein, the chimeric peptide as described herein, the combination as described herein or the pharmaceutical composition as described herein is administered to the subject.

In another preferred embodiment of said method, the subject was diagnosed with Mild Cognitive Impairment, preferably with amnestic or non-amnestic Mild Cognitive Impairment, more preferably with amnestic Mild Cognitive Impairment, even more preferably with Mild Cognitive Impairment due to Alzheimer's Disease.

Further preferred embodiments of the method according to the present invention can be derived from preferred embodiments of the JNK inhibitor sequence as described herein, the chimeric peptide as described herein, the combination as described herein or the pharmaceutical composition as described as well as the preferred administration routes, doses and treatment schedules as described herein.

The present invention is not to be limited in scope by the specific embodiments described herein. Indeed, various modifications of the invention in addition to those described herein will become apparent to those skilled in the art from the foregoing description and accompanying figures. Such modifications fall within the scope of the appended claims.

Various publications are cited herein, the disclosures of which are incorporated by reference in their entirety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

### DESCRIPTION OF FIGURES

FIG. 1 are diagrams showing alignments of conserved JBD domain regions in the indicated transcription factors. JNK inhibitor sequences used herein were identified by carrying out sequence alignments. The results of this alignment are exemplarily shown in FIGS. 1A-1C. FIG. 1A depicts the region of highest homology between the JBDs of IB1, IB2, c-Jun and ATF2. Panel B depicts the amino acid sequence of the JBDs of L-IB1(s) and L-IB1 for comparative reasons. Fully conserved residues are indicated by asterisks, while residues changed to Ala in the GFP-JBD_{23Mut} vector are indicated by open circles. FIG. 1C shows the amino acid sequences of chimeric proteins that include a JNK inhibitor sequence and a trafficking sequence. In the example shown, the trafficking sequence is derived from the human immunodeficiency virus (HIV) TAT polypeptide, and the JNK inhibitor sequence is derived from an IB1(s) polypeptide. Human, mouse, and rat sequences are identical in Panels B and C.

FIG. 2 is a diagram showing sequences of generic TAT-IB fusion peptides from human, mouse and rat.

FIG. 3 depicts the results of the inhibition of endogeneous JNK-activity in HepG2 cells using fusion peptides according to SEQ ID NOs: 9 and 11 in an one-well approach. As can be seen from FIG. 3, particularly panel d in FIG. 3, D-TAT-IB1(s) according to SEQ ID NO: 11 (here abbreviated as D-JNKI) effectively inhibits JNK activity, even better than L-TAT-IB1(s) according to SEQ ID NO: 9 (here abbreviated as L-JNKI).

FIG. 4 shows for Example 12 the JNK activity in the cortex (A) and in the hippocampus (B) of wildtype (WT) and 5XFAD mice treated for 3 to 6 months with either (i) 10 mg/kg of the JNK peptide inhibitor XG-102 (SEQ ID NO: 11) or (ii) saline. Data are means±SEM (n≥6). *P<0.05, **P<0.01, and ***P<0.001.

FIG. 5 shows for Example 13 the cJun activity in the cortex (A) and hippocampus (B) of wildtype (WT) and 5XFAD mice treated for 3 to 6 months with either (i) 10 mg/kg of the JNK peptide inhibitor XG-102 (SEQ ID NO: 11) or (ii) saline. Data are means±SEM (n≥6). *P<0.05, and ***P<0.001.

FIG. 6 shows for Example 14 the effect of XG-102 (SEQ ID NO: 11) on Aβ42 clone monoclonal antibody (“MOAB”) in the Layer 5 cortex. (A) Sagittal brain sections from 5XFAD mice treated with saline or XG-102 for 3 months were incubated with an antibody against Aβ42, visualized by DAB staining, and micro photographed in Layer 5 cortex. (B) Quantification of Aβ42 labelling in Layer 5 cortex. n≥6; *P<0.05.

FIG. 7 shows for Example 15 the effect of XG-102 (SEQ ID NO: 11) on human pAPP levels in 5XFAD mice in the hippocampus. Histograms show the level of mixed mice and human pAPP of 5XFAD and wildtype (WT) mice treated with saline or XG-102 for 3 or 6 months. Data are means±SEM (n≥4). *P<0.05, and ***P<0.001.

FIG. 8 shows for Example 16 the effect of XG-102 (SEQ ID NO: 11) on the levels of cleaved caspase 3 in cortex (A) and hippocampus (B) in wildtype (WT) and 5XFAD mice treated for 3 to 6 months. Data are means±SEM (n≥3). *P<0.05, **P<0.01, and ***P<0.001.

FIG. 9 shows for Example 17 the effects of a treatment with XG-102 (SEQ ID NO: 11) on brain levels of phosphorylated Bcl2 on Serine 87 in cortex (A) and hippocampus (B) of wildtype (WT) and 5XFAD mice treated for 3 to 6 months. Data are means±SEM (n≥3). *P<0.05, **P<0.01, and ***P<0.001.

FIG. 10 shows for Example 18 the effects of a treatment with XG-102 (SEQ ID NO: 11) on caspase 3 activity in cortex (A) and hippocampus (B) of wildtype (WT) and 5XFAD mice treated for 3 to 6 months. Data are means±SEM (n≥6). **P<0.01, and ***P<0.001.

FIG. 11 shows for Example 19 the effects of a treatment with XG-102 (SEQ ID NO: 11) on cytokine IL-1 levels in the cortex of WT and 5XFAD mice treated for 3 to 6 months. Data are means±SEM (n≥6). ***P<0.001.

FIG. 12 shows for Example 20 the effects of XG-102 (SEQ ID NO: 11) on memory in wildtype (WT) and 5XFAD mice. Mice were tested for spatial and procedural working memory in the Y-maze task. During 8-min testing, the spontaneous alternation behavior between the three arms of the maze was measured. Data are means±SEM (n≥6). *P<0.05, **P<0.01, and ***P<0.001.

### EXAMPLES

### Example 1: Identification of INK Inhibitor Sequences

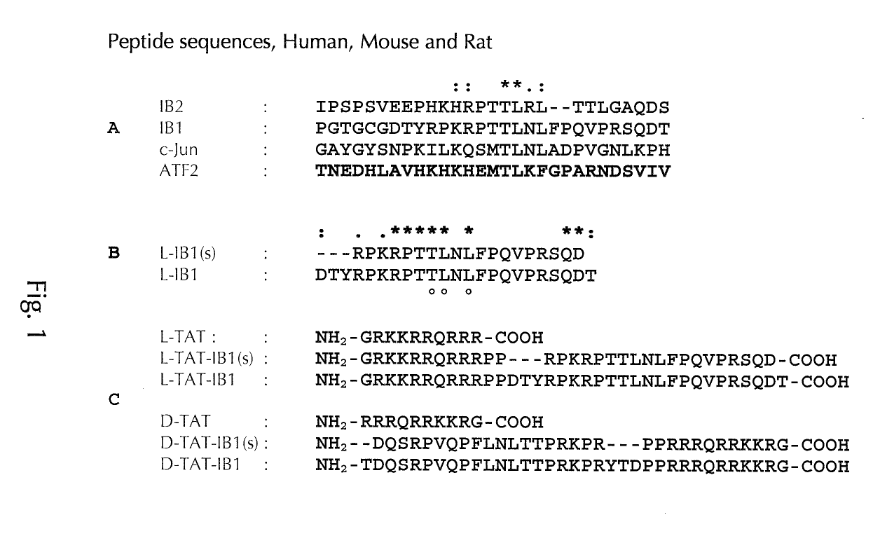

Amino acid sequences important for efficient interaction with JNK were identified by sequence alignments between known JNK binding domain JBDs. A sequence comparison between the JBDs of IB1 [SEQ ID NO: 13], IB2 [SEQ ID NO: 14], c-Jun [SEQ ID NO: 15] and ATF2 [SEQ ID NO: 16] defined a weakly conserved 8 amino acid sequence (see FIG. 1A). Since the JBDs of IB1 and IB2 are approximately 100 fold as efficient as c-Jun or ATF2 in binding JNK (Dickens et al. Science 277: 693 (1997), it was reasoned that conserved residues between IB1 and IB2 must be important to confer maximal binding. The comparison between the JBDs of IB1 and IB2 defined two blocks of seven and three amino acids that are highly conserved between the two sequences.

These two blocks are contained within a peptide sequence of 19 amino acids in L-IB1 (s) [SEQ ID NO: 1] and are also shown for comparative reasons in a 23 aa peptide sequence derived from IB1 [SEQ ID NO: 17]. These sequences are shown in FIG. 1B, dashes in the L-IB1 sequence indicate a gap in the sequence in order to align the conserved residues with L-IB1(s).

### Example 2: Preparation of INK Inhibitor Fusion Proteins

JNK inhibitor fusion proteins according to SEQ ID NO: 9 were synthesized by covalently linking the C-terminal end of SEQ ID NO: 1 to a N-terminal 10 amino acid long carrier peptide derived from the HIV-TAT4g 57 (Vives et al., J Biol. Chem. 272: 16010 (1997)) according to SEQ ID NO: 5 via a linker consisting of two proline residues. This linker was used to allow for maximal flexibility and prevent unwanted secondary structural changes. The basic constructs were also prepared and designated L-IB1 (s) (SEQ ID NO: 1) and L-TAT [SEQ ID NO: 5], respectively.

All D retro-inverso peptides according to SEQ ID NO: 11 were synthesized accordingly. The basic constructs were also prepared and designated D-IB1 (s) [SEQ ID NO: 2] and D-TAT [SEQ ID NO: 6], respectively.

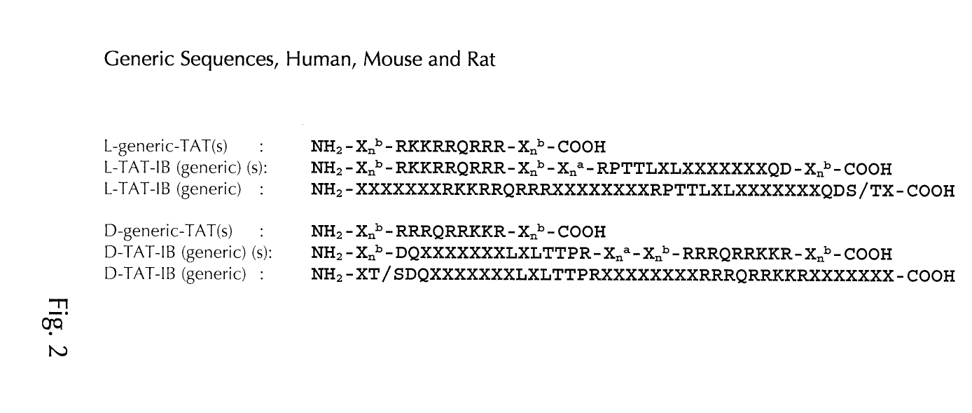

All D and L fusion peptides according to SEQ ID NOs: 9, 10, 11 and 12 were produced by classical Fmock synthesis and further analysed by Mass Spectrometry. They were finally purified by HPLC. To determine the effects of the proline linker, two types of TAT peptide were produced one with and one without two prolines. The addition of the two prolines did not appear to modify the entry or the localization of the TAT peptide inside cells. Generic peptides showing the conserved amino acid residues are given in FIG. 2.

### Example 3: Inhibition of Cell Death by JBD19

Effects of the 19 aa long JBD sequence of IB1 (s) on JNK biological activities were studied. The 19 aa sequence was linked N-terminal to the Green Fluorescent Protein (GFP JBD19 construct), and the effect of this construct on pancreatic beta-cell apoptosis induced by IL1 was evaluated. This mode of apoptosis was previously shown to be blocked by transfection with JBD₁₋₂₈₀ whereas specific inhibitors of ERK1/2 or p38 as known in the art did not protect.

Oligonucleotides corresponding to JBD19 and comprising a conserved sequence of 19 amino acids as well as a sequence mutated at the fully conserved regions were synthesized and directionally inserted into the EcoRI and SalI sites of the pEGFP-N1 vector encoding the Green Fluorescent Protein (GFP) (from Clontech). Insulin producing βTC-3 cells were cultured in RPMI 1640 medium supplemented with 10% Fetal Calf Serum, 100 μg/mL Streptomycin, 100 units/mL Penicillin and 2 mM Glutamine. Insulin producing βTC-3 cells were transfected with the indicated vectors and IL-1β (10 ng/mL) was added to the cell culture medium. The number of apoptotic cells was counted at 48 hours after the addition of IL-1β using an inverted fluorescence microscope. Apoptotic cells were discriminated from normal cells by the characteristic “blebbing out” of the cytoplasm and were counted after two days.

GFP is Green Fluorescent protein expression vector used as a control; JBD19 is the vector expressing a chimeric GFP linked to the 19 aa sequence derived from the JBD of IB1; JBD19Mut is the same vector as GFP-JBD19, but with a JBD mutated at four conserved residues shown as FIG. 1B; and JBD₁₋₂₈₀ is the GFP vector linked to the entire JBD (aa 1-280). The GFP-JBD19 expressing construct prevented IL-1β induced pancreatic β-cell apoptosis as efficiently as the entire JBD₁₋₂₈₀.

As additional controls, sequences mutated at fully conserved IB1(s) residues had greatly decreased ability to prevent apoptosis.

### Example 4: Cellular Import of TAT-IB1 (s) Peptides

The ability of the L- and D-enantiomeric forms of TAT and TAT-IB(s) peptides (“TAT-IB peptides”) to enter cells was evaluated. L-TAT, D-TAT, L-TAT-IB1(s), and D-TAT-IB1(s) peptides [SEQ ID NOs: 5, 6, 9 and 12, respectively] were labeled by N-terminal addition of a glycine residue conjugated to fluorescein. Labeled peptides (1 PM) were added to βTC-3 cell cultures, which were maintained as described in Example 3. At predetermined times cells were washed with PBS and fixed for five minutes in ice-cold methanol-acetone (1:1) before being examined under a fluorescence microscope. Fluorescein-labeled BSA (1 μM, 12 moles/mole BSA) was used as a control. Results demonstrated that all the above fluorescein labeled peptides had efficiently and rapidly (less than five minutes) entered cells once added to the culture medium. Conversely, fluorescein labeled bovine serum albumin (1 μM BSA, 12 moles fluorescein/mole BSA) did not enter the cells.

A time course study indicated that the intensity of the fluorescent signal for the L-enantiomeric peptides decreased by 70% following a 24 hours period. Little to no signal was present at 48 hours. In contrast, D-TAT and D-TAT-IB1(s) were extremely stable inside the cells.

Fluorescent signals from these all-D retro-inverso peptides were still very strong 1 week later, and the signal was only slightly diminished at 2 weeks post treatment.

### Example 5: In Vitro Inhibition of c-JUN, ATF2 and Elk1 Phosphorylation

The effects of the peptides on JNKs-mediated phosphorylation of their target transcription factors were investigated in vitro. Recombinant and non activated JNK1, JNK2 and JNK3 were produced using a TRANSCRIPTION AND TRANSLATION rabbit reticulocyte lysate kit (Promega) and used in solid phase kinase assays with c-Jun, ATF2 and Elk1, either alone or fused to glutathione-S-transferase (GST), as substrates. Dose response studies were performed wherein L-TAT or L-TAT-IB1(s) peptides (0-25 μM) were mixed with the recombinant JNK1, JNK2, or JNK3 kinases in reaction buffer (20 mM Tris-acetate, 1 mM EGTA, 10 mM p-nitrophenyl-phosphate (pNPP), 5 mM sodium pyrophosphate, 10 mM p-glycerophosphate, 1 mM dithiothreitol) for 20 minutes. The kinase reactions were then initiated by the addition of 10 mM MgCl₂ and 5 pCi ³³P-gamma-dATP and 1 μg of either GST-Jun (aa 1-89), GST-AFT2 (aa 1-96) or GST-ELK1 (aa 307-428). GST-fusion proteins were purchased from Stratagene (La Jolla, Calif.).

Ten μL of glutathione-agarose beads were also added to the mixture. Reaction products were then separated by SDS-PAGE on a denaturing 10% polyacrylamide gel. Gels were dried and subsequently exposed to X-ray films (Kodak). Nearly complete inhibition of c-Jun, ATF2 and Elk1 phosphorylation by JNKs was observed at TAT-IB(s) peptide doses as low as 2.5 [M. However, a marked exception was the absence of TAT-IB(s) inhibition of JNK3 phosphorylation of Elk]. Overall, the TAT-IB1(s) peptide showed superior effects in inhibiting JNK family phosphorylation of their target transcription factors. The ability of D-TAT, D-TAT-IB1(s) and L-TAT-IB1(s) peptides (0-250 μM dosage study) to inhibit GST-Jun (aa 1-73) phosphorylation by recombinant JNK1, JNK2, and JNK3 by were analyzed as described above. Overall, D-TAT-IB1 (s) peptide decreased JNK-mediated phosphorylation of c-Jun, but at levels approximately 10-20 fold less efficiently than L-TAT-IB1 (s).

### Example 6: Inhibition of c-JUN Phosphorylation by Activated JNKs

The effects of the L-TAT or L-TAT-IB1(s) peptides as defined herein on JNKs activated by stressful stimuli were evaluated using GST-Jun to pull down JNKs from UV-light irradiated HeLa cells or IL-1β treated PTC cells. PTC cells were cultured as described above. HeLa cells were cultured in DMEM medium supplemented with 10% Fetal Calf Serum, 100 μg/mL Streptomycin, 100 units/ml Penicillin and 2 mM Glutamine. One hour prior to being used for cell extract preparation, PTC cells were activated with IL-1β as described above, whereas HeLa cells were activated by UV-light (20 J/m²). Cell extracts were prepared from control, UV-light irradiated HeLa cells and IL-1β treated βTC-3 cells by scraping the cell cultures in lysis buffer (20 mM Tris-acetate, 1 mM EGTA, 1% Triton X-100, 10 mM p-nitrophenyl-phosphate, 5 mM sodium pyrophosphate, 10 mMP-glycerophosphate, 1 mM dithiothreitol). Debris was removed by centrifugation for five minutes at 15,000 rpm in an SS-34 Beckman rotor. One-hundred μg extracts were incubated for one hour at room temperature with one μg GST-jun (amino acids 1-89) and 10 μL of glutathione-agarose beads (Sigma). Following four washes with the scraping buffer, the beads were resuspended in the same buffer supplemented with L-TAT or L-TAT-IB1(s) peptides (25 μM) for 20 minutes. Kinase reactions were then initiated by addition of 10 mM MgCl₂ and 5 pCi ³³P-gamma-dATP and incubated for 30 minutes at 30° C.

Reaction products were then separated by SDS-PAGE on a denaturing 10% polyacrylamide gel. Gels were dried and subsequently exposed to X-ray films (Kodak). The TAT-IB(s) peptides efficiently prevented phosphorylation of c-Jun by activated JNKs in these experiments.

### Example 7: In Vivo Inhibition of c-JUN Phosphorylation by TAT-IB(s) Peptides as Defined Herein

To determine whether the cell-permeable peptides as defined herein could block JNK signaling in vivo, we used a heterologous GAL4 system. HeLa cells, cultured as described above, were co-transfected with the 5×GAL-LUC reporter vector together with the GAL-Jun expression construct (Stratagene) comprising the activation domain of c-Jun (amino acids 1-89) linked to the GAL4 DNA-binding domain. Activation of JNK was achieved by the co-transfection of vectors expressing the directly upstream kinases MKK4 and MKK7 (see Whitmarsh et al., Science 285: 1573 (1999)). Briefly, 3×10⁵ cells were transfected with the plasmids in 3.5-cm dishes using DOTAP (Boehringer Mannheim) following instructions from the manufacturer. For experiments involving GAL-Jun, 20 ng of the plasmid was transfected with 1 μg of the reporter plasmid pFR-Luc (Stratagene) and 0.5 μg of either MKK4 or MKK7 expressing plasmids. Three hours following transfection, cell media were changed and TAT and TAT-IB1(s) peptides (1 μM) were added. The luciferase activities were measured 16 hours later using the “Dual Reporter System” from Promega after normalization to protein content. Addition of TAT-IB1(s) peptide blocked activation of c-Jun following MKK4 and MKK7 mediated activation of JNK. Because HeLa cells express JNK1 and JNK2 isoforms but not JNK3, we transfected cells with JNK3. Again, the TAT-IB(s) peptide inhibited JNK2 mediated activation of c-Jun.

### Example 8: Synthesis of all-D Retro-Inverso IB(s) Peptides and Variants Thereof

Peptides of the invention may be all-D amino acid peptides synthesized in reverse to prevent natural proteolysis (i.e. all-D retro-inverso peptides). An all-D retro-inverso peptide of the invention would provide a peptide with functional properties similar to the native peptide, wherein the side groups of the component amino acids would correspond to the native peptide alignment, but would retain a protease resistant backbone.

Retro-inverso peptides of the invention are analogs synthesized using D-amino acids by attaching the amino acids in a peptide chain such that the sequence of amino acids in the retro-inverso peptide analog is exactly opposite of that in the selected peptide which serves as the model. To illustrate, if the naturally occurring TAT protein (formed of L-amino acids) has the sequence GRKKRRQRRR [SEQ ID NO: 5], the retro-inverso peptide analog of this peptide (formed of D-amino acids) would have the sequence RRRQRRKKRG [SEQ ID NO: 6]. The procedures for synthesizing a chain of D-amino acids to form the retro-inverso peptides are known in the art (see e.g. Jameson et al., Nature, 368, 744-746 (1994); Brady et al., Nature, 368, 692-693 (1994); Guichard et al., J. Med. Chem. 39, 2030-2039 (1996)). Specifically, the retro-peptides according to SEQ ID NOs 2, 4, 6, 8, 11-12, 18, 20, 22 and 25-26, were produced by classical F-mock synthesis and further analyzed by Mass Spectrometry. They were finally purified by HPLC.

Since an inherent problem with native peptides is degradation by natural proteases and inherent immunogenicity, the heterobivalent or heteromultivalent compounds of this invention will be prepared to include the “retro-inverso isomer” of the desired peptide. Protecting the peptide from natural proteolysis should therefore increase the effectiveness of the specific heterobivalent or heteromultivalent compound, both by prolonging half-life and decreasing the extent of the immune response aimed at actively destroying the peptides.

### Example 9: Long Term Biological Activity of all-D Retro-Inverso IB(s) Peptides and Variants Thereof

Long term biological activity is predicted for the D-TAT-IB(s) retro-inverso containing peptide heteroconjugate (see chimeric sequences above) when compared to the native L-amino acid analog owing to protection of the D-TAT-IB(s) peptide from degradation by native proteases, as shown in Example 5.

Inhibition of IL-1β induced pancreatic beta-cell death by the D-TAT-IB1(s) peptide was analyzed. βTC-3 cells were incubated as described above for 30 minutes with one single addition of the indicated peptides (1 μM), then IL-1 (10 ng/ml) was added.

Apoptotic cells were then counted after two days of incubation with IL-1β by use of Propidium Iodide and Hoechst 33342 nuclear staining. A minimum of 1,000 cells were counted for each experiment. Standard Error of the Means (SEM) are indicated, n=5. The D-TAT-IB1 peptide decreased IL-1 induced apoptosis to a similar extent as L-TAT-IB peptides.

Long term inhibition of IL-1P induced cell-death by the D-TAT-IB1 peptide was also analyzed. βTC-3 cells were incubated as above for 30 minutes with one single addition of the indicated peptides (1 μM), then IL-13 (10 ng/ml) was added, followed by addition of the cytokine every two days. Apoptotic cells were then counted after 15 days of incubation with IL-1 by use of propidium iodide and Hoechst 33342 nuclear staining. Note that one single addition of the TAT-IB1 peptide does not confer long-term protection. A minimum of 1.000 cells were counted for each experiment. As a result, D-TAT-IB(s), but not L-TAT-IB(s), was able to confer long term (15 day) protection.

### Example 10: Suppression of INK Transcription Factors by L-TAT-IB(s) Peptides as Used According to the Present Invention

Gel retardation assays were carried out with an AP-1 doubled labeled probe (5′-CGC TTG ATG AGT CAG CCG GAA-3′ (SEQ ID NO: 101). HeLa cell nuclear extracts that were treated or not for one hour with 5 ng/mlTNF-α, as indicated. TAT and L-TAT-IB1 (s) peptides as used according to the present invention were added 30 minutes before TNF-alpha. Only the part of the gel with the specific AP-1 DNA complex (as demonstrated by competition experiments with non-labeled specific and non-specific competitors) is shown.

L-TAT-IB1(s) peptides as used according to the present invention decrease the formation of the AP-1 DNA binding complex in the presence of TNF-alpha.

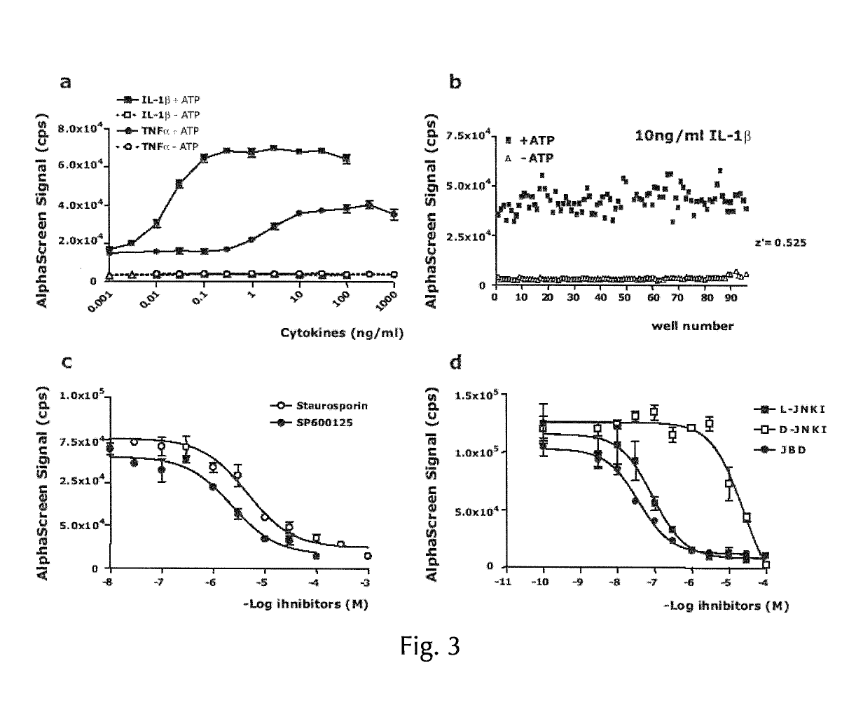

### Example 11: Inhibition of Endogenous INK Activity in HepG2 Cells Using an all-in One Well Approach (See FIG. 3)

HepG2 cells were seeded at 3′000 cells/well the day prior the experiment. Then, increasing concentrations of either interleukin-10 [IL-1 beta v)] or tumor necrosis factor α [TNFalpha] (a) were added to activate JNK for 30 minutes. Cells were lysed in 20 mM Hepes, 0.5% Tween pH 7.4 and processed for AlphaScreen JNK. (b) Z′ for the JNK activity induced by 10 ng/ml IL-1β and measured in 384 wells/plate (n=96). (c) Inhibition of endogenous IL-1 beta-induced JNK activity with chemical JNK inhibitors [staurosporin and SP600125]. (d) Effect of peptidic inhibitors L-TAT-IB1(s) according to SEQ ID NO: 9 [here abbreviated as L-JNKi (v)) and D-TAT-IB1(s) according to SEQ ID NO: 11 (here abbreviated as D-JNKi) and JBDs (corresponds to L-JNKI without the TAT sequence)] on IL-1β dependent JNK activity. All panels are representative of three independent experiments (n=3).

### Methods: Alphascreen Kinase Assay

### Principle:

AlphaScreen is a non-radioactive bead-based technology used to study biomolecular interactions in a microplate format. The acronym ALPHA stands for Amplified Luminescence Proximity Homogenous Assay. It involves a biological interaction that brings a “donor” and an “acceptor” beads in close proximity, then a cascade of chemical reactions acts to produce an amplified signal. Upon laser excitation at 680 nm, a photosensitizer (phthalocyanine) in the “donor” bead converts ambient oxygen to an excited singlet state. Within its 4 μsec half-life, the singlet oxygen molecule can diffuse up to approximately 200 nm in solution and if an acceptor bead is within that proximity, the singlet oxygen reacts with a thioxene derivative in the “acceptor” bead, generating chemiluminescence at 370 nm that further activates fluorophores contained in the same “acceptor” bead. The excited fluorophores subsequently emit light at 520-620 nm. In the absence of an acceptor bead, singlet oxygen falls to ground state and no signal is produced.

Kinase reagents (B-GST-cJun, anti P-cJun antibody and active JNK3) were first diluted in kinase buffer (20 mM Tris-HCl pH 7.6, 10 mM MgCl₂, 1 mM DTT, 100 μM Na₃VO₄, 0.01% Tween-20) and added to wells (15 μl). Reactions were then incubated in presence of 10 μM of ATP for 1 h at 23° C. Detection was performed by an addition of 10 μl of beads mix (Protein A acceptor 20 μg/ml and Streptavidin donor 20 μg/ml), diluted in detection buffer (20 mM Tris-HCl pH 7.4, 20 mM NaCl, 80 mM EDTA, 0.3% BSA), followed by an another one-hour incubation at 23° C. in the dark. For measurement of JNK endogenous activity, kinase assays were performed as described above except active JNK3 was replaced by cells lysates and reaction kinase components were added after the cells lysis. B-GST-cjun and P-cJun antibody were used at the same concentrations whereas ATP was used at 50 μM instead of 10 μM. AlphaScreen signal was analyzed directly on the Fusion or En Vision apparatus.

### Example 12: Effects of the INK Inhibitor According to SEQ ID NO:11 (XG-102) on Endogenous INK Activity in Wildtype and 5XFAD Mice

### 5XFAD Mouse Model of Amyloid Deposition

In order to determine the effects of a JNK inhibitor peptide as described herein, in particular a JNK inhibitor peptide according to SEQ ID NO: 11 (“XG-102”), in Mild Cognitive Impairment, the 5XFAD mouse model was used.

5XFAD mice represent a transgenic mouse model of amyloid deposition. 5XFAD transgenic mice overexpress mutant human APP(695) with five familial AD (FAD) mutations that are additive in driving Aβ42 overproduction, namely the Swedish (K670N, M671L), Florida (1716V), and London (V7171) Familial Alzheimer's Disease (FAD) mutations along with human PS1 harboring two FAD mutations, M146L and L286V.

5XFAD mice are known to exhibit intraneuronal Aβ42 accumulation at 1.5 months, amyloid deposition at 2 months, memory deficits by 4 months of age, and statistically significant neuron loss occurs by 9 months of age. In addition, in young 5XFAD brains, activated Caspase-3 in the soma and proximal dendrites of intraneuronal Aβ42-labeled neurons was observed. In older 5XFAD brains, activated Caspase-3-positive punctate accumulations that co-localize with the neuronal marker class III β-tubulin, suggesting neuron loss by apoptosis, was observed.

In summary, it is of note that 5XFAD mice do not show symptoms of Alzheimer's Disease, such as dementia, from birth—but develop those symptoms during aging. Accordingly, those mice cannot only be used to study Alzheimer's Disease (at an old age), but also for studying Mild Cognitive Impairment due to Alzheimer's Disease (at a younger age). However, for studying Mild Cognitive Impairment, an age is to be selected at which the 5XFAD mice did not yet develop full AD symptomatology.

### Experimental Design and Methods

A total of 60 mice (29 wildtype (WT) and 31 5XFAD) was assigned to eight different groups as follows: Saline 3 months WT (n=8); Saline 3 months 5XFAD (n=7); XG-102 3 months WT (n=8); XG-102 3 months 5XFAD (n=8); Saline 6 months WT (n=7); Saline 6 months 5XFAD (n=7); XG-102 6 months WT (n=6); and XG-102 6 months 5XFAD (n=9).

Two different treatments were carried out: XG-102 (10 mg/kg; administered i.v.; one dose of 10 mg/kg every 3 weeks) and saline (NaCl 0.9%; administration route and schedule corresponding to the XG-102 groups). The duration of treatment was 3 months or 6 months, starting with mice at an age of 3 months. After treatment and testing, mice were sacrificed at an age of 6 months (3-month treatment duration) or 9 months (6-month treatment duration). After sacrifice, brains were removed for analysis.

JNK/p-JNK levels were determined by immunoblot (cortex and hippocampus) using anti-JNK and anti-phospho JNK antibodies. The protein samples (30 to 40 μg) were separated on 4-15% Mini-Protean TGX gels (Biorad Laboratories Inc., Hercules, Calif., USA) and then electroblotted onto nitrocellulose membranes (GE Healthcare). The membranes were blocked in 5% milk in TBS, then incubated with primary antibodies over night, and finally incubated with IR Dye 800 or 700 (Azure Biosystems Inc., Dublin, Calif., USA). Bound proteins were visualized with the Odyssey Imaging System (Li-Cor Biosciences, Lincoln, Nebr., USA). Obtained data were statistically analyzed by two-way ANOVA, followed by a post-hoc multiple comparison test, the Tukey's test (GraphPad Prism).

### Results

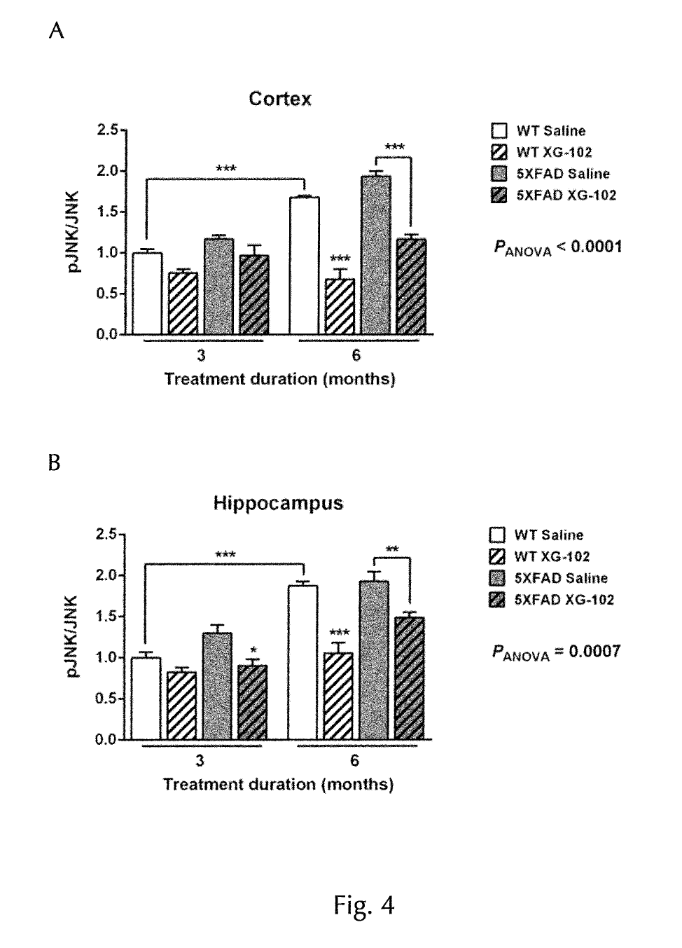

Results are shown in FIG. 4.

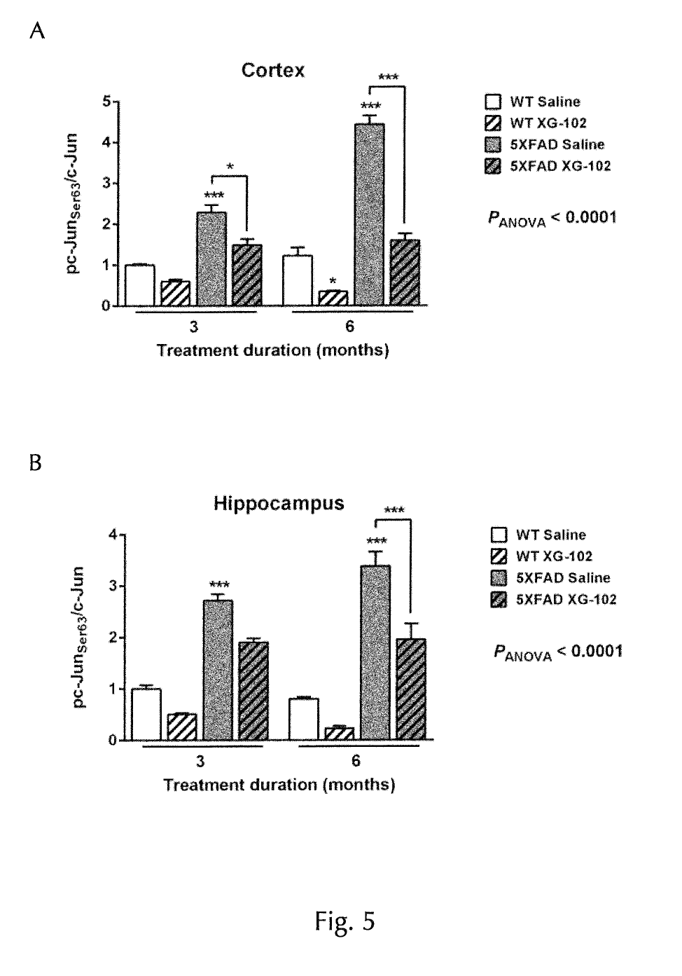

In general, no significant differences in JNK activation (pJNK/JNK ratio) could be detected between 5XFAD and wildtype mice. This shows that 5XFAD mice did not yet develop Alzheimer's Disease symptomatology, since in AD an increase in the pJNK/JNK ratio is to be expected, whereas without any cognitive impairment and in Mild Cognitive Impairment (MCI) no such increased pJNK/JNK ratio is observed (cf. E. J. Mufson et al., 2012, J Neuropathol Exp Neurol 71(11): 1018-1029, in particular FIG. 5 of Mufson et al.).

Both, WT and 5XFAD mice showed an increased JNK activation level (measured by the ratio phosphorylated JNK/JNK full) at an age of 9 months compared to 6 months-old mice of the same genotype. This indicates a generally increased JNK activation during aging, which is independent from Alzheimer's Disease symptomatology since it was also observed in wildtype animals.

XG-102 treatment for 6 months resulted in a decrease of JNK activation levels in the cortex (−59.6% and −39.9%, respectively; FIG. 4A) and in the hippocampus (−43.7% and −22.8%, respectively; FIG. 4B) in both, WT and 5XFAD mice. In addition, a 30.4% decrease of JNK activation levels was observed in the hippocampus of 5XFAD mice treated for 3 months with XG-102 (FIG. 4B).

In summary, these data show that treatment with the JNK peptide inhibitor XG-102 (SEQ ID NO: 11) has a strong inhibitory effect on the JNK activation level in WT and 5XFAD mice.

### Example 13: Effects of the INK Inhibitor According to SEQ ID NO:11 (XG-102) on c-Jun Activity in Wildtype and 5XFAD Mice

### Experimental Design and Methods

The 5XFAD mouse model as described in Example 12 was used.

A total of 60 mice (29 wildtype (WT) and 31 5XFAD) was assigned to eight different groups as follows: Saline 3 months WT (n=8); Saline 3 months 5XFAD (n=7); XG-102 3 months WT (n=8); XG-102 3 months 5XFAD (n=8); Saline 6 months WT (n=7); Saline 6 months 5XFAD (n=7); XG-102 6 months WT (n=6); and XG-102 6 months 5XFAD (n=9).

Two different treatments were carried out: XG-102 (10 mg/kg; administered i.v.; one dose of 10 mg/kg every 3 weeks) and saline (NaCl 0.9%; administration route and schedule corresponding to the XG-102 groups). The duration of treatment was 3 months or 6 months, starting with mice at an age of 3 months. After treatment and testing, mice were sacrificed at an age of 6 months (3-month treatment duration) or 9 months (6-month treatment duration). After sacrifice, brains were removed for analysis.

Pc-Jun_{Ser63}/c-Jun levels were determined by immunoblot (cortex and hippocampus) using anti-cJun (main target of JNK, apoptosis trigger) and anti-phospho cJun [Ser63] (site phosphorylated by JNK) antibodies. The protein samples (30 to 40 μg) were separated on 4-15% Mini-Protean TGX gels (Biorad Laboratories Inc., Hercules, Calif., USA) and then electroblotted onto nitrocellulose membranes (GE Healthcare). The membranes were blocked in 5% milk in TBS, then incubated with primary antibodies over night, and finally incubated with IR Dye 800 or 700 (Azure Biosystems Inc., Dublin, Calif., USA). Bound proteins were visualized with the Odyssey Imaging System (Li-Cor Biosciences, Lincoln, Nebr., USA). Obtained data were statistically analyzed by two-way ANOVA, followed by a post-hoc multiple comparison test, the Tukey's test (GraphPad Prism).

### Results

Results are shown in FIG. 5.

5XFAD mice showed an increase of cJun activation levels at an age of 6 and 9 months, respectively, as compared to WT mice at the corresponding age, in the cortex (+129% and +260%, respectively, FIG. 5A), and in the hippocampus (+172% and +321%, respectively; FIG. 5B). XG-102 treatment for 3 months in 5XFAD mice showed a 34.9% decrease of cJun activation level in the cortex. A 6 months treatment in 5XFAD mice showed a strong decrease of cJun activation level in the cortex and in the hippocampus (−64% and −42.1%, respectively).

### Example 14: Effects of the INK Inhibitor According to SEQ ID NO:11 (XG-102) on Amyloid β Load in Wildtype and 5XFAD Mice

Experimental Design and Methods

The 5XFAD mouse model as described in Example 12 was used.

3-month old 15 5XFAD mice were assigned to two different groups Saline 5XFAD (n=7); and XG-102 5XFAD (n=8). Accordingly, 5XFAD mice were either treated with XG-102 (10 mg/kg; administered i.v.; one dose of 10 mg/kg every 3 weeks) or with saline (NaCl 0.9%; administration route and schedule corresponding to the XG-102 groups) for 3 months. After treatment and testing, mice were sacrificed (at an age of 6 months). After sacrifice, brains were removed for analysis.

In order to determine the levels of amyloid β3 42 (Aβ42) in the layer 5 of the cortex, sagittal brain sections from mice were incubated with two anti-human Aβ42 clone monoclonal antibody (monoclonal mouse anti-human Aβ42 clone 6F/3D, Dako North America Inc, CA, USA, also link APP and monoclonal mouse anti-human Aβ42 clone 6C3 (MOAB-2), Millipore, Billerica, USA, specific to Aβ42). Paraffined brains were sagitally sectioned on a microtome apparatus at 5 μm. Sections were deparaffinized in xylene and rehydrated in descending concentrations of ethanol. Sections were heated in citrate buffer, then treated with hydrogen peroxide. Sections were treated in blocking solution before being incubated over night with primary antibodies. Biotinylated anti-rabbit and anti-mouse (Vector Laboratory, Bar Harbor, Me., USA) were used as secondary antibodies. Quantification of staining (% area stained of total area examined) was performed in Layer 5 cortex and subiculum by ImageJ 1.48v software (developed by National Institutes of Health, USA). Obtained data were statistically analyzed by two-way ANOVA, followed by a post-hoc multiple comparison test, the Tukey's test (GraphPad Prism).

### Results

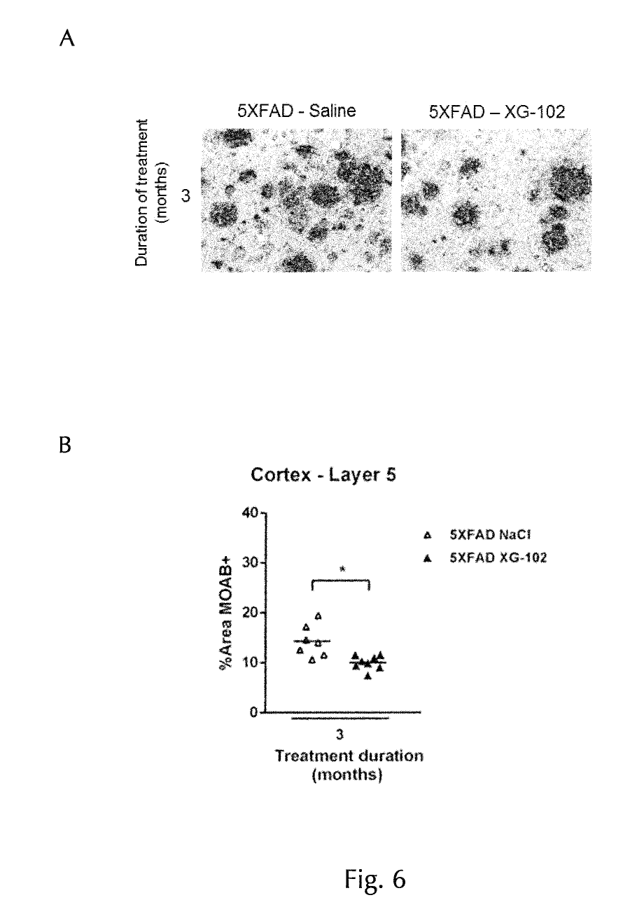

Results are shown in FIG. 6. A 29.9% decrease of amyloid burden was observed in 5XFAD mice treated by XG-102 as compared to 5XFAD mice treated with saline.

### Example 15: Effects of the INK Inhibitor According to SEQ ID NO:11 (XG-102) on pAPP Level in Wildtype and 5XFAD Mice

Experimental Design and Methods

The 5XFAD mouse model as described in Example 12 was used.

A total of 60 mice (29 wildtype (WT) and 31 5XFAD) was assigned to eight different groups as follows: Saline 3 months WT (n=8); Saline 3 months 5XFAD (n=7); XG-102 3 months WT (n=8); XG-102 3 months 5XFAD (n=8); Saline 6 months WT (n=7); Saline 6 months 5XFAD (n=7); XG-102 6 months WT (n=6); and XG-102 6 months 5XFAD (n=9).

Two different treatments were carried out: XG-102 (10 mg/kg; administered i.v.; one dose of 10 mg/kg every 3 weeks) and saline (NaCl 0.9%; administration route and schedule corresponding to the XG-102 groups). The duration of treatment was 3 months or 6 months, starting with mice at an age of 3 months. After treatment and testing, mice were sacrificed at an age of 6 months (3-month treatment duration) or 9 months (6-month treatment duration). After sacrifice, brains were removed for analysis.

Levels of pAPP (phosphorylated amyloid precursor protein) in the hippocampus were determined by immunoblot using a rabbit monoclonal anti-pAPP [Thr668], clone D90B8 (Cell Signaling, Danvers, USA). The protein samples (30 to 40 μg) were separated on 4-15% Mini-Protean TGX gels (Biorad Laboratories Inc., Hercules, Calif., USA) and then electroblotted onto nitrocellulose membranes (GE Healthcare). The membranes were blocked in 5% milk in TBS, then incubated with primary antibodies over night, and finally incubated with IR Dye 800 or 700 (Azure Biosystems Inc., Dublin, Calif., USA). Bound proteins were visualized with the Odyssey Imaging System (Li-Cor Biosciences, Lincoln, Nebr., USA). Obtained data were statistically analyzed by two-way ANOVA, followed by a post-hoc multiple comparison test, the Tukey's test (GraphPad Prism).

### Results

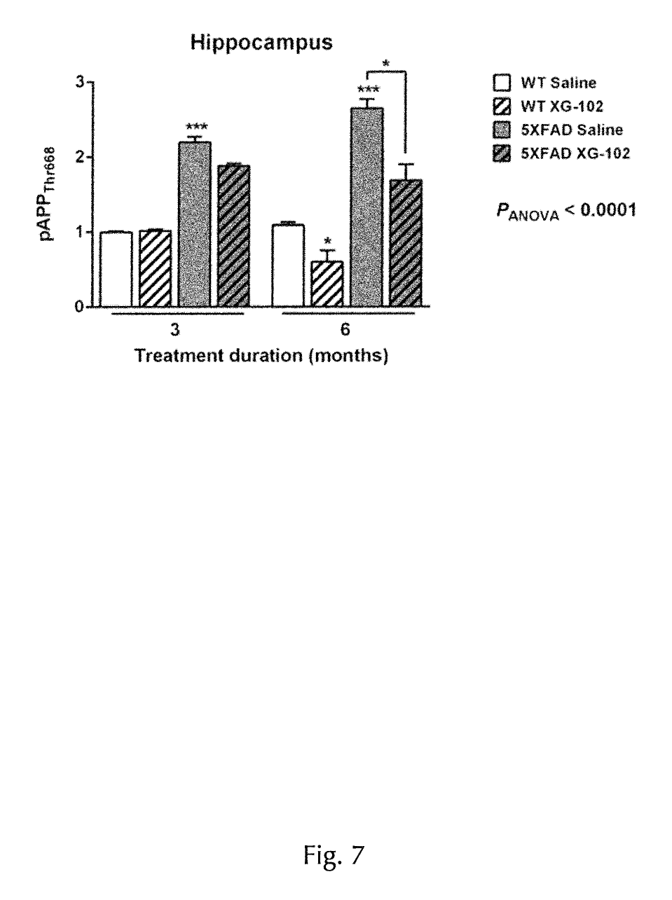

Results are shown in FIG. 7. Levels of pAPP in the hippocampus of 5XFAD mice treated with XG-102 showed a slight decrease after 3 months of treatment and a strong decrease of 36.2% after 6 months of treatment as compared to saline-treated 5XFAD mice.

### Example 16: Effects of the INK Inhibitor According to SEQ ID NO:11 (XG-102) on Activated Caspase 3 Levels in Wildtype and 5XFAD Mice

Experimental Design and Methods

The 5XFAD mouse model as described in Example 12 was used.

A total of 60 mice (29 wildtype (WT) and 31 5XFAD) was assigned to eight different groups as follows: Saline 3 months WT (n=8); Saline 3 months 5XFAD (n=7); XG-102 3 months WT (n=8); XG-102 3 months 5XFAD (n=8); Saline 6 months WT (n=7); Saline 6 months 5XFAD (n=7); XG-102 6 months WT (n=6); and XG-102 6 months 5XFAD (n=9).

Two different treatments were carried out: XG-102 (10 mg/kg; administered i.v.; one dose of 10 mg/kg every 3 weeks) and saline (NaCl 0.9%; administration route and schedule corresponding to the XG-102 groups). The duration of treatment was 3 months or 6 months, starting with mice at an age of 3 months. After treatment and testing, mice were sacrificed at an age of 6 months (3-month treatment duration) or 9 months (6-month treatment duration). After sacrifice, brains were removed for analysis.

Levels of activated caspase 3 (cleaved caspase 3) in the cortex and hippocampus, were determined by immunoblot on sagittal brain sections from mice incubated with an anti-cleaved caspase 3 antibody (Rabbit polyclonal anti-cleaved caspase 3, Cell Signaling, Danvers, USA). The protein samples (30 to 40 μg) were separated on 4-15% Mini-Protean TGX gels (Biorad Laboratories Inc., Hercules, Calif., USA) and then electroblotted onto nitrocellulose membranes (GE Healthcare). The membranes were blocked in 5% milk in TBS, then incubated with primary antibodies over night, and finally incubated with IR Dye 800 or 700 (Azure Biosystems Inc., Dublin, Calif., USA). Bound proteins were visualized with the Odyssey Imaging System (Li-Cor Biosciences, Lincoln, Nebr., USA). Obtained data were statistically analyzed by two-way ANOVA, followed by a post-hoc multiple comparison test, the Tukey's test (GraphPad Prism).

Results

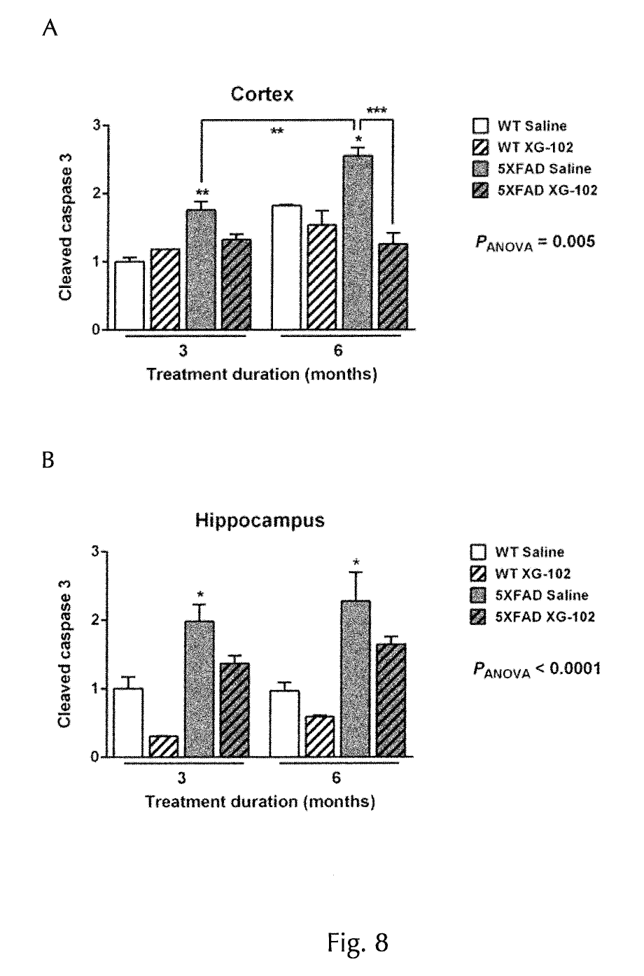

Results are shown in FIG. 8. The levels of the activated form of caspase 3 (cleaved caspase 3) are significantly increased in the cortex (FIG. 8A) and in the hippocampus (FIG. 8B) of saline-treated 5XFAD mice at an age of 6 and 9 months, respectively (+75% and +40%, respectively, in the cortex, and +98% and +36%, respectively, in the hippocampus) as compared to WT mice of corresponding age. Treatment with XG-102, however, reduced activated caspase 3 levels in 5XFAD mice with the strongest decrease (of 50.8% of cleaved caspase 3 level) observed in the cortex of 5XFAD mice treated with XG-102 for 6 months.

### Example 17: Effects of the JNK Inhibitor According to SEQ ID NO:11 (XG-102) on pBcl2 (Ser871 Levels in Wildtype and 5XFAD Mice

Experimental Design and Methods

The 5XFAD mouse model as described in Example 12 was used.

A total of 60 mice (29 wildtype (WT) and 31 5XFAD) was assigned to eight different groups as follows: Saline 3 months WT (n=8); Saline 3 months 5XFAD (n=7); XG-102 3 months WT (n=8); XG-102 3 months 5XFAD (n=8); Saline 6 months WT (n=7); Saline 6 months 5XFAD (n=7); XG-102 6 months WT (n=6); and XG-102 6 months 5XFAD (n=9).

Two different treatments were carried out: XG-102 (10 mg/kg; administered i.v.; one dose of 10 mg/kg every 3 weeks) and saline (NaCl 0.9%; administration route and schedule corresponding to the XG-102 groups). The duration of treatment was 3 months or 6 months, starting with mice at an age of 3 months. After treatment and testing, mice were sacrificed at an age of 6 months (3-month treatment duration) or 9 months (6-month treatment duration). After sacrifice, brains were removed for analysis.

Levels of pBcl2 [Ser87] in the cortex and hippocampus were determined by immunoblot on sagittal brain sections from mice incubated with an anti-pBcl2 [Ser87] antibody (mouse monoclonal anti-pBcl-2 [Ser87], clone C-2, Santa Cruz, Danvers, USA). The protein samples (30 to 40 μg) were separated on 4-15% Mini-Protean TGX gels (Biorad Laboratories Inc., Hercules, Calif., USA) and then electroblotted onto nitrocellulose membranes (GE Healthcare). The membranes were blocked in 5% milk in TBS, then incubated with primary antibodies over night, and finally incubated with IR Dye 800 or 700 (Azure Biosystems Inc., Dublin, Calif., USA). Bound proteins were visualized with the Odyssey Imaging System (Li-Cor Biosciences, Lincoln, Nebr., USA). Obtained data were statistically analyzed by two-way ANOVA, followed by a post-hoc multiple comparison test, the Tukey's test (GraphPad Prism).

Results

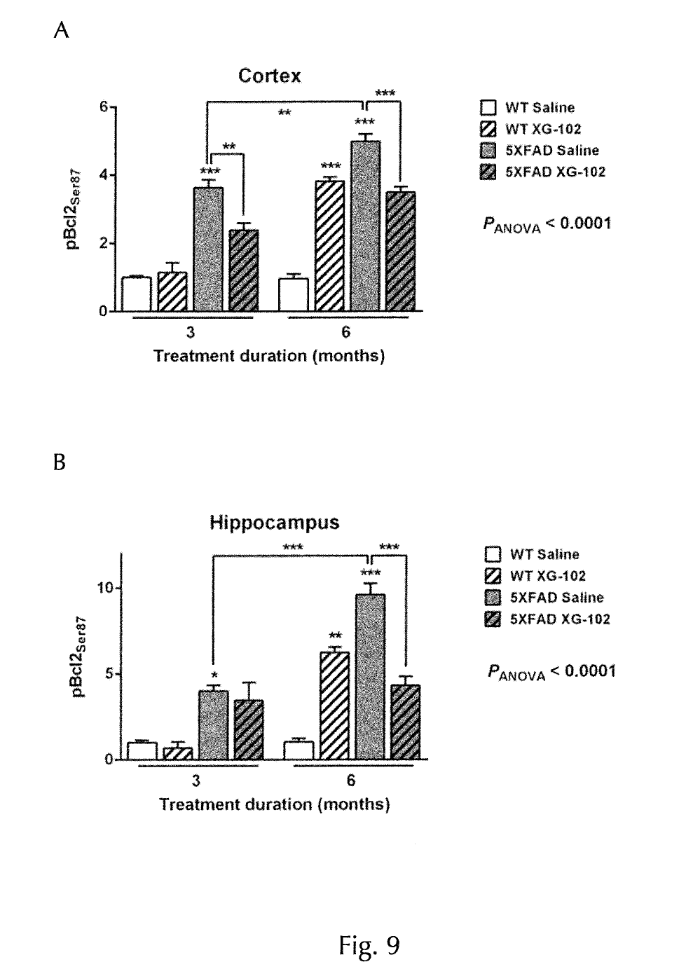

Results are shown in FIG. 9. Phosphorylation of Bcl2 on serine 87 decreases the anti-apoptotic role of Bcl2. The levels of Bcl2 phosphorylated on serine 87 (pBCL [Ser87]) are significantly increased in the cortex (FIG. 9A) and hippocampus (FIG. 9B) of saline-treated 5XFAD mice at an age of 6 and 9 months, (+263% and +415% in the cortex, respectively, and +302% and +807%, respectively, in the hippocampus) as compared to WT mice of corresponding age. Treatment with XG-102, however, pronouncedly decreased pBCL [Ser87] levels in 5XFAD mice.

### Example 18: Effects of the INK Inhibitor According to SEQ ID NO:11 (XG-102) on Caspase 3 Activity in Wildtype and 5XFAD Mice

Experimental Design and Methods

The 5XFAD mouse model as described in Example 12 was used.

A total of 60 mice (29 wildtype (WT) and 31 5XFAD) was assigned to eight different groups as follows: Saline 3 months WT (n=8); Saline 3 months 5XFAD (n=7); XG-102 3 months WT (n=8); XG-102 3 months 5XFAD (n=8); Saline 6 months WT (n=7); Saline 6 months 5XFAD (n=7); XG-102 6 months WT (n=6); and XG-102 6 months 5XFAD (n=9).

Two different treatments were carried out: XG-102 (10 mg/kg; administered i.v.; one dose of 10 mg/kg every 3 weeks) and saline (NaCl 0.9%; administration route and schedule corresponding to the XG-102 groups). The duration of treatment was 3 months or 6 months, starting with mice at an age of 3 months. After treatment and testing, mice were sacrificed at an age of 6 months (3-month treatment duration) or 9 months (6-month treatment duration). After sacrifice, brains were removed for analysis.

In order to determine the activity of caspase 3 in the cortex and hippocampus, an enzyme assay was performed. Caspase 3 activity was measured by using the Caspase 3 Assay kit reagents and protocol (Abcam, Cambridge, UK). Caspase 3 Assay Kit was based on spectrophotometric detection of the chromophore p-nitroaniline (p-NA) after cleavage by caspase 3 from the labeled substrate DEVD-pNA. The p-NA light emission could be quantified using a spectrophotometer or a microtiter plate reader at 400- or 405 nm. Obtained data were statistically analyzed by two-way ANOVA, followed by a post-hoc multiple comparison test, the Tukey's test (GraphPad Prism).

Results

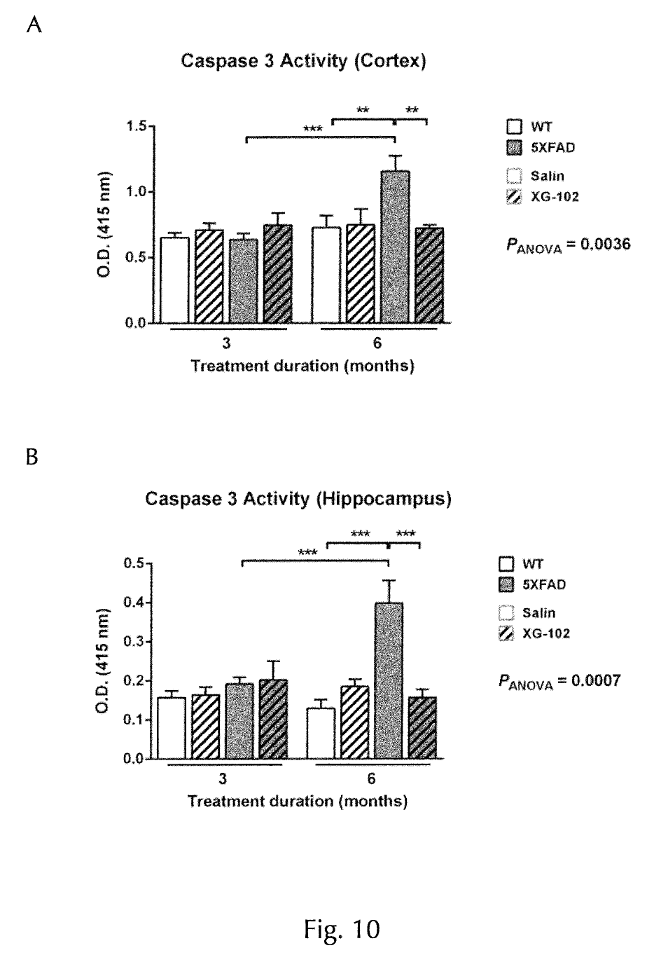

Results are shown in FIG. 10. After 3 months of treatment (6 month old mice), no differences were observed between the groups—neither between 5XFAD and wildtype mice, nor between saline or XG-102 treatment. In 9-months old 5XFAD mice, however, caspase 3 activity was significantly increased in both, cortex (+59%, FIG. 10A) and hippocampus (+207%, FIG. 10B) as compared to wildtype animals at a corresponding age. Treatment with XG-102 reversed this effect in 5XFAD mice and resulted in a significant decrease of caspase 3 activity (−37.7% and −60.6%, respectively, in the cortex and hippocampus) as compared to saline-treated 5XFAD mice.

In summary, it can be concluded that levels of activated form and activity of caspase 3, which is implicated in neuronal apoptosis, and the level of pBcl2[Ser87] are increased in 5XFAD mice. Treatment with XG-102 is efficient to decrease both expression and activity levels of caspase 3 as well as the expression level of pBcl2 [Ser87]. Accordingly, a treatment with XG-102 is efficient in decreasing important neuronal death pathways. In conclusion, XG-102 has an efficient neuroprotective effect in vivo.

### Example 19: Effects of the INK Inhibitor According to SEQ ID NO:11 (XG-102) on IL1-β Levels in Wildtype and 5XFAD Mice

In order to assess the level of neuroinflammation, XG-102 effects on the cytokine IL1-β were determined in 5XFAD and wildtype mice.

Experimental Design and Methods

The 5XFAD mouse model as described in Example 12 was used.

A total of 60 mice (29 wildtype (WT) and 31 5XFAD) was assigned to eight different groups as follows: Saline 3 months WT (n=8); Saline 3 months 5XFAD (n=7); XG-102 3 months WT (n=8); XG-102 3 months 5XFAD (n=8); Saline 6 months WT (n=7); Saline 6 months 5XFAD (n=7); XG-102 6 months WT (n=6); and XG-102 6 months 5XFAD (n=9).

Two different treatments were carried out: XG-102 (10 mg/kg; administered i.v.; one dose of 10 mg/kg every 3 weeks) and saline (NaCl 0.9%; administration route and schedule corresponding to the XG-102 groups). The duration of treatment was 3 months or 6 months, starting with mice at an age of 3 months. After treatment and testing, mice were sacrificed at an age of 6 months (3-month treatment duration) or 9 months (6-month treatment duration). After sacrifice, brains were removed for analysis.

In order to determine the level of the cytokine IL1-(in the cortex, a LUMINEX assay was performed. The sample was added to 6.5 μm magnetic beads, pre-coated with the specific capture antibody. The antibody binds to the analyte of interest. Biotinylated detection antibody specific to the analyte was added and, then the Phycoerythrin (PE)-conjugated streptavidin. Beads were read on a MagPix instrument where two spectrally distinct light-emitting diodes (LEDs) illuminated the beads. One LED identified the analyte and, the second LED determined the magnitude of the PE-derived signal. Obtained data were statistically analyzed by two-way ANOVA, followed by a post-hoc multiple comparison test, the Tukey's test (GraphPad Prism).

Results

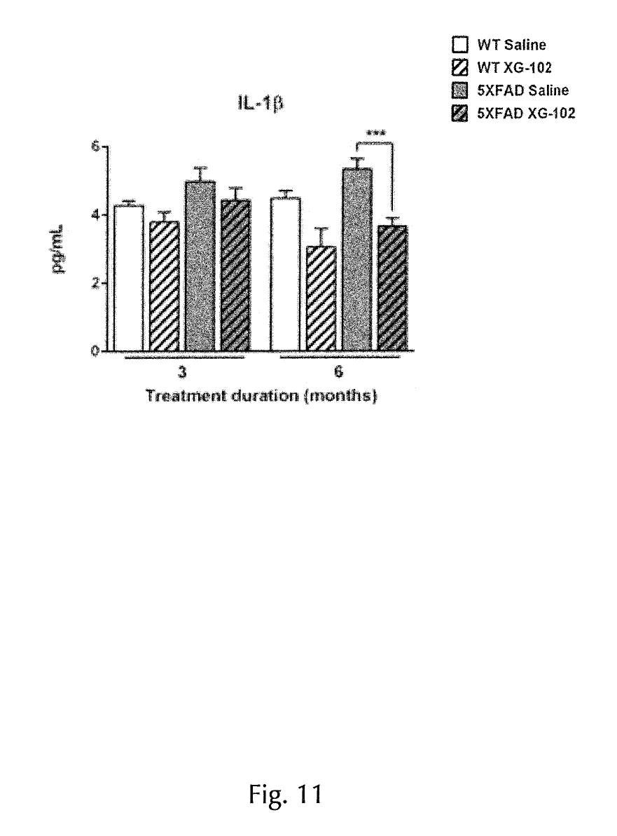

Results are shown in FIG. 11. After 3 months of treatment (6 month old mice), no differences were observed between the groups—neither between 5XFAD and wildtype mice, nor between saline or XG-102 treatment. After 6 months of treatment, however, a 31.4% decrease of IL-1β expression level in 5XFAD mice treated with XG-102 was observed (as compared to saline-treated 5XFAD mice).

### Example 20: Effects of the JNK Inhibitor According to SEQ ID NO:11 (XG-102) on Spatial and Working Memory in Wildtype and 5XFAD Mice

In order to study spatial and procedural working memory (hippocampus), mice were tested in the Y-maze task.

Experimental Design and Methods

The 5XFAD mouse model as described in Example 12 was used.

A total of 60 mice (29 wildtype (WT) and 31 5XFAD) was assigned to eight different groups as follows: Saline 3 months WT (n=8); Saline 3 months 5XFAD (n=7); XG-102 3 months WT (n=8); XG-102 3 months 5XFAD (n=8); Saline 6 months WT (n=7); Saline 6 months 5XFAD (n=7); XG-102 6 months WT (n=6); and XG-102 6 months 5XFAD (n=9).

Two different treatments were carried out: XG-102 (10 mg/kg; administered i.v.; one dose of 10 mg/kg every 3 weeks) and saline (NaCl 0.9%; administration route and schedule corresponding to the XG-102 groups). The duration of treatment was 3 months or 6 months, starting with mice at an age of 3 months. After treatment and testing, mice were sacrificed at an age of 6 months (3-month treatment duration) or 9 months (6-month treatment duration). After sacrifice, brains were removed for analysis.

In order to determine the spatial and working memory, Y-maze spontaneous alternation was used. Y Maze spontaneous alternation is a behavioral test for measuring the willingness of rodents to explore new environments. Rodents, such as mice, typically prefer to investigate a new arm of the maze rather than returning to one that was previously visited. Over the course of multiple arm entries, the mouse should show a tendency to enter a less recently visited arm. The number of arm entries and the number of triads are recorded in order to calculate the percentage of alternation. Many parts of the brain—including the hippocampus, septum, basal forebrain, and prefrontal cortex—are involved in this task.

To test Y-maze spontaneous alternation, each mouse was placed in the centre of the Y-maze. The Y-maze has 3 equal arms of 27 cm length, 7 cm width, and 20 cm height. During 8 min testing, the spontaneous alternation behaviour between the three arms of the maze was measured. Percentage alternation corresponded to the number of triads entries divided by the total number of arms entered minus 2 multiplied 100. Obtained data were statistically analyzed by two-way ANOVA, followed by a post-hoc multiple comparison test, the Tukey's test (GraphPad Prism).

Results

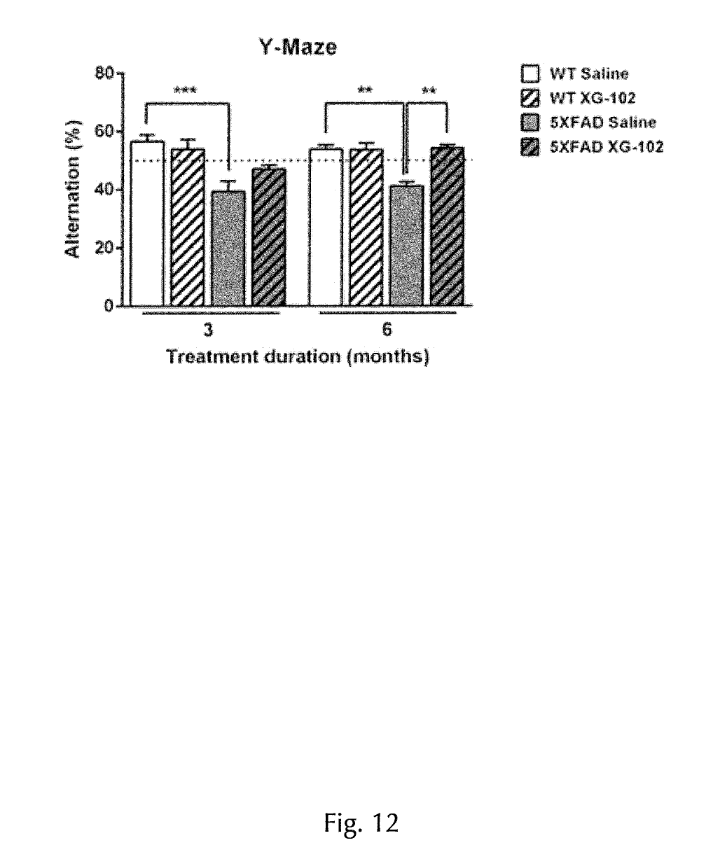

Results are shown in FIG. 12. A significant decrease of the spatial and procedural working memory of 5XFAD mice at an age of 6 and 9 months was observed as compared to wildtype mice of a corresponding age (−30.3% and −23.8%, respectively). 5XFAD mice treated for 6 months with XG-102 presented a 32% improvement of their spatial and procedural memory.

1. JNK inhibitor sequence comprising less than 150 amino acids in length for use in preventing and/or treating Mild Cognitive Impairment, in particular Mild Cognitive Impairment due to Alzheimer's Disease.

2. The JNK inhibitor sequence for use according to claim 1, wherein the JNK inhibitor sequence comprises a range of 5 to 150 amino acid residues, more preferably 10 to 100 amino acid residues, even more preferably 10 to 75 amino acid residues and most preferably a range of 10 to 50 amino acid residues.

3. The JNK inhibitor sequence for use according to claim 1, wherein the JNK inhibitor sequence binds c-jun N-terminal kinase (JNK).

4. The JNK inhibitor sequence for use according to claim 1, wherein the JNK inhibitor sequence inhibits the activation of at least one JNK targeted transcription factor when the JNK inhibitor sequence is present in a JNK expressing cell.

5. The JNK inhibitor sequence for use according to claim 4, wherein the JNK targeted transcription factor is selected from the group consisting of c-Jun, ATF2, and Elk1.

6. The JNK inhibitor sequence for use according to claim 1, wherein the JNK inhibitor sequence alters a JNK effect when the peptide is present in a JNK expressing cell.

7. The JNK inhibitor sequence for use according to claim 1, wherein the JNK inhibitor sequence is composed of L-amino acids, D-amino acids, or a combination of both, preferably comprises at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the JNK inhibitor sequences in a blockwise, a non-blockwise or in an alternate manner.

8. The JNK inhibitor sequence for use according to claim 1, wherein the JNK inhibitor sequence comprises a fragment, variant, or variant of such fragment of a human or rat IB1 sequence as defined or encoded by any of sequences according to SEQ ID NO: 102, SEQ ID NO: 103, SEQ ID NO: 104 or SEQ ID NO: 105.

9. The JNK inhibitor sequence for use according to claim 1, wherein the JNK inhibitor sequence comprises or consists of at least one amino acid sequence according to SEQ ID NOs: 1 to 4, 13 to 20 and 33 to 100, or a fragment, derivative or variant thereof.

10. A chimeric peptide comprising at least one first domain and at least one second domain linked by a covalent bond, the first domain comprising a trafficking sequence, and the second domain comprising a JNK inhibitor sequence as defined in claim 1 for use in preventing and/or treating Mild Cognitive Impairment, in particular Mild Cognitive Impairment due to Alzheimer's Disease.

11. The chimeric peptide for use according to claim 10, wherein the chimeric peptide is composed of L-amino acids, D-amino acids, or a combination of both, preferably comprises at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the chimeric peptide in a blockwise, a non-blockwise or in an alternate manner.

12. The chimeric peptide for use according to claim 10, wherein the trafficking sequence comprises the amino acid sequence of a human immunodeficiency virus TAT polypeptide.

13. The chimeric peptide for use according to claim 10, wherein the trafficking sequence consists of or comprises the amino acid sequence of SEQ ID NO: 5, 6, 7, 8, 21 or 22.

14. The chimeric peptide for use according to claim 10, wherein the trafficking sequence augments cellular uptake of the peptide.

15. The chimeric peptide for use according to claim 10, wherein the trafficking sequence directs nuclear localization of the peptide.

16. The chimeric peptide for use according to claim 10, wherein the chimeric peptide consists of or comprises the amino acid sequence of any of SEQ ID NOs: 9 to 12 and 23 to 32, or a fragment, or variant thereof.

17. The chimeric peptide for use according to claim 10, wherein the chimeric peptide consists of or comprises an amino acid sequence having at least 70%, preferably at least 80%, more preferably at least 90%, even more preferably at least 95%, and most preferably at least 98% sequence identity to SEQ ID NO: 9 or 11.

18. The chimeric peptide for use according to claim 17, wherein the chimeric peptide consists of or comprises the amino acid sequence of SEQ ID NO: 9 or 11.

19. The chimeric peptide for use according to claim 17, wherein the chimeric peptide consists of or comprises
(i) the amino acid sequence of SEQ ID NO: 11; or
(ii) an amino acid sequence having at least 70%, preferably at least 80%, more preferably at least 90%, even more preferably at least 95%, and most preferably at least 98% sequence identity to SEQ ID NO: 11.

20. The chimeric peptide for use according to claim 10, wherein
(i) the C-terminus of the chimeric peptide is modified by an amide modification; and/or
(ii) the N-terminus of the chimeric peptide is modified by an NH₂-protection group, such as acylation.

21. A combination of
a) the JNK inhibitor sequence as defined in claim 1; and
b) a PKR inhibitor
for use in preventing and/or treating Mild Cognitive Impairment, in particular Mild Cognitive Impairment due to Alzheimer's Disease.

22. The combination for use according to claim 21, wherein the combination further comprises
(c) an amyloid lowering agent; and/or
(d) a glucocorticoid.

23. A combination of
a) the JNK inhibitor sequence as defined in claim 1; and
b) an amyloid lowering agent
for use in preventing and/or treating Mild Cognitive Impairment, in particular Mild Cognitive Impairment due to Alzheimer's Disease.

24. The combination for use according to claim 23, wherein the combination further comprises
(c) a PKR inhibitor; and/or
(d) a glucocorticoid.

25. The combination for use according to claim 21, wherein the JNK inhibitor sequence or the chimeric peptide is administered before or after the PKR inhibitor.

26. The combination for use according to claim 21, wherein the JNK inhibitor sequence or the chimeric peptide is administered via the same or a distinct route of administration as the PKR inhibitor.

27. A pharmaceutical composition comprising the JNK inhibitor sequence as defined in claim 1 and a pharmaceutically acceptable carrier for use in preventing and/or treating Mild Cognitive Impairment, in particular Mild Cognitive Impairment due to Alzheimer's Disease.

28. The pharmaceutical composition for use according to claim 27, wherein the pharmaceutical composition further comprises a PKR inhibitor.

29. The pharmaceutical composition for use according to claim 27, wherein the pharmaceutical composition further comprises an amyloid lowering agent and/or a glucocorticoid.

30. The JNK inhibitor sequence for use according to claim 1, wherein the Mild Cognitive Impairment is amnestic Mild Cognitive Impairment (a-MCI) or non-amnestic Mild Cognitive Impairment (na-MCI), preferably the Mild Cognitive Impairment is amnestic Mild Cognitive Impairment (a-MCI), more preferably the Mild Cognitive Impairment is Mild Cognitive Impairment due to Alzheimer's Disease.

31. An isolated nucleic acid encoding a JNK inhibitor sequence as defined in claim 1 for use in preventing and/or treating Mild Cognitive Impairment, in particular Mild Cognitive Impairment due to Alzheimer's Disease.

32. A vector comprising the nucleic acid as defined in claim 31 for use in preventing and/or treating Mild Cognitive Impairment, in particular Mild Cognitive Impairment due to Alzheimer's Disease.

33. A cell comprising the isolated nucleic acid as defined in claim 31 for use in preventing and/or treating Mild Cognitive Impairment, in particular Mild Cognitive Impairment due to Alzheimer's Disease.

34. An antibody which binds immunospecifically to a JNK inhibitor sequence as defined in claim 1 for use in preventing and/or treating Mild Cognitive Impairment, in particular Mild Cognitive Impairment due to Alzheimer's Disease.

35. The JNK inhibitor sequence for use according to claim 1 wherein the JNK inhibitor, the chimeric peptide or the pharmaceutical composition is to be administered by an administration route selected from the group consisting of (i) parenteral routes, including intravenous, intramuscular, subcutaneous, intradermal, transdermal; (ii) enteral routes, including orally, rectally; (iii) topical routes, including nasal, intranasal; (iv) administration routes avoiding the blood brain barrier, including intra-CSF, intrathecal; and (v) other routes, including epidermal or patch delivery.

36. The JNK inhibitor sequence for use according to claim 1, wherein a dose (per kg bodyweight) of the JNK inhibitor sequence and/or chimeric peptide is in the range of up to 10 mmol/kg, preferably up to 1 mmol/kg, more preferably up to 100 μmol/kg, even more preferably up to 10 μmol/kg, even more preferably up to 1 μmol/kg, even more preferably up to 100 nmol/kg, most preferably up to 50 nmol/kg.

37. The JNK inhibitor sequence for use according to claim 1, wherein a dose (per kg bodyweight) of the JNK inhibitor sequence and/or chimeric peptide is in the range of up to 100 mg/kg, preferably up to 50 mg/kg, more preferably up to 10 mg/kg, and most preferably up to 1 mg/kg.

38. The JNK inhibitor sequence for use according to claim 1, wherein a dose of the JNK inhibitor sequence and/or chimeric peptide in the range of from about 1 pmol/kg to about 1 mmol/kg, from about 10 pmol/kg to about 0.1 mmol/kg, from about 10 pmol/kg to about 0.01 mmol/kg, from about 50 pmol/kg to about 1 μmol/kg, from about 100 pmol/kg to about 500 nmol/kg, from about 200 pmol/kg to about 300 nmol/kg, from about 300 pmol/kg to about 100 nmol/kg, from about 500 pmol/kg to about 50 nmol/kg, from about 750 pmol/kg to about 30 nmol/kg, from about 250 pmol/kg to about 5 nmol/kg, from about 1 nmol/kg to about 10 nmol/kg, or a combination of any two of said values.

39. The JNK inhibitor sequence for use according to claim 1, wherein a dose (per kg bodyweight) of the JNK inhibitor sequence and/or chimeric peptide is in the range of 1 μg/kg to 100 mg/kg, preferably 10 μg/kg to 50 mg/kg, more preferably 100 μg/kg to 10 mg/kg, and most preferably 500 μg/kg to 1 mg/kg.

40. The JNK inhibitor sequence for use according to claim 1, wherein the JNK inhibitor sequence and/or the chimeric peptide is administered repeatedly, preferably at least once per month, at least once every three weeks, at least once every two weeks, or at least once per week; more preferably at least once per month, at least once every three weeks, or at least once every two weeks; even more preferably at least once per month or at least once every three weeks; most preferably at least once every three weeks.

41. Use of the JNK inhibitor sequence as defined in claim 1 for preventing and/or treating Mild Cognitive Impairment, in particular Mild Cognitive Impairment due to Alzheimer's Disease.

42. Method of preventing and/or treating Mild Cognitive Impairment, in particular Mild Cognitive Impairment due to Alzheimer's Disease, in a subject in need thereof comprising administering to the subject the JNK inhibitor sequence as defined in claim 1.

43. The method of claim 42, wherein the JNK inhibitor is administered to the subject.

44. The method of claim 42, wherein the subject was diagnosed with Mild Cognitive Impairment, preferably with amnestic or non-amnestic Mild Cognitive Impairment, more preferably with amnestic Mild Cognitive Impairment, even more preferably with Mild Cognitive Impairment due to Alzheimer's Disease.

45. (canceled)


SEQUENCE LISTING (105 sequences)

SEQ ID NO: 1 - protein, 19 aa, Artificial
  Description of sequence Peptide L-IB1(s) (see Table 1)
RPKRPTTLNLFPQVPRSQD

SEQ ID NO: 2 - protein, 19 aa, Artificial
  Description of sequence Peptide D-IB1(s) (see Table 1)
  MUTAGEN (1)..(19): all amino acids are D-amino acids
DQSRPVQPFLNLTTPRKPR

SEQ ID NO: 3 - protein, 19 aa, Artificial
  Description of sequence Peptide L-IB (generic) (s) (see Table 1)
  misc_feature: Description of sequence Description of sequence general formula NH2-Xnb-Xna-RPTTLXLXXXXXXXQD-Xnb-COOH (see Table 1)
  VARIANT (1)..(1): Xaa is Xnb as defined in the general formula, wherein Xaa represents an amino acid residue, preferably selected from any (native) amino acid residue;
  REPEAT (1)..(1): Xaa is Xnb as defined in the general formula, wherein n is 0-5, 5-10, 10-15, 15-20, 20-30 or more for Xnb
  VARIANT (2)..(2): Xaa is Xna as defined in the general formula, wherein Xaa represents an amino acid residue, preferably selected from any (native) amino acid residue residue except serine and threonine
  REPEAT (2)..(2): Xaa is Xna as defined in the general formula, wherein n is 0 or 1
  VARIANT (8)..(8): Xaa represents an amino acid residue, preferably selected from any (native) amino acid residue;
  VARIANT (10)..(16): Xaa represents an amino acid residue, preferably selected from any (native) amino acid residue;
  REPEAT (19)..(19): Xaa is Xnb as defined in the general formula, wherein n is 0-5, 5-10, 10-15, 15-20, 20-30 or more for Xnb
  VARIANT (19)..(19): Xaa is Xnb as defined in the general formula, wherein Xaa represents an amino acid residue, preferably selected from any (native) amino acid residue;
XXRPTTLXLXXXXXXXQDX

SEQ ID NO: 4 - protein, 19 aa, Artificial
  Description of sequence Peptide D-IB (generic) (s) (see Table 1)
  misc_feature: Description of sequence general formula NH2-Xnb-DQXXXXXXXLXLTTPR-Xna-Xnb-COOH,
  MUTAGEN (1)..(19): all amino acids are D-amino acids
  VARIANT (1)..(11): Xaa is Xnb as defined in the general formula, wherein Xaa represents an amino acid residue, preferably selected from any (native) amino acid residue;
  REPEAT (1)..(1): Xaa is Xnb as defined in the general formula, wherein n is 0-5, 5-10, 10-15, 15-20, 20-30 or more for Xnb
  VARIANT (4)..(10): Xaa represents an amino acid residue, preferably selected from any (native) amino acid residue;
  VARIANT (12)..(12): Xaa represents an amino acid residue, preferably selected from any (native) amino acid residue;
  REPEAT (18)..(18): Xaa is Xna as defined in the general formula, wherein n is 0 or 1
  VARIANT (18)..(18): Xaa is Xna as defined in the general formula, wherein Xaa represents an amino acid residue, preferably selected from any (native) amino acid residue residue except serine and Threonine
  VARIANT (18)..(18): Xaa is Xna as defined in the general formula, wherein Xaa represents an amino acid residue, preferably selected from any (native) amino acid residue residue except serine and threonine
  REPEAT (19)..(19): Xaa is Xnb as defined in the general formula, wherein n is 0-5, 5-10, 10-15, 15-20, 20-30 or more for Xnb
  VARIANT (19)..(19): Xaa is Xnb as defined in the general formula, wherein Xaa represents an amino acid residue, preferably selected from any (native) amino acid residue;
XDQXXXXXXXLXLTTPRXX

SEQ ID NO: 5 - protein, 10 aa, Artificial
  Description of sequence  Peptide L-TAT (see Table 1)
GRKKRRQRRR

SEQ ID NO: 6 - protein, 10 aa, Artificial
  Description of sequence Peptide D-TAT (see Table 1)
  MUTAGEN (1)..(10): all amino acids are D-amino acids
RRRQRRKKRG

SEQ ID NO: 7 - protein, 11 aa, Artificial
  Description of sequence Peptide L-generic-TAT (s) (see Table 1)
  misc_feature: General formula NH2-Xnb-RKKRRQRRR-Xnb-COOH (see Table 1)
  VARIANT (1)..(1): Xaa is Xnb as defined in the general formula, wherein Xaa represents an amino acid residue, preferably selected from any (native) amino acid residue;
  REPEAT (1)..(1): Xaa is Xnb as defined in the general formula, wherein n is 0-5, 5-10, 10-15, 15-20, 20-30 or more for Xnb
  VARIANT (11)..(11): Xaa is Xnb as defined in the general formula, wherein Xaa represents an amino acid residue, preferably selected from any (native) amino acid residue;
  REPEAT (11)..(11): Xaa is Xnb as defined in the general formula, wherein n is 0-5, 5-10, 10-15, 15-20, 20-30 or more for Xnb
XRKKRRQRRRX

SEQ ID NO: 8 - protein, 11 aa, Artificial
  Description of sequence Peptide D-generic-TAT (s) (see Table 1)
  misc_feature: General formula NH2-Xnb-RRRQRRKKR-Xnb-COOH
  MUTAGEN (1)..(10): all amino acids are D-amino acids
  VARIANT (1)..(1): Xaa is Xnb as defined in the general formula, wherein Xaa represents an amino acid residue, preferably selected from any (native) amino acid residue;
  REPEAT (1)..(1): Xaa is Xnb as defined in the general formula, wherein n is 0-5, 5-10, 10-15, 15-20, 20-30 or more for Xnb
  VARIANT (11)..(11): Xaa is Xnb as defined in the general formula, wherein Xaa represents an amino acid residue, preferably selected from any (native) amino acid residue;
  REPEAT (11)..(11): Xaa is Xnb as defined in the general formula, wherein n is 0-5, 5-10, 10-15, 15-20, 20-30 or more for Xnb
XRRRQRRKKRX

SEQ ID NO: 9 - protein, 31 aa, Artificial
  Description of sequence L-TAT-IB1 (s) (see Table 1)
GRKKRRQRRRPPRPKRPTTLNLFPQVPRSQD

SEQ ID NO: 10 - protein, 29 aa, Artificial
  Description of sequence Peptide L-TAT (generic) (s) (see Table 1)
  misc_feature: Description of sequence General formula NH2-Xnb-RKKRRQRRR-Xnb-Xna-RPTTLXLXXXXXXXQD-Xnb-COOH
  VARIANT (1)..(1): Xaa is Xnb as defined in the general formula, wherein Xaa represents an amino acid residue, preferably selected from any (native) amino acid residue;
  REPEAT (1)..(1): Xaa is Xnb as defined in the general formula, wherein n is 0-5, 5-10, 10-15, 15-20, 20-30 or more for Xnb
  VARIANT (11)..(11): Xaa is Xnb as defined in the general formula, wherein Xaa represents an amino acid residue, preferably selected from any (native) amino acid residue;
  REPEAT (11)..(11): Xaa is Xnb as defined in the general formula, wherein n is 0-5, 5-10, 10-15, 15-20, 20-30 or more for Xnb
  VARIANT (12)..(12): Xaa is Xna as defined in the general formula, wherein Xaa represents an amino acid residue, preferably selected from any (native) amino acid residue residue except serine and Threonine
  REPEAT (12)..(12): Xaa is Xna as defined in the general formula, wherein n is 0 or 1
  VARIANT (18)..(18): Xaa represents an amino acid residue, preferably selected from any (native) amino acid residue;
  VARIANT (20)..(26): Xaa represents an amino acid residue, preferably selected from any (native) amino acid residue;
  VARIANT (29)..(29): Xaa is Xnb as defined in the general formula, wherein Xaa represents an amino acid residue, preferably selected from any (native) amino acid residue;
  REPEAT (29)..(29): Xaa is Xnb as defined in the general formula, wherein n is 0-5, 5-10, 10-15, 15-20, 20-30 or more for Xnb
XRKKRRQRRRXXRPTTLXLXXXXXXXQDX

SEQ ID NO: 11 - protein, 31 aa, Artificial
  Description of sequence Peptid D-TAT-IB1 (s) (see Table 1)
  MUTAGEN (1)..(31): all amino acids are D-amino acids
DQSRPVQPFLNLTTPRKPRPPRRRQRRKKRG

SEQ ID NO: 12 - protein, 29 aa, Artificial
  Description of sequence Peptid D-TAT (generic) (s) (see Table 1)
  misc_feature: General formula NH2-Xnb-DQXXXXXXXLXLTTPR-Xna-Xnb-RRRQRRKKR-Xnb-COOH,
  MUTAGEN (1)..(19): all amino acids are D-amino acids
  VARIANT (1)..(1): Xaa is Xnb as defined in the general formula, wherein Xaa represents an amino acid residue, preferably selected from any (native) amino acid residue;
  REPEAT (1)..(1): Xaa is Xnb as defined in the general formula, wherein n is 0-5, 5-10, 10-15, 15-20, 20-30 or more for Xnb
  VARIANT (4)..(10): Xaa represents an amino acid residue, preferably selected from any (native) amino acid residue;
  VARIANT (12)..(12): Xaa represents an amino acid residue, preferably selected from any (native) amino acid residue;
  VARIANT (18)..(18): Xaa is Xna as defined in the general formula, wherein Xaa represents an amino acid residue, preferably selected from any (native) amino acid residue residue except serine and threonine
  REPEAT (18)..(18): Xaa is Xna as defined in the general formula, wherein n is 0 or 1
  VARIANT (19)..(19): Xaa is Xnb as defined in the general formula, wherein Xaa represents an amino acid residue, preferably selected from any (native) amino acid residue;
  REPEAT (19)..(19): Xaa is Xnb as defined in the general formula, wherein n is 0-5, 5-10, 10-15, 15-20, 20-30 or more for Xnb
  VARIANT (29)..(29): Xaa is Xnb as defined in the general formula, wherein Xaa represents an amino acid residue, preferably selected from any (native) amino acid residue;
  REPEAT (29)..(29): Xaa is Xnb as defined in the general formula, wherein n is 0-5, 5-10, 10-15, 15-20, 20-30 or more for Xnb
XDQXXXXXXXLXLTTPRXXRRRQRRKKRX

SEQ ID NO: 13 - protein, 29 aa, Artificial
  Description of sequence peptide IB1-long (see Table 1)
PGTGCGDTYRPKRPTTLNLFPQVPRSQDT

SEQ ID NO: 14 - protein, 27 aa, Artificial
  Description of sequence Peptide IB2-long (see Table 1)
IPSPSVEEPHKHRPTTLRLTTLGAQDS

SEQ ID NO: 15 - protein, 29 aa, Artificial
  Description of sequence Peptide derived from c-Jun (see Table 1)
GAYGYSNPKILKQSMTLNLADPVGNLKPH

SEQ ID NO: 16 - protein, 29 aa, Artificial
  Description of sequence Peptide derived from ATF2 (see Table 1)
TNEDHLAVHKHKHEMTLKFGPARNDSVIV

SEQ ID NO: 17 - protein, 23 aa, Artificial
  Description of sequence Peptide L-IB1 (see Table 1)
DTYRPKRPTTLNLFPQVPRSQDT

SEQ ID NO: 18 - protein, 23 aa, Artificial
  Description of sequence Peptide D-IB1 (see Table 1)
  MUTAGEN (1)..(23): all amino acids are D-amino acids
TDQSRPVQPFLNLTTPRKPRYTD

SEQ ID NO: 19 - protein, 19 aa, Artificial
  Description of sequence Peptide L-IB (generic) (see Table 1)
  VARIANT (1)..(1): Xaa is selected from any amino acid residue,
  VARIANT (7)..(7): Xaa is selected from any amino acid residue,
  VARIANT (9)..(15): Xaa is selected from any amino acid residue,
  VARIANT (18)..(18): Xaa is selected from serine or threonine,
  VARIANT (19)..(19): Xaa is selected from any amino acid residue,
XRPTTLXLXXXXXXXQDXX

SEQ ID NO: 20 - protein, 19 aa, Artificial
  Description of sequence Peptide D-IB (generic) (see Table 1)
  MUTAGEN (1)..(19): all amino acids are D-amino acids
  VARIANT (1)..(1): Xaa is selected from any amino acid residue
  VARIANT (2)..(2): Xaa is selected from serine or threonine
  VARIANT (5)..(11): Xaa is selected from any amino acid residue
  VARIANT (13)..(13): Xaa is selected from any amino acid residue
  VARIANT (19)..(19): Xaa is selected from any amino acid residue
XXDQXXXXXXXLXLTTPRX

SEQ ID NO: 21 - protein, 17 aa, Artificial
  Description of sequence Peptide L-generic-TAT (see Table 1)
  VARIANT (1)..(17): Xaa is selected from any amino acid residue
XXXXRKKRRQRRRXXXX

SEQ ID NO: 22 - protein, 17 aa, Artificial
  Description of sequence Peptide D-generic-TAT (see Table 1)
  MUTAGEN (1)..(17): all amino acids are D-amino acids
  VARIANT (1)..(17): Xaa is selected from any amino acid residue
XXXXRRRQRRKKRXXXX

SEQ ID NO: 23 - protein, 35 aa, Artificial
  Description of sequence Peptide L-TAT-IB1 (see Table 1)
GRKKRRQRRRPPDTYRPKRPTTLNLFPQVPRSQDT

SEQ ID NO: 24 - protein, 42 aa, Artificial
  Description of sequence Peptide L-TAT IB (generic) (see Table 1)
  VARIANT (1)..(40): Xaa is selected from any amino acid residue
  VARIANT (41)..(41): Xaa is selected from serine or threonine
  VARIANT (42)..(42): Xaa is selected from any amino acid residue
XXXXXXXRKKRRQRRRXXXXXXXXRPTTLXLXXXXXXXQDXX

SEQ ID NO: 25 - protein, 35 aa, Artificial
  Description of sequence Peptide D-TAT-IB1 (see Table 1)
  MUTAGEN (1)..(35): all amino acids are D-amino acids
TDQSRPVQPFLNLTTPRKPRYTDPPRRRQRRKKRG

SEQ ID NO: 26 - protein, 42 aa, Artificial
  Description of sequence Peptide D-TAT IB (generic) (see Table 1)
  MUTAGEN (1)..(42): all amino acids are D-amino acids
  VARIANT (1)..(1): Xaa is selected from any amino acid residue
  VARIANT (2)..(2): Xaa is selected from serine or threonine
  VARIANT (3)..(42): Xaa is selected from any amino acid residue
XXDQXXXXXXXLXLTTPRXXXXXXXXRRRQRRKKRXXXXXXX

SEQ ID NO: 27 - protein, 30 aa, Artificial
  Description of sequence chimeric peptide sequence L-TAT-IB1(s1) (see Table 1)
RKKRRQRRRPPRPKRPTTLNLFPQVPRSQD

SEQ ID NO: 28 - protein, 30 aa, Artificial
  Description of sequence chimeric peptide sequence L-TAT-IB1(s2) (see Table 1)
  VARIANT (11)..(11): Xaa is selected from glycine or proline
  REPEAT (11)..(11): Xaa is Xnc as defined in the general formula, wherein n is 0-5, 5-10, 10-15, 15-20, 20-30 or more for Xnc
GRKKRRQRRRXRPKRPTTLNLFPQVPRSQD

SEQ ID NO: 29 - protein, 29 aa, Artificial
  Description of sequence chimeric peptide sequence L-TAT-IB1(s3) (see Table 1)
  VARIANT (10)..(10): Xaa is selected from glycine or proline
  REPEAT (10)..(10): Xaa is Xnc as defined in the general formula, wherein n is 0-5, 5-10, 10-15, 15-20, 20-30 or more for Xnc
RKKRRQRRRXRPKRPTTLNLFPQVPRSQD

SEQ ID NO: 30 - protein, 30 aa, Artificial
  Description of sequence chimeric peptide sequence D-TAT-IB1(s1) (see Table 1)
  MUTAGEN (1)..(30): all amino acids are D-amino acids
DQSRPVQPFLNLTTPRKPRPPRRRQRRKKR

SEQ ID NO: 31 - protein, 30 aa, Artificial
  Description of sequence chimeric peptide sequence D-TAT-IB1(s2) (see Table 1)
  MUTAGEN (1)..(30): all amino acids are D-amino acids
  VARIANT (20)..(20): Xaa is selected from glycine or proline
  REPEAT (20)..(20): Xaa is Xnc as defined in the general formula, wherein n is 0-5, 5-10, 10-15, 15-20, 20-30 or more for Xnc
DQSRPVQPFLNLTTPRKPRXRRRQRRKKRG

SEQ ID NO: 32 - protein, 29 aa, Artificial
  Description of sequence chimeric peptide sequence D-TAT-IB1(s3) (see Table 1)
  MUTAGEN (1)..(29): all amino acids are D-amino acids
  VARIANT (20)..(20): Xaa is selected from glycine or proline
  REPEAT (20)..(20): Xaa is Xnc as defined in the general formula, wherein n is 0-5, 5-10, 10-15, 15-20, 20-30 or more for Xnc
DQSRPVQPFLNLTTPRKPRXRRRQRRKKR

SEQ ID NO: 33 - protein, 13 aa, Artificial
  Description of sequence L-IB1(s1) (see Table 1)
TLNLFPQVPRSQD

SEQ ID NO: 34 - protein, 13 aa, Artificial
  Description of sequence L-IB1(s2) (see Table 1)
TTLNLFPQVPRSQ

SEQ ID NO: 35 - protein, 13 aa, Artificial
  Description of sequence L-IB1(s3) (see Table 1)
PTTLNLFPQVPRS

SEQ ID NO: 36 - protein, 13 aa, Artificial
  Description of sequence L-IB1(s4) (see Table 1)
RPTTLNLFPQVPR

SEQ ID NO: 37 - protein, 13 aa, Artificial
  Description of sequence L-IB1(s5) (see Table 1)
KRPTTLNLFPQVP

SEQ ID NO: 38 - protein, 13 aa, Artificial
  Description of sequence L-IB1(s6) (see Table 1)
PKRPTTLNLFPQV

SEQ ID NO: 39 - protein, 13 aa, Artificial
  Description of sequence L-IB1(s7) (see Table 1)
RPKRPTTLNLFPQ

SEQ ID NO: 40 - protein, 12 aa, Artificial
  Description of sequence L-IB1(s8) (see Table 1)
LNLFPQVPRSQD

SEQ ID NO: 41 - protein, 12 aa, Artificial
  Description of sequence L-IB1(s9) (see Table 1)
TLNLFPQVPRSQ

SEQ ID NO: 42 - protein, 12 aa, Artificial
  Description of sequence L-IB1(s10) (see Table 1)
TTLNLFPQVPRS

SEQ ID NO: 43 - protein, 12 aa, Artificial
  Description of sequence L-IB1(s11) (see Table 1)
PTTLNLFPQVPR

SEQ ID NO: 44 - protein, 12 aa, Artificial
  Description of sequence L-IB1(s12) (see Table 1)
RPTTLNLFPQVP

SEQ ID NO: 45 - protein, 12 aa, Artificial
  Description of sequence L-IB1(s13) (see Table 1)
KRPTTLNLFPQV

SEQ ID NO: 46 - protein, 12 aa, Artificial
  Description of sequence L-IB1(s14) (see Table 1)
PKRPTTLNLFPQ

SEQ ID NO: 47 - protein, 12 aa, Artificial
  Description of sequence L-IB1(s15) (see Table 1)
RPKRPTTLNLFP

SEQ ID NO: 48 - protein, 11 aa, Artificial
  Description of sequence L-IB1(s16) (see Table 1)
NLFPQVPRSQD

SEQ ID NO: 49 - protein, 11 aa, Artificial
  Description of sequence L-IB1(s17) (see Table 1)
LNLFPQVPRSQ

SEQ ID NO: 50 - protein, 11 aa, Artificial
  Description of sequence L-IB1(s18) (see Table 1)
TLNLFPQVPRS

SEQ ID NO: 51 - protein, 11 aa, Artificial
  Description of sequence L-IB1(s19) (see Table 1)
TTLNLFPQVPR

SEQ ID NO: 52 - protein, 11 aa, Artificial
  Description of sequence L-IB1(s20) (see Table 1)
PTTLNLFPQVP

SEQ ID NO: 53 - protein, 11 aa, Artificial
  Description of sequence L-IB1(s21) (see Table 1)
RPTTLNLFPQV

SEQ ID NO: 54 - protein, 11 aa, Artificial
  Description of sequence L-IB1(s22) (see Table 1)
KRPTTLNLFPQ

SEQ ID NO: 55 - protein, 11 aa, Artificial
  Description of sequence L-IB1(s23) (see Table 1)
PKRPTTLNLFP

SEQ ID NO: 56 - protein, 11 aa, Artificial
  Description of sequence L-IB1(s24) (see Table 1)
RPKRPTTLNLF

SEQ ID NO: 57 - protein, 10 aa, Artificial
  Description of sequence L-IB1(s25) (see Table 1)
LFPQVPRSQD

SEQ ID NO: 58 - protein, 10 aa, Artificial
  Description of sequence L-IB1(s26) (see Table 1)
NLFPQVPRSQ

SEQ ID NO: 59 - protein, 10 aa, Artificial
  Description of sequence L-IB1(s27) (see Table 1)
LNLFPQVPRS

SEQ ID NO: 60 - protein, 10 aa, Artificial
  Description of sequence L-IB1(s28) (see Table 1)
TLNLFPQVPR

SEQ ID NO: 61 - protein, 10 aa, Artificial
  Description of sequence L-IB1(s29) (see Table 1)
TTLNLFPQVP

SEQ ID NO: 62 - protein, 10 aa, Artificial
  Description of sequence L-IB1(s30) (see Table 1)
PTTLNLFPQV

SEQ ID NO: 63 - protein, 10 aa, Artificial
  Description of sequence L-IB1(s31) (see Table 1)
RPTTLNLFPQ

SEQ ID NO: 64 - protein, 10 aa, Artificial
  Description of sequence L-IB1(s32) (see Table 1)
KRPTTLNLFP

SEQ ID NO: 65 - protein, 10 aa, Artificial
  Description of sequence L-IB1(s33) (see Table 1)
PKRPTTLNLF

SEQ ID NO: 66 - protein, 10 aa, Artificial
  Description of sequence L-IB1(s34) (see Table 1)
RPKRPTTLNL

SEQ ID NO: 67 - protein, 13 aa, Artificial
  Description of sequence D-IB1(s1) (see Table 1)
  MUTAGEN (1)..(13): all amino acids are D-amino acids
QPFLNLTTPRKPR

SEQ ID NO: 68 - protein, 13 aa, Artificial
  Description of sequence D-IB1(s2) (see Table 1)
  MUTAGEN (1)..(13): all amino acids are D-amino acids
VQPFLNLTTPRKP

SEQ ID NO: 69 - protein, 13 aa, Artificial
  Description of sequence D-IB1(s3) (see Table 1)
  MUTAGEN (1)..(13): all amino acids are D-amino acids
PVQPFLNLTTPRK

SEQ ID NO: 70 - protein, 13 aa, Artificial
  Description of sequence D-IB1(s4) (see Table 1)
  MUTAGEN (1)..(13): all amino acids are D-amino acids
RPVQPFLNLTTPR

SEQ ID NO: 71 - protein, 13 aa, Artificial
  Description of sequence D-IB1(s5) (see Table 1)
  MUTAGEN (1)..(13): all amino acids are D-amino acids
SRPVQPFLNLTTP

SEQ ID NO: 72 - protein, 13 aa, Artificial
  Description of sequence D-IB1(s6) (see Table 1)
  MUTAGEN (1)..(13): all amino acids are D-amino acids
QSRPVQPFLNLTT

SEQ ID NO: 73 - protein, 13 aa, Artificial
  Description of sequence D-IB1(s7) (see Table 1)
  MUTAGEN (1)..(13): all amino acids are D-amino acids
DQSRPVQPFLNLT

SEQ ID NO: 74 - protein, 12 aa, Artificial
  Description of sequence D-IB1(s8) (see Table 1)
  MUTAGEN (1)..(12): all amino acids are D-amino acids
PFLNLTTPRKPR

SEQ ID NO: 75 - protein, 12 aa, Artificial
  Description of sequence D-IB1(s9) (see Table 1)
  MUTAGEN (1)..(12): all amino acids are D-amino acids
QPFLNLTTPRKP

SEQ ID NO: 76 - protein, 12 aa, Artificial
  Description of sequence D-IB1(s10) (see Table 1)
  MUTAGEN (1)..(12): all amino acids are D-amino acids
VQPFLNLTTPRK

SEQ ID NO: 77 - protein, 12 aa, Artificial
  Description of sequence D-IB1(s11) (see Table 1)
  MUTAGEN (1)..(12): all amino acids are D-amino acids
PVQPFLNLTTPR

SEQ ID NO: 78 - protein, 12 aa, Artificial
  Description of sequence D-IB1(s12) (see Table 1)
  MUTAGEN (1)..(12): all amino acids are D-amino acids
RPVQPFLNLTTP

SEQ ID NO: 79 - protein, 12 aa, Artificial
  Description of sequence D-IB1(s13) (see Table 1)
  MUTAGEN (1)..(12): all amino acids are D-amino acids
SRPVQPFLNLTT

SEQ ID NO: 80 - protein, 12 aa, Artificial
  Description of sequence D-IB1(s14) (see Table 1)
  MUTAGEN (1)..(12): all amino acids are D-amino acids
QSRPVQPFLNLT

SEQ ID NO: 81 - protein, 12 aa, Artificial
  Description of sequence D-IB1(s15) (see Table 1)
  MUTAGEN (1)..(12): all amino acids are D-amino acids
DQSRPVQPFLNL

SEQ ID NO: 82 - protein, 11 aa, Artificial
  Description of sequence D-IB1(s16) (see Table 1)
  MUTAGEN (1)..(11): all amino acids are D-amino acids
FLNLTTPRKPR

SEQ ID NO: 83 - protein, 11 aa, Artificial
  Description of sequence D-IB1(s17) (see Table 1)
  MUTAGEN (1)..(11): all amino acids are D-amino acids
PFLNLTTPRKP

SEQ ID NO: 84 - protein, 11 aa, Artificial
  Description of sequence D-IB1(s18) (see Table 1)
  MUTAGEN (1)..(11): all amino acids are D-amino acids
QPFLNLTTPRK

SEQ ID NO: 85 - protein, 11 aa, Artificial
  Description of sequence D-IB1(s19) (see Table 1)
  MUTAGEN (1)..(11): all amino acids are D-amino acids
VQPFLNLTTPR

SEQ ID NO: 86 - protein, 11 aa, Artificial
  Description of sequence D-IB1(s20) (see Table 1)
  MUTAGEN (1)..(11): all amino acids are D-amino acids
PVQPFLNLTTP

SEQ ID NO: 87 - protein, 11 aa, Artificial
  Description of sequence D-IB1(s21) (see Table 1)
  MUTAGEN (1)..(11): all amino acids are D-amino acids
RPVQPFLNLTT

SEQ ID NO: 88 - protein, 11 aa, Artificial
  Description of sequence D-IB1(s22) (see Table 1)
  MUTAGEN (1)..(11): all amino acids are D-amino acids
SRPVQPFLNLT

SEQ ID NO: 89 - protein, 11 aa, Artificial
  Description of sequence D-IB1(s23) (see Table 1)
  MUTAGEN (1)..(11): all amino acids are D-amino acids
QSRPVQPFLNL

SEQ ID NO: 90 - protein, 11 aa, Artificial
  Description of sequence D-IB1(s24) (see Table 1)
  MUTAGEN (1)..(11): all amino acids are D-amino acids
DQSRPVQPFLN

SEQ ID NO: 91 - protein, 10 aa, Artificial
  Description of sequence D-IB1(s25) (see Table 1)
  MUTAGEN (1)..(10): all amino acids are D-amino acids
DQSRPVQPFL

SEQ ID NO: 92 - protein, 10 aa, Artificial
  Description of sequence D-IB1(s26) (see Table 1)
  MUTAGEN (1)..(10): all amino acids are D-amino acids
QSRPVQPFLN

SEQ ID NO: 93 - protein, 10 aa, Artificial
  Description of sequence D-IB1(s27) (see Table 1)
  MUTAGEN (1)..(10): all amino acids are D-amino acids
SRPVQPFLNL

SEQ ID NO: 94 - protein, 10 aa, Artificial
  Description of sequence D-IB1(s28) (see Table 1)
  MUTAGEN (1)..(10): all amino acids are D-amino acids
RPVQPFLNLT

SEQ ID NO: 95 - protein, 10 aa, Artificial
  Description of sequence D-IB1(s29) (see Table 1)
  MUTAGEN (1)..(10): all amino acids are D-amino acids
PVQPFLNLTT

SEQ ID NO: 96 - protein, 10 aa, Artificial
  Description of sequence D-IB1(s30) (see Table 1)
  MUTAGEN (1)..(10): all amino acids are D-amino acids
VQPFLNLTTP

SEQ ID NO: 97 - protein, 10 aa, Artificial
  Description of sequence D-IB1(s31) (see Table 1)
  MUTAGEN (1)..(10): all amino acids are D-amino acids
QPFLNLTTPR

SEQ ID NO: 98 - protein, 10 aa, Artificial
  Description of sequence D-IB1(s32) (see Table 1)
  MUTAGEN (1)..(10): all amino acids are D-amino acids
PFLNLTTPRK

SEQ ID NO: 99 - protein, 10 aa, Artificial
  Description of sequence D-IB1(s33) (see Table 1)
  MUTAGEN (1)..(10): all amino acids are D-amino acids
FLNLTTPRKP

SEQ ID NO: 100 - protein, 10 aa, Artificial
  Description of sequence D-IB1(s34) (see Table 1)
  MUTAGEN (1)..(10): all amino acids are D-amino acids
LNLTTPRKPR

SEQ ID NO: 101 - DNA, 21 bp, Artificial
  Description of sequence ap-1 doubled labeled probe (see p. 66)
cgcttgatgagtcagccggaa

SEQ ID NO: 102 - DNA, 2953 bp, Artificial
  description of sequence rat IB1 cDNA sequence and its predicted amino acid sequence (see Figure 1)
  CDS (108)..(2252)
ccgccccagctcagtccgaaccccgcggcggcggcggcctcctccacacgcctccacctccgccgccgccgccgccgccgccgcctcccgcgccgctctccgcccggatggccaggctgagcccgggaatggcggagcgagagagcggcctgagcgggggtgccgcgtccccaccggccgcttccccattcctgggactgcacatcgcgtcgcctcccaatttcaggctcacccatgatatcagcctggaggagtttgaggatgaagacctttcggagatcactgatgagtgtggcatcagcctgcagtgcaaagacaccttgtctctccggcccccgcgcgccgggctactgtctgcgggtagcagcggtagcgcggggagccggctgcaggcggagatgctgcagatggacctgatcgacgcggcaagtgacactccgggcgccgaggacgacgaagaggacgacgacgagctcgctgcccaacggccaggagtggggccttccaaagccgagtctggccaggagccggcgtctcgcagccagggtcagggccagggccccggcacaggctgcggagacacctaccggcccaagaggcctaccacgctcaaccttttcccgcaggtgccgcggtctcaggacacgctgaataataactctttaggcaaaaagcacagttggcaggaccgtgtgtctcgatcatcctcccctctgaagacaggggagcagacgcctccacatgaacatatctgcctgagtgatgagctgccgccccagggcagtcctgttcccacccaggatcgtggcacttccaccgacagcccttgtcgccgtactgcagccacccagatggcacctccaagtggtccccctgccactgcacctggtggccggggccactcccatcgagatcggtccatatcagcagatgtgcggctcgaggcgactgaggagatctacctgaccccagtgcagaggcccccagaccctgcagaacccacctccaccttcttgccacccactgagagccggatgtctgtcagctcggatcctgaccctgccgcttactctgtaactgcagggcgaccgcacccttccatcagtgaagaggatgagggcttcgactgtctgtcatccccagagcaagctgagccaccaggtggagggtggcggggaagcctcggggagccaccaccgcctccacgggcctcactgagctcggacaccagcgcactgtcctacgactctgtcaagtacacactggtggtggatgagcatgcccagcttgagttggtgagcctgcggccatgttttggagattacagtgacgaaagcgactctgccactgtctatgacaactgtgcctctgcctcctcgccctacgagtcagccattggtgaggaatatgaggaggcccctcaaccccggcctcccacctgcctgtcagaggactccacaccggatgagcctgacgtccacttctctaagaagtttctgaatgtcttcatgagtggccgctctcgttcctccagtgccgagtcctttgggctgttctcctgtgtcatcaatggggaggagcatgagcaaacccatcgggctatattcaggtttgtgcctcggcatgaagatgaacttgagctggaagtggacgaccctctgctggtggagctgcaggcagaagactattggtatgaggcctataacatgcgcactggagcccgtggtgtctttcctgcctactatgccattgaggtcaccaaggagcctgagcacatggcagcccttgccaaaaacagcgactggattgaccagttccgggtgaagttcctgggctctgtccaggttccttatcacaagggcaatgatgtcctctgtgctgctatgcaaaagatcgccaccacccgccggctcaccgtgcactttaacccgccctccagctgtgtccttgaaatcagcgttaggggtgtcaagataggtgtcaaagctgatgaagctcaggaggccaagggaaataaatgtagccactttttccagctaaaaaacatctctttctgtgggtaccatccaaagaacaacaagtactttgggtttatcactaagcaccctgctgaccaccggtttgcctgccatgtctttgtgtctgaagattccaccaaagccctggcagagtctgtggggcgtgcatttcagcagttctacaagcaatttgtggaatatacctgtcctacagaagatatctacttggagtagcagcaacccccctctctgcagcccctcagccccaggccagtactaggacagctgactgctgacaggatgttgtactgccacgagagaatgggggagtgagggctgttggggtcggggggcaggggtttggggagaggcagatgcagtttattgtaatatatggggttagattaatctatggaggacagtacaggctctctcggggctggggaagggcagggctggggtgggggtcaggcatctggccacaaaggggtcccctagggacagaggcgctgcaccatcctgggcttgtttcatactagaggccctggctttctggctcttgggtcctgccttgacaaagcccagccacctggaagtgtcaccttcccttgtccacctcacccagtgccctgagctcatgctgagcccaagcacctccgaaggactttccagtaaggaaatggcaacatgtgacagtgagaccctgttctcatctgtggggctccggcagctccgacccccagcctggccagcacgctgaccctggcaagcttgtgtgttcaaagaaggagagggccacagcaagccctgcctgccagggaaggttccctctcagctggccccagccaactggtcactgtcttgtcacctggctactactattaaagtgccatttcttgtctgaaaaaaaaaaaaaaaaaaaaaaaaaactcgag

SEQ ID NO: 103 - protein, 714 aa, Artificial
  description of sequence Protein encoded by Exon-Intron Boundary of the rIB1 Gene - Splice donor
MARLSPGMAERESGLSGGAASPPAASPFLGLHIASPPNFRLTHDISLEEFEDEDLSEITDECGISLQCKDTLSLRPPRAGLLSAGSSGSAGSRLQAEMLQMDLIDAASDTPGAEDDEEDDDELAAQRPGVGPSKAESGQEPASRSQGQGQGPGTGCGDTYRPKRPTTLNLFPQVPRSQDTLNNNSLGKKHSWQDRVSRSSSPLKTGEQTPPHEHICLSDELPPQGSPVPTQDRGTSTDSPCRRTAATQMAPPSGPPATAPGGRGHSHRDRSISADVRLEATEEIYLTPVQRPPDPAEPTSTFLPPTESRMSVSSDPDPAAYSVTAGRPHPSISEEDEGFDCLSSPEQAEPPGGGWRGSLGEPPPPPRASLSSDTSALSYDSVKYTLVVDEHAQLELVSLRPCFGDYSDESDSATVYDNCASASSPYESAIGEEYEEAPQPRPPTCLSEDSTPDEPDVHFSKKFLNVFMSGRSRSSSAESFGLFSCVINGEEHEQTHRAIFRFVPRHEDELELEVDDPLLVELQAEDYWYEAYNMRTGARGVFPAYYAIEVTKEPEHMAALAKNSDWIDQFRVKFLGSVQVPYHKGNDVLCAAMQKIATTRRLTVHFNPPSSCVLEISVRGVKIGVKADEAQEAKGNKCSHFFQLKNISFCGYHPKNNKYFGFITKHPADHRFACHVFVSEDSTKALAESVGRAFQQFYKQFVEYTCPTEDIYLE

SEQ ID NO: 104 - protein, 711 aa, Homo sapiens
  description of sequence human IB1 protein sequence
MAERESGGLGGGAASPPAASPFLGLHIASPPNFRLTHDISLEEFEDEDLSEITDECGISLQCKDTLSLRPPRAGLLSAGGGGAGSRLQAEMLQMDLIDATGDTPGAEDDEEDDDEERAARRPGAGPPKAESGQEPASRGQGQSQGQSQGPGSGDTYRPKRPTTLNLFPQVPRSQDTLNNNSLGKKHSWQDRVSRSSSPLKTGEQTPPHEHICLSDELPPQSGPAPTTDRGTSTDSPCRRSTATQMAPPGGPPAAPPGGRGHSHRDRIHYQADVRLEATEEIYLTPVQRPPDAAEPTSAFLPPTESRMSVSSDPDPAAYPSTAGRPHPSISEEEEGFDCLSSPERAEPPGGGWRGSLGEPPPPPRASLSSDTSALSYDSVKYTLVVDEHAQLELVSLRPCFGDYSDESDSATVYDNCASVSSPYESAIGEEYEEAPRPQPPACLSEDSTPDEPDVHFSKKFLNVFMSGRSRSSSAESFGLFSCIINGEEQEQTHRAIFRFVPRHEDELELEVDDPLLVELQAEDYWYEAYNMRTGARGVFPAYYAIEVTKEPEHMAALAKNSDWVDQFRVKFLGSVQVPYHKGNDVLCAAMQKIATTRRLTVHFNPPSSCVLEISVRGVKIGVKADDSQEAKGNKCSHFFQLKNISFCGYHPKNNKYFGFITKHPADHRFACHVFVSEDSTKALAESVGRAFQQFYKQFVEYTCPTEDIYLE

SEQ ID NO: 105 - DNA, 2136 bp, Homo sapiens
  description of sequence nucleic acid sequence encoding human IB1 protein
atggcggagcgagaaagcggcggcctgggagggggggccgcgtccccgcccgccgcctccccgttcctggggctgcacatcgcttcgcctcccaatttcaggctcacccatgacatcagcctggaggagtttgaggatgaagacctctcggagatcactgatgagtgtggcatcagcttacagtgcaaagacaccctgtccttacggcccccgcgcgccgggctgctctctgcgggcggcggcggcgcggggagccggttgcaggccgagatgctgcagatggacctgatcgacgcgacgggggacactcccggggccgaggacgacgaggaggacgacgacgaggagcgcgcggcccggcggccgggagcggggccgcccaaggccgagtccggccaggagccggcgtcccgcggccagggccagagccaaggccagagccagggcccgggcagcggggacacgtaccggcccaagcggcccaccacgctcaacctctttccgcaggtgccgcggtctcaggacacactgaataataattctctgggcaaaaagcacagttggcaggatcgggtgtctcgatcatcctcacccctgaagacaggggagcagacaccaccgcatgaacacatctgcctgagcgatgagctgcccccccagagcggccccgcccccaccacagatcgaggcacctccaccgacagcccttgccgccgcagcacagccacccagatggcacctccgggtggtccccctgctgccccgcctgggggtcggggccactcgcatcgagaccgaatccactaccaggccgatgtgcgactagaggccactgaggagatctacctgaccccagtgcagaggcccccagacgctgcagagcccacctccgccttcctgccgcccactgagagccggatgtcagtcagctccgatccagaccctgccgcctacccctccacggcagggcggccgcacccctccatcagtgaagaggaagagggcttcgactgcctgtcgtccccagagcgggctgagcccccaggcggagggtggcgggggagcctgggggagccgccgccacctccacgggcctctctgagctcggacaccagcgccctgtcctatgactctgtcaagtacacgctggtggtagatgagcatgcacagctggagctggtgagcctgcggccgtgcttcggagactacagtgacgagagtgactctgccaccgtctatgacaactgtgcctccgtctcctcgccctatgagtcggccatcggagaggaatatgaggaggccccgcggccccagccccctgcctgcctctccgaggactccacgcctgatgaacccgacgtccatttctccaagaaattcctgaacgtcttcatgagtggccgctcccgctcctccagtgctgagtccttcgggctgttctcctgcatcatcaacggggaggagcaggagcagacccaccgggccatattcaggtttgtgcctcgacacgaagacgaacttgagctggaagtggatgaccctctgctagtggagctccaggctgaagactactggtacgaggcctacaacatgcgcactggtgcccggggtgtctttcctgcctattacgccatcgaggtcaccaaggagcccgagcacatggcagccctggccaaaaacagtgactgggtggaccagttccgggtgaagttcctgggctcagtccaggttccctatcacaagggcaatgacgtcctctgtgctgctatgcaaaagattgccaccacccgccggctcaccgtgcactttaacccgccctccagctgtgtcctggagatcagcgtgcggggtgtgaagataggcgtcaaggccgatgactcccaggaggccaaggggaataaatgtagccactttttccagttaaaaaacatctctttctgcggatatcatccaaagaacaacaagtactttgggttcatcaccaagcaccccgccgaccaccggtttgcctgccacgtctttgtgtctgaagactccaccaaagccctggcagagtccgtggggagagcattccagcagttctacaagcagtttgtggagtacacctgccccacagaagatatctacctggagtag